SEQUENCE LISTING (1 sequence)

SEQ ID NO: 1 - DNA, 34 bp, Unknown Organism
  Description of Unknown Organism Unknown LoxP sequence
ataacttcgtatagcatacattatacgaagttat

(19) United States
(12) Patent Application Publication
(10) Pub. No.: US 2005/0260748 A1
(43) Pub. Date: Nov. 24, 2005

(54) Adult stem cells and uses thereof

(75) Inventors: Chia-Cheng Chang, Okemos, MI (US); Jin-Lian Tsai, Kaohsiung City (TW); Kung-Kai Kuo, Kaohsiung City (TW); Tsai-Ming Lin, Kaohsiung City (TW)

Correspondence Address: WILMER CUTLER PICKERING HALE AND DORR LLP, 60 STATE STREET, BOSTON, MA, 02109, US

(73) Assignee: Michigan State University

(21) Appl. No.: 11/069,328

(22) Filed: Feb. 28, 2005

Related U.S. Application Data
(60) Provisional application No. 60/577,815, filed on Jun. 8, 2004.
(60) Provisional application No. 60/548,340, filed on Feb. 27, 2004.

(51) Int. Cl. C12N 5/08

(52) U.S. Cl. 435/366

(57) ABSTRACT

Disclosed are compositions and methods for isolating, immortalizing and differentiating adult stem cells, for example, particular human clonal adult liver stem cells or adipose stem cells, including specialized cell culture media for the isolation and propagation of such stem cells. Also disclosed are methods of screening for toxicity, carcinogenicity and therapeutic activity using such stem cells and immortalized or differentiated derivatives thereof. In addition, methods of treatment using such stem cells and their differentiated or immortalized derivatives thereof are disclosed.

### CROSS REFERENCE TO RELATED APPLICATIONS

This Application claims the benefit of priority to U.S. Provisional Application No. 60/577,815, filed Jun. 8, 2004, and to U.S. Provisional Application No. 60/548,340, filed Feb. 27, 2004.

### 1. FIELD OF THE INVENTION

This invention relates to the field of medicine. In particular, this invention relates to methods and compositions for isolating and culturing adult stem cells and immortalized derivatives thereof. The invention further relates to methods and compositions for treating diseases and disorders using adult stem cells or derivatives thereof.

### 2. BACKGROUND OF THE INVENTION

Stem cells are relatively unspecialized cells that renew themselves through cell division. Under certain physiologic or experimental conditions, a stem cell can be induced to become a more differentiated cell exhibiting the properties of a specific cell type. These features make stem cells useful for cell-based therapies to treat disease. Human mesenchymal stem cells (MSCs) have been isolated from bone marrow and are useful, e.g., for tissue engineering. MSCs have been shown to differentiate into adipocytes, chondrocytes, myoblasts, and osteoblasts, and may be useful for tissue engineering.

An example of a tissue amenable to stem cell-based therapy is the liver. The liver is a vital organ essential for the metabolism of proteins, carbohydrates and fat. The liver also functions as a gland by secreting digestive bile, and a site of synthesis of urea and ketone bodies as well as critical blood proteins such as fibrinogen, prothrombin and serum albumin. The liver plays a central and critical role in detoxification of the blood. The major cells of the liver carrying out these functions include the hepatocytes, as well as Kupffer cells of the reticulo-endothelial system, which are derived from the bile duct cannuliculae, and fibroblasts, which are particularly important in the pathogenesis of liver cirrhosis.

The mammalian adult liver has a remarkable ability to recover after hepatotoxic injury or partial hepatectomy (Michalopoulos et al. (1997) Science 276:60-66; Alison (1998) Curr. Opin. Cell Biol. 10:710-715; Fausto (2000) J. Hepatol. 32:19-31). There is some evidence that such liver regeneration following injury is accomplished by the proliferation of mature hepatocytes (Michalopoulos et al. (1997) Science 276:60-66; Overturf et al. (1997) Am. J. Pathol. 151:1273-1280). However there are limits to the regenerative capability of the human liver in response to damage. Furthermore, mature hepatocytes are difficult to maintain and grow in vitro (Runge et al (2000) Biochem. Biophys. Res. Commun. 269:46-53) (except for the small hepatocytes isolated from adult rats, which are able to grow for several weeks (Tateno et al. (2000) Hepatol. 31:65-74)), so that replacement of liver cell function using ex vivo propagated hepatocytes has found limited application.

There are a multitude of diseases and disorders that necessitate complete replacement of the liver or a supplanting of its functions. Indeed, cirrhosis and other liver diseases take the lives of over 25,000 Americans each year and rank eighth as a cause of death in the United States (see nlm.nih.gov/medlineplus/liverdiseases.html). Furthermore, the hepatitis A, B, and C viruses can cause debilitating damage to the liver, and hepatitis B virus can lead to the development of hepatocellular carcinoma (primary liver cancer), the leading cause of cancer death in the world, and, particularly, in the East where hepatitis B is endemic (see The Liver Disorders Sourcebook by Howard Worman (1999) McGraw-Hill, Columbus, Ohio).

One method of treating liver disease has been to remove the affected organ and replace it with healthy tissue from a donor. However, treatment by liver transplantation is limited by the availability of suitable donor tissue and complications resulting from immune rejection. Accordingly, methods for replacing adult human hepatocyte function, particularly by replacing diseased liver tissue with healthy hepatocytes having the same genetic identity, would be useful for the medical treatment of innumerable debilitating liver conditions.

Adult liver stem cells, or precursor cells, derived from the adult liver would be valuable for cell transplantation, tissue engineering of bioartificial organ and gene therapy in the therapeutic treatment of patient suffering liver failure and/or cirrhosis. While Malhi et al., have reported the isolation of epithelial progenitor/stem cells from fetal human liver (Malhi et al. (2002) J. Cell Sci. 115:2679-2688), these cell cultures were not derived from single cells (i.e., were not clonal) and formed colonies only on irradiated autologous feeder cells but not on tissue culture plastics. Accordingly, methods for the efficient isolation of clonal human adult liver stem cells would be useful in the treatment of a multitude of liver diseases and disorders.

Adipose tissues have been shown to contain mesenchymal precursor cells (stem cells). The isolation and growth of multipotential cells from human adipose tissues, termed processed lipoaspirate (PLA), that could differentiate into adipogenic, chondrogenic, myogenic and osteogenic cells has been reported (Zuk et al. (2001) Tissue Engineering 7:211-228; Zuk et al. (2002) Molecular Biol. of the Cell 13:4279-4295). These studies used the high calcium (1.8 mM) Dulbecco's Modified Eagle Medium (DMEM) supplemented with 10% FBS to grow PLA cells. New methods of obtaining and culturing such multipotential stem cells would be useful, e.g., to obtain a large quantity of cells with high differentiation potential for use in the treatment of a variety of disorders related to the cell types into which adipose cells can differentiate.

### 3. SUMMARY OF THE INVENTION

It has been discovered that particular cell culture media formulations and methods are useful for isolating and propagating stem cells, including adult stem cells, e.g., stem cells derived from tissues including liver and adipose tissue. Based in part on this and related findings disclosed herein, the invention provides novel methods and compositions for obtaining, growing, and propagating adult stem cells. In particular, the invention includes novel methods and compositions for obtaining, growing, and propagating adult cells, e.g., liver stem cells, which allow for the facile isolation, and further manipulation, of such cells from small amounts of adult tissue such as the amounts of tissue obtained in liver biopsy or subcutaneous fat tissue. The invention thereby provides for such adult stem cells themselves, as well as immortalized and/or differentiated (or trans-differentiated) derivatives of such cells. The invention further provides for methods of using such cells in toxological, carcinogen and drug screening methods, as well as in therapeutic applications where an organ function (such as liver function in a subject having a liver disease), cellular function, disorder or dysfunction, is replaced or otherwise supplanted using such cells.

In one aspect, the invention provides a method of obtaining isolated adult liver stem cells from a population of dissociated cells from an adult liver tissue by, first, culturing the population of dissociated liver cells in a cell culture medium that has a low calcium concentration and an effective amount of N-acetyl-L-cysteine, nicotinamide, and/or an antioxidant. The population of cells is then allowed to develop into colonies of adult liver stem cells in this cell culture medium, thereby yielding a population of isolated adult liver stem cells. In certain embodiments of this method, the isolated liver stem cells are primate in origin. In useful embodiments, the primate is human.

In some embodiments of this method, the isolated adult liver stem cells are clonal in origin. In other embodiments, the isolated adult liver stem cells are capable of forming colonies on tissue culture plastic, and may also be obtained without the use of feeder cells. In certain preferred embodiment, the population of isolated adult liver cells has a high proliferation potential, such as a high proliferation potential that is equivalent to about 48 or more cell divisions (e.g., at least 48 cell divisions).

In another embodiment, the method further includes the step of immortalizing the adult liver stem cells obtained by the above method, for example, by transforming them with an immortalizing gene. Immortalizing genes for use in these embodiments include the SV40 large T-antigen, as well as dominant-negative p53, dominant-negative RB, hTERT, adenovirus E1a, adenovirus E1b, papilloma virus E6, or papilloma virus E7.

In an embodiment of the invention, the cell culture medium has a low calcium concentration of less than about 0.3 mM, less than about 0.2 mM, or less than about 0.1 mM. In other embodiments, the low calcium concentration of the medium is about 0.04 mM to about 0.18 mM, about 0.06 mM to about 0.12 mM, or about 0.08 mM to about 0.10 mM. In another embodiment, the low calcium concentration of the cell culture medium is about 0.09 mM.

In another embodiment of the invention, the cell culture medium includes an antioxidant agent, such as vitamin C. The vitamin C may be provided in any form, for example, as L-ascorbic acid-2-phosphate. In one embodiment, the L-ascorbic acid-2-phosphate is provided at a concentration of at least about 0.05 mM. In another, it is provided at about 0.2 mM. In still other embodiments, the antioxidant included in the cell culture medium is vitamin E, N-acetyl-L-cysteine, resveratrol, coenzyme Q, alpha-lipoic acid, lycopene, bioflavonoids, or quercetin.

In still another embodiment, the cell culture medium includes N-acetyl-L- cysteine. In a particular embodiment, the N-acetyl-L-cysteine is supplied at a concentration of at least about 0.5 mM. In other embodiment, the N-acetyl-L-cysteine concentration is about 2 mM.

In still another embodiment, the cell culture medium includes nicotinamide. In a particular embodiment, the nicotinamide is supplied at a concentration of at least about 1 mM to 10 mM. In other embodiment, the nicotinamide concentration is about 5 mM.

In another embodiment, the dissociated liver cells are cultured in a medium containing an effective amount of at least two agents, such as N-acetyl-L-cysteine and nicotinamide, or N-acetyl-L-cysteine and an antioxidant, or nicotinamide and an antioxidant. In other particular embodiments the dissociated liver cells are cultured in a medium comprising an effective amount of all three of the agents N-acetyl-L-cysteine, nicotinamide, and an antioxidant. In certain embodiments, the N-acetyl-L-cysteine is supplied at about 2 mM in the cell culture media. In other embodiments, the nicotinamide is supplied at about 5 mM to 10 mM in the cell culture media. In still other embodiments, the L-ascorbic acid-2-phosphate is supplied at about 0.2 mM in the cell culture media.

In yet other embodiments, the cell culture medium further includes other agents, such as a growth factor or hormone such as EGF, insulin, hydrocortisone, 3,3′,5-triiodo-D.L-thyronine, or fetal bovine serum (to provide those factors and/or hormones contained within fetal bovine serum). In certain embodiments, the cell culture medium includes, for example, 5 ng/ml of recombinant human EGF, 5 μg/ml of insulin, 74 ng/ml of hydrocortisone, 10 nM 3,3′, 5-triiodo-D.L-thyronine, 20-50 μg/ml bovine pituitary extract, or 5-10% fetal bovine serum.

In another embodiment, the invention provides methods for the further differentiation of the isolated adult liver stem cells so that these cells express one or more hepatocyte-specific function. In certain embodiments, the isolated adult liver stem cells are differentiated to form hepatocytes (e.g., mature hepatocytes as judged by morphology and/or gene or activity expression patterns. In one embodiment, the hepatocyte-specific function is gap junctional intercellular communication (GJIC), or P450, glucose-6-phosphatase, catalase, albumin, or P-glycoprotein expression.

In particular embodiments, the adult liver stem cells are differentiated using a cell culture medium having a calcium ion concentration of at least about 0.6 mM calcium. In other embodiments, the adult liver stem cells are differentiated by contacting them with a hepatocyte differentiation agent, such as hepatocyte growth factor, phenobarbitol, or n- butyrate.

In yet another embodiment of this aspect of the invention, the differentiated adult liver stem cells expressing one or more hepatocyte-specific functions are administered to a subject in need of such treatment. In another embodiment, the isolated adult liver stem cells are administered to such a subject in need thereof (e.g., because of a disease, disorder or other dysfunction of the liver). In particular embodiments, the disease, disorder or other dysfunction of the liver to be treated is chronic hepatitis, cirrhosis, liver cancer (adenocarcinoma, metastatic liver cancer or cholangiocarcinoma), Alagille syndrome, alpha 1-antitrypsin deficiency, autoimmune hepatitis, biliary atresia, cystic disease of the liver (choledochal cysts, Caroli's syndrome, congenital hepatic fibrosis, and polycystic liver disease), hemochromatosis, hepatitis A, hepatitis B, hepatitis C, neonatal hepatitis, porphyria, primary biliary cirrhosis, primary sclerosing cholangitis, tyrosinemia, type I glycogen storage disease, or Wilson's disease.

In another aspect, the invention provides a method of obtaining isolated adult liver stem cells by, first, dissociating cells from an adult liver tissue, then culturing the population of cells so obtained in a cell culture medium that includes a low calcium concentration and an effective amount of at least one active agent. The active agent may be one that promotes intracellular glutathione synthesis, or one that inhibits poly ADP- ribose polymerase, or one that is an antioxidant. The adult liver stem cells are then allowed to develop into colonies in this cell culture medium, so that a population of isolated adult liver stem cells results.

In particular embodiments of this aspect of the invention, the population of dissociated liver cells are cultured in a medium that contains an effective amount of at least two of these active agents (e.g., an agent that promotes intracellular glutathione synthesis and an inhibitor of poly ADP-ribose polymerase, or an agent that promotes intracellular glutathione synthesis and an antioxidant, or an inhibitor of poly ADP-ribose polymerase and an antioxidant). In another, useful embodiment, the population of dissociated liver cells is cultured in a medium that contains all three active agents (i.e, an agent that promotes intracellular glutathione synthesis, an inhibitor of poly ADP-ribose polymerase, and an antioxidant). In certain embodiments, the population of dissociated liver cells are cultured in a medium that contains about 2 mM N-acetyl-L-cysteine, about 5 mM nicotinamide, and about 0.2 mM L-ascorbic acid-2-phosphate.

In another aspect, the invention provides cell culture media compositions and formulation having a low calcium ion concentration and an effective amount of one or more of another factor such as N-acetyl-L-cysteine, nicotinamide, or an antioxidant. In certain embodiments, the cell culture medium includes an effective amount of at least two of these factors (i.e., N-acetyl-L-cysteine and nicotinamide, or N-acetyl-L-cysteine and an antioxidant, or nicotinamide and an antioxidant). In certain other embodiments, the cell culture medium includes all three of these components (i.e., N-acetyl-L-cysteine, nicotinamide, and an antioxidant). In other useful embodiments, the cell culture medium includes about 2 mM N-acetyl-L-cysteine, about 5 mM-10 mM nicotinamide, and about 0.2 mM L-ascorbic acid-2-phosphate.

In another embodiment of this aspect of the invention, the cell culture medium has a low calcium ion concentration. In particular embodiments, the calcium ion concentration is less than about 0.2 mM. In certain other embodiments, the low calcium ion concentration is about 0.04 mM to about 0.18 mM, or about 0.08 mM to about 0.10 mM. In certain embodiments, the cell culture media has a low calcium ion concentration of about 0.09 mM.

In another aspect, the invention provides a cell culture medium that includes a low calcium ion concentration and an effective amount of one or more agents having a specific activity. In one embodiment, the cell culture medium includes an agent that promotes intracellular glutathione synthesis, such as N-acetyl-L-cysteine (e.g., 2 mM N- acetyl-L-cysteine). In another embodiment, the cell culture medium includes an agent that inhibits poly ADP-ribose polymerase, such as nicotinamide (e.g., 5 mM nicotinamide). In yet another embodiment, the cell culture medium includes an antioxidant, such as L-ascorbic acid-2-phosphate (e.g., 0.2 mM L-ascorbic acid-2-phosphate). In certain embodiments, the cell culture medium at least two such agents (e.g. an agent that promotes intracellular glutathione synthesis and an inhibitor of poly ADP-ribose polymerase, or an agent that promotes intracellular glutathione synthesis and an antioxidant, or an inhibitor of poly ADP-ribose polymerase and an antioxidant). In still other embodiments, the cell culture medium contains an agent that promotes intracellular glutathione synthesis, an inhibitor of poly ADP-ribose polymerase, and an antioxidant.

In another embodiment of this aspect of the invention, the cell culture medium has a low calcium ion concentration of less than about 0.2 mM. In other embodiments, the low calcium ion concentration is about 0.04 mM to about 0.18 mM, or about 0.08 mM to about 0.10 mM. In embodiments, the low calcium ion concentration is about 0.09 mM.

In another aspect, the invention provides a cell culture medium for adult human liver stem cells that includes a calcium ion concentration of not more than about 0.5 mM, or 0 to about 0.5 mM, as well as at least about 1 mM N-acetyl-L-cysteine, at least about 1 mM nicotinamide, and an effective amount of an antioxidant agent, and, in particular embodiments, this cell culture medium is sufficient for culturing adult human liver stem cells. In particular embodiments of this aspect, the calcium concentration is not more than about 0.2 mM, or 0 to about 0.2 mM. In other embodiments, the calcium concentration is not more than about 0.1 mM, or 0 to about 0.1 mM. In still other embodiments, the calcium ion concentration is about 0.05 mM to about 0.1 mM.

In another embodiment of this aspect of the invention, the antioxidant agent included in the cell culture medium is vitamin C. In one embodiment, the vitamin C is provided as L-ascorbic acid-2-phosphate (e.g., at a concentration of at least about 0.1 mM, such as about 0.2 mM). In certain other useful embodiments, the antioxidant included in the cell culture medium is vitamin E, N-acetyl-L-cysteine, resveratrol, coenzyme Q, alpha-lipoic acid, lycopene, bioflavonoids, or quercetin.

In certain other embodiments, the cell culture medium includes N-acetyl-L- cysteine, and the N-acetyl-L-cysteine is supplied at a concentration of at least about 1 mM.

In yet other embodiments, the cell culture medium includes nicotinamide, and the N-nicotinamide is supplied at a concentration of at least about 2 mM.

In other embodiment, the cell culture medium also contains a growth factor or hormone or mixture of such, such as EGF, insulin, hydrocortisone, 3,3′, 5-triiodo-D, L- thyronine, bovine pituitary extract, or fetal bovine serum. In particular embodiments, the cell culture medium includes 5 ng/ml of recombinant human EGF, 5 μg/ml of insulin, 74 ng/ml of hydrocortisone, 10 nM 3,3′, 5-triiodo-D.L-thyronine, 50 μg/ml bovine pituitary extract, or 10% fetal bovine serum.

In another aspect, the invention provides adult liver stem cells, particularly isolated adult liver stem cells, obtained by, first, culturing the population of dissociated liver cells in a cell culture medium that has a low calcium concentration and an effective amount of N-acetyl-L-cysteine, nicotinamide, and/or an antioxidant. The population of isolated cells is obtained by then allowing the adult liver stem cells to develop into colonies in this cell culture medium. In certain embodiments, the isolated liver stem cells are primate in origin. In some embodiments, the primate is human. In some embodiments, the isolated adult human liver stem cells are clonal in origin.

In other embodiments of this aspect, the adult human liver stem cells of the invention are obtained by the method described above, but are additionally immortalized by transformation with an immortalizing gene. In certain embodiments, the immortalized adult human liver stem cells are immortalized with SV40 large T-antigen.

In certain embodiments of this aspect of the invention, the adult human liver stem cells of the invention are further differentiated so that they express one or more hepatocyte-specific functions. These differentiated adult human liver stem cells may be obtained from immortalized or nonimmortalized adult human liver stem cells. In particular embodiments, the isolated adult liver stem cells are differentiated into hepatocytes. In certain embodiments, the adult human liver stem cells are characterized by their lack of a gap-junction intercellular communication activity. In particular embodiments, the lack of a gap-junction intercellular communication (GJIC) activity is specifically characterized by the absence of expression of a GJIC protein, such as connexin 26 or connexin 43. In other embodiments, the adult human liver stem cells are characterized by the expression of a marker such as Oct-4, alpha-fetoprotein, Thy-1, or vimentin. In particular embodiments, the adult human liver stem cells express two or more of these markers. In certain embodiments, the adult human liver stem cells express Oct-4, alpha-fetoprotein, Thy-1, and vimentin.

In another aspect, the invention provides adult human liver stem cells that are characterized by their lack of a gap-junction intercellular communication activity. In particular embodiments, the lack of a gap-junction intercellular communication (GJIC) activity is specifically characterized by the absence of expression of a GJIC protein, such as connexin 26 or connexin 43. In other embodiments, the adult human liver stem cells are characterized by the expression of an adult stem cell marker such as Oct-4, alpha- fetoprotein, Thy-1, or vimentin. In particular embodiments, the adult human liver stem cells express two or more of these markers. In some embodiments, the adult human liver stem cells express Oct-4, alpha-fetoprotein, Thy-1, and vimentin.

In another aspect of the invention, adult human liver stem cells are characterized by having a high proliferation potential. In certain embodiments, the adult human liver stem cells can divide at least about 12 times. In other embodiments, the cells can divide at least about 24 times. In certain embodiments, the invention provides adult human liver stem cells that can divide at least about 48 times.

In still another aspect, the invention provides methods of screening compounds for liver toxicity or hepatic function inhibition using the adult liver stem cells, immortalized stem cells or differentiated derivatives of these cells. In this aspect of the invention, the adult liver stem cell, immortalized stem cell or differentiated derivative thereof is first contacted with a test compound. Then, a change in expression of a liver stem cell or differentiated liver stem cell specific gene or activity, or a decrease in cell viability or proliferation potential in the cell contacted with the test compound, as compared to a control cell not contacted with the test compound, is detected. By this method of the invention, a decrease in expression of the gene or activity, or a decrease in cell viability or proliferation potential in the test cell as compared to a control cell indicates that the test compound is toxic to liver cells or inhibits hepatic function.

In particular embodiments of this method, the liver stem cell or differentiated liver stem cell-specific gene or activity monitored is Oct-4, α₁-antitrypsin, γ-glutamyl transpeptidase, alpha-fetoprotein, Thy-1 or vimentin. In other embodiments, the liver stem cell or differentiated liver stem cell-specific gene or activity monitored is P450, glucose-6-phosphatase, catalase, or P-glycoprotein. In still other embodiments, the liver stem cell or differentiated liver stem cell-specific gene or activity monitored is 7-ethoxycoumarin O-de-ethylase, aloxyresorufin O-de-alkylase, coumarin 7-hydroxylase, p-nitrophenol hydroxylase, testosterone hydroxylation, UDP-glucuronyltransferase, glutathione S-transferase, gamma-glutamyl tranpeptidase, or glucose-6-phosphatase. In particular embodiments, the method employs an adult liver stem cell, immortalized stem cell or differentiated derivative thereof obtained by any of the methods described above.

In yet another aspect, the invention provides a method of screening drug compounds for hepatic function therapeutic activity using the adult liver stem cells, immortalized stem cells or differentiated derivatives of these cells. In this aspect of the invention, the adult liver stem cell, immortalized stem cell or differentiated derivative thereof is first contacted with a drug compound. Then, an increase in expression of a liver stem cell or differentiated liver stem cell-specific gene or activity, or an increase in cell viability or proliferation potential in the cell contacted with the test compound as compared to a control cell not contacted with the test compound, is detected. By this method of the invention, an increase in expression of the liver-specific gene or activity, or an increase in cell viability or proliferation potential in the test cell as compared to a control cell indicates that the test compound is cytotoxic to liver cells.

In particular embodiments of this method, the liver stem cell or differentiated liver stem cell-specific gene or activity monitored is P450, glucose-6-phosphatase, catalase, albumin, or P-glycoprotein. In other embodiments, the liver stem cell or differentiated liver stem cell-specific gene or activity monitored is 7-ethoxycoumarin O- de-ethylase, aloxyresorufin O-de-alkylase, coumarin 7-hydroxylase, p-nitrophenol hydroxylase, testosterone hydroxylation, UDP-glucuronyltransferase, glutathione S- transferase, gamma-glutamyl tranpeptidase, or glucose-6-phosphatase. In particular embodiments, the method employs an adult liver stem cell, immortalized stem cell or differentiated derivative thereof obtained by any of the methods described above.

In still another aspect, the invention provides methods of screening compounds for carcinogenicity using the adult liver stem cells of the invention. Then, an increase in the proliferation or other oncogenic character in the adult liver stem cell contacted with the test compound as compared to a control cell not contacted with the test compound, is detected. By this method of the invention, an increase in the proliferation or other neoplastic character in the test cell as compared to a control cell indicates that the test compound is carcinogenic.

In particular embodiments of this method of the invention, the neoplastic character detected may be loss of contact inhibition, the ability to form colonies formation in soft agar, or the presence of an abnormal nuclear morphology. In particular embodiments, the method employs an adult liver stem cell obtained by any of the methods described above.

In yet another aspect, the invention provides differentiated liver tissue which includes one or more cells derived from a human adult clonal liver stem cell obtained by any of the methods described above. In another aspect, the invention provides differentiated liver tissue which includes one or more cells derived from a human adult clonal liver stem cells having the properties described above. In another embodiment, the differentiated liver tissue includes one or more adult liver stem cell derivatives that have been differentiated so that they express one or more hepatocyte-specific functions.

In another aspect, the invention provides a bioartificial liver that includes one or more cells derived from a human adult clonal liver stem cell obtained by any of the methods described above. In another aspect, the invention provides a bioartificial liver, which includes one or more cells derived from a human adult clonal liver stem cells having the properties described above. In a useful embodiment, the bioartificial liver includes one or more adult liver stem cell derivatives that have been differentiated so that they express one or more hepatocyte-specific functions.

In another aspect, the invention provides a method of treating a subject with a liver disease, disorder or dysfunction by providing the subject with a human adult clonal liver stem cell obtained by any of the methods described above. In another aspect, the invention provides a method of treating a subject with a liver disease, disorder or dysfunction by providing the subject with a differentiated liver tissue which includes one or more cells derived from a human adult clonal liver stem cell obtained by any of the methods described above. In yet another aspect, the invention provides a method of treating a subject with a liver disease, disorder or dysfunction by providing the subject with a differentiated liver tissue that includes one or more adult liver stem cell derivatives that have been differentiated so that they express one or more hepatocyte-specific functions.

In yet another aspect, the invention provides a method of treating a subject with a liver disease, disorder or dysfunction by providing the subject with an isolated adult human liver stem cell that is characterized by the lack of expression of gap-junction intercellular communication activity, or by the expression of one or more of the markers Oct-4, alpha-fetoprotein, Thy-1, or vimentin.

In another aspect, the invention provides a method of treating a subject with a liver disease, disorder or dysfunction by providing the subject with a differentiated liver tissue that includes a cell derived from an isolated adult human liver stem cell that is characterized by the lack of expression of gap-junction intercellular communication activity, or by the expression of one or more of the markers Oct-4, alpha-fetoprotein, Thy-1, or vimentin. In another aspect, the subject is provided with a differentiated liver tissue that includes a differentiated adult liver stem cell obtained by any of the methods described above.

In still another aspect, the invention provides a method of treating a subject with a liver disease, disorder or dysfunction by providing the subject with a bioartificial liver that includes a cell derived from an isolated adult human liver stem cell that is characterized by the lack of expression of gap-junction intercellular communication activity, or by the expression of one or more of the markers Oct-4, alpha-fetoprotein, Thy-1, or vimentin. In another aspect, the subject is provided with a bioartificial liver that includes a differentiated adult liver stem cell obtained by any of the methods described above.

The invention also relates to methods of obtaining isolated adult stem cells and propagating isolated adult stem cells, for example, obtaining adult stem cells from adipose tissue. Such cells can also be propagated.

An aspect of the invention relates to a method of isolating adult mammalian stem cells that includes providing a population of dissociated cells comprising stem cells from an adult tissue, culturing the population of dissociated cells in a cell culture medium comprising a low calcium concentration and an effective amount of one or more of N- acetyl-L-cysteine, an antioxidant, and nicotinamide, and allowing adult stem cell colonies to develop in the cell culture medium, thereby yielding a population of adult stem cells. The cell culture medium can be a modified MCDB 153 medium. The low calcium concentration in a cell culture medium of the invention can be less than about 0.3 mM, less than about 0.2 mM, less than about 0.1 mM, about 0.04 mM to about 0.18 mM, about 0.06 mM to about 0.12 mM, about 0.08 mM to about 0.10 mM, or about 0.09 mM. The antioxidant can be vitamin C, for example vitamin C provided as L-ascorbic acid-2-phosphate (e.g., the L-ascorbic acid-2-phosphate can be provided at a concentration of at least about 0.05 mM or at about 0.2 mM). In some embodiments, the antioxidant can be vitamin C, vitamin E, N-acetyl-L-cysteine, resveratrol, coenzyme Q, alpha-lipoic acid, lycopene, bioflavonoids, quercetin, or a combination thereof. The N-acetyl-L-cysteine concentration can be at least about 0.5 mM. In other embodiments, the N-acetyl-L- cysteine concentration is about 2 mM. The nicotinamide concentration can be, e.g., at least about 1 mM or about 5 mM to about 10 mM. The stem cells can be from a mammal such as a primate, e.g., a human. In some embodiments, the isolated adult stem cell population is clonal in origin. In other embodiments, the isolated adult stem cell population is multi-clonal in origin. The isolated adult stem cells may be cultured on tissue culture plastic and form colonies. In another embodiment, the population of isolated adult cells is obtained without the use of feeder cells. The population of isolated adult cells can have a high proliferation potential, for example, wherein the stem cells are mesenchymal stem cells and the proliferation potential is at least 48 cell divisions or the stem cells are liver stem cells and the proliferation potential is about 32 cell divisions. In other embodiments, an adult stem cell can be immortalized by transforming the isolated adult stem cell with an immortalizing gene, e.g., a gene that encodes SV40 large T-antigen, a dominant-negative p53, dominant-negative RB, hTERT, adenovirus E1a, adenovirus E1b, papilloma virus E6, or papilloma virus E7.

In certain embodiments of the invention, the cell culture medium further comprises a growth factor and hormone such as EGF (epidermal growth factor), insulin, hydrocortisone, and 3, 3′, 5-triiodo-D, L-thyronine. In some cases, the cell culture medium includes bovine pituitary extract (BPE), fetal bovine serum (FBS), or both BPE and FBS.

In another embodiment of the invention, the cell culture medium includes at least one of 5 ng/ml of recombinant human EGF, 5 μg/ml of insulin, 74 ng/ml of hydrocortisone, 10 nM 3,3′,5-triiodo-D.L-thyronine, bovine pituitary extract, and 5% to 10% fetal bovine serum. The method can further include culturing an isolated adult stem cell under conditions such that the cell expresses one or more tissue-specific functions. Examples of tissue-specific functions include, without limitation, positive Oil Red 0 staining for lipid vacuoles, Von Kossa staining for calcification of ECM, immunostaining for skeletal myosin expression, and Alcian Blue staining for sulfated proteoglcan accumulation by chondrocytes. In some cases, the isolated adult stem cell of a method described herein is derived from adult adipose tissue and can be induced to form a chondrocyte, myoblast, osteoblast, neuronal cell, or adipocyte. In some embodiments, the adult stem cells are differentiated by contacting them with a medium that includes at least about 0.6 mM calcium. In the methods described herein, the adult stem cell can be derived from adipose tissue and differentiated by contacting the adult stem cell with a chrondrocyte differentiation agent, myoblast differentiation agent, osteoblast differentiation agent, or adipocyte differentiation agent. For example, the differentiation agent can include TGF-β1, L-ascorbate-2-phosphate, and insulin, and the cell differentiates into a chondrocyte; hydrocortisone, and the cell differentiates into a myoblast; or IBMX, dexamethasone, indomethasone, and insulin, and the cell differentiates into an adipocyte.

In another embodiment, the invention includes incubating the isolated adult stem cell in a differentiation agent comprising IBMX, dexamethasone, indomethasone, and insulin for two days, incubating the cell in insulin for one day, repeating the two sets of incubations two additional times, such that the cell differentiates into an adipocyte.

In yet another embodiment, the differentiation agent includes dexamethasone, L-ascorbate-2-phosphate, and β-glycerophosphate, and the cell differentiates into an osteocyte.

In some cases, a differentiated adult stem cell produced as described herein that is expressing one or more tissue-specific functions is provided to a subject in need thereof. The methods of the invention include providing an isolated adult stem cell to a subject in need thereof. The subject is, for example, a mammal such as a human having a disease, disorder, or other dysfunction of adipose tissue, bone, cartilage, nervous system, or muscle. In some cases, the disease, disorder or other dysfunction of the adipose tissue, bone, cartilage, or muscle is, e.g., osteoporosis, bone damage, osteoarthritis, muscular dystrophy, myocardial infarction, cosmetic and reconstructive surgery, or spinal cord injury.

In some embodiments, the invention includes a method of isolating an adult stem cell (e.g., a mesenchymal stem cell such as an adipose stem cell) and wherein the population of dissociated adult cells is cultured in a medium comprising an effective amount of at least two of N-acetyl-L-cysteine, nicotinamide, and an antioxidant; the population of dissociated adult cells is cultured in a medium comprising an effective amount of N-acetyl-L-cysteine, nicotinamide, and an antioxidant; or the population of dissociated adult cells is cultured in a medium comprising about 2 mM N-acetyl-L- cysteine, about 5 mM to about 10 mM nicotinamide, and about 0.2 mM L-ascorbic acid-2-phosphate.

In any of the methods related to isolation of an adult stem cell as described herein, the adult tissue can be adipose tissue and the stem cell can mesenchymal stem cell.

Another aspect of the invention relates to a cell culture medium includes a low calcium ion concentration and an effective amount of one or more of N-acetyl-L- cysteine, nicotinamide, and an antioxidant, for example, an effective amount of at least two of N-acetyl-L-cysteine, nicotinamide, and an antioxidant. In some cases, the cell culture medium includes an effective amount of N-acetyl-L-cysteine, nicotinamide, and an antioxidant, for example, the cell culture medium can include about 2 mM N-acetyl- L-cysteine, about 5 mM to about 10 mM nicotinamide, and about 0.2 mM L-ascorbic acid-2-phosphate. In some cases, the cell culture medium has a low calcium ion concentration is of less than about 0.2 mM, the low calcium ion concentration is about 0.04 mM to about 0.18 mM, the low calcium ion concentration is about 0.08 mM to about 0.10 mM, or the low calcium ion concentration is about 0.09 mM.

In yet another aspect, the invention features a cell culture medium that includes a low calcium ion concentration, an effective amount of one or more agents that promote intracellular glutathione synthesis, an inhibitor of poly ADP-ribose polymerase, and an antioxidant. The cell culture medium can include, for example, about 2 mM N-acetyl-L- cysteine, about 5 mM to about 10 mM nicotinamide, and about 0.2 mM L-ascorbic acid-2-phosphate. In some cases, the cell culture medium described herein has, e.g., a low calcium ion concentration of less than about 0.2 mM, a low calcium ion concentration of about 0.04 mM to about 0.18 mM, a low calcium ion concentration of about 0.08 mM to about 0.10 mM, or a low calcium ion concentration of about 0.09 mM.

In another aspect, the invention includes a cell culture medium for adult human stem cells. The medium includes a calcium ion concentration of not more than about 0.5 mM, or 0 to about 0.5 mM, at least about 1 mM N-acetyl-L-cysteine, at least about 1 mM nicotinamide, and an effective amount of an antioxidant agent, such that the cell culture medium is sufficient for culturing adult human stem cells. In such a medium, the calcium concentration can be, e.g., not more than about 0.2 mM, or 0 to about 0.2 mM, not more than about 0.5 mM, or 0 to about 0.5 mM, or about 0.05 mM to about 0.1 mM. In certain embodiments, the antioxidant is vitamin C. The vitamin C can be provided as, for example, L-ascorbic acid-2-phosphate (e.g., at a concentration of at least about 0.1 mM or a concentration of at least about 0.2 mM). In yet another embodiment, the cell culture medium contains an antioxidant that is selected from e.g., vitamin C, vitamin E, N-acetyl-L-cysteine, resveratrol, or a combination thereof. In certain embodiments, the N-acetyl-L-cysteine concentration in the cell culture medium is at least about 1 mM. In certain embodiments, the nicotinamide concentration in the cell culture medium is at least about 2 mM.

In yet another embodiment, the cell culture medium further includes at least one of EGF, insulin, hydrocortisone, 3,3′,5-triiodo-D.L-thyronine, bovine pituitary extract, or fetal bovine serum, for example, the cell culture medium further includes at least one of 5 ng/ml of recombinant human EGF, 5 μg/ml of insulin, 74 ng/ml of hydrocortisone, 10 nM 3,3′,5-triiodo-D.L-thyronine, 50 μg/ml bovine pituitary extract, and 10% fetal bovine serum.

In some embodiments of the invention, a cell culture medium described herein is used for culturing adult stem cells derived from adipose tissue.

The invention also relates to an adult stem cell obtained by a method described herein, e.g., an adult human stem cell, an isolated clonal or multi-clonal adult human stem cell population obtained by a method described herein, a differentiated cell population derived from adult human stem cell obtained by a method described herein that includes the use of a differentiation agent, for example, an adipocyte, osteocyte, myoblast, neuronal cell, or chondrocyte obtained by a method described herein. In some cases, the adult human stem cell, e.g., does not possess a gap-junction intercellular communication activity, the cell does not express a gap-junction protein such as connexin 26 or connexin 43, the cell expresses at least one of Oct-4 or vimentin.

In certain embodiments, the invention is an adult human stem cell derived from adipose tissue and obtained by a method described herein (an adipose stem cell). For example, the adipose stem cell does not possess a gap-junction intercellular communication activity, the cell does not express a gap-junction protein such as connexin 26 and connexin 43, the cell expresses at least one of Oct-4 or vimentin, or the cell exhibits more than one of these features.

The invention also features an adult human stem cell that is mesenchymal stem cell.

In another aspect, the invention includes an isolated adult human mesenchymal stem cell, such that the cell does not possess a gap-junction intercellular communication activity or the cell does not express a gap-junction protein such as of connexin 26 and connexin 43.

Another aspect of the invention features an isolated adult human mesenchymal stem cell expressing at least one of Oct-4 or vimentin. In some embodiments, the cell or a progeny cell derived from the cell can differentiate into an adipocyte, osteocyte, chondrocyte, neuronal cell, or skeletal muscle cell. The invention also includes an adult human mesenchymal stem cell as described herein such that the cell has a high proliferation potential, e.g., the adult human mesenchymal stem cell can divide at least about 20 times or the cell can divide at least about 32 times. In yet another embodiment, an adult human mesenchymal stem cell as described herein such that a culture of the cell or its progeny has anchorage-dependent growth of e.g., at least about 45% or a culture of the cell or its progeny has anchorage-dependent growth of at least about 56%.

In another aspect, the invention includes methods of using derived stem cells or cells derived from the stem cells (e.g., differentiated cells) for therapeutic activity and carcinogenic activity screens. The invention therefore includes a method of screening compounds for toxicity or inhibition of cellular function. The method includes contacting an adult stem cell, immortalized stem cell, or differentiated derivative thereof obtained by the method of any one of the methods described herein with a test compound, and detecting at least one of a change in a stem cell or differentiated stem cell-specific gene expression or activity, a decrease in cell viability, or a decrease in proliferation potential in the cell contacted with the test compound as compared to a control cell not contacted with the test compound, such that a decrease in a stem cell or differentiated stem cell-specific gene expression or activity, a decrease in cell viability, or a decrease in proliferation potential in the cell in the test cell as compared to a control cell indicates that the test compound is toxic to cells or inhibits cellular function. In some embodiments, the stem cell or its differentiated cell-specific expression or activity is, e.g., Oct-4 expression, vimentin expression, expression of a skeletal myosin, calcification, positive Oil Red 0 staining, or positive Alcian Blue staining. The adult stem cell, immortalized stem cell, or differentiated derivative thereof, can be an isolated adult human stem cell derived from a mesenchymal stem cell (e.g., adipose cell) or an immortalized or differentiated derivative thereof.

In yet another aspect, the invention features a method of identifying a candidate compound that can modulate cellular activity, tissue structure, or tissue function. The method includes contacting an adult stem/precursor cell, immortalized adult stem cell, or differentiated derivative thereof obtained as described herein with a test compound, and detecting an increase in the expression or activity of an adult stem cell gene or gene product, a differentiated adult stem cell-specific gene or gene product, a change in cell viability, or a change in proliferation potential of the cell or its progeny contacted with the test compound as compared to a control cell not contacted with the test compound, such that a change in expression or activity of the adult stem cell, expression or activity of the adult stem cell-specific gene, a change in cell viability, or a change in the proliferation or differentiation potential in the test cell as compared to a control cell indicates that the test compound is a candidate compound for modulating a cellular activity, tissue structure, tissue function. In some cases, the expression or activity of the adult stem cell, expression or activity of the adult stem cell-specific gene, cell viability, proliferation potential, or differentiation potential is increased. In other embodiments, the stem cell or differentiated stem cell-specific gene expression or activity is, e.g., Oct-4 expression, Von Kossa staining, skeletal myosin expression, Alcian Blue staining, or Oil Red O staining. The adult stem cell is an immortalized stem cell or differentiated derivative thereof isolated from an adult human mesenchymal stem cell as described herein, or an immortalized or differentiated derivative thereof.

Another aspect of the invention relates to a method of screening a compound for carcinogenicity. The method includes contacting an adult stem cell, immortalized adult stem cell, or differentiated derivative thereof derived as described herein with a test compound; and detecting an increase in proliferation, or an increase in an oncogenic character in the cell contacted with the test compound as compared to a control cell not contacted with the test compound, such that an increase in the proliferation or other neoplastic character of the test cell compared to a control cell indicates that the test compound is carcinogenic. In some embodiments, the neoplastic character of the cell is loss of contact inhibition, ability to form colonies in soft agar, lifespan extension, or presence of an abnormal nuclear morphology. In the method, the adult stem cell is the isolated adult human mesenchymal stem cell as described herein, e.g., an adipose stem cell.

In yet another aspect, the invention includes a method of screening a compound for cytotoxicity. The invention includes contacting an adult stem cell, immortalized adult stem cell, or differentiated derivative thereof as described herein with a test compound; and detecting at least one of a decrease in cell viability or an increase in apoptosis in the cell contacted with the test compound as compared to a control cell not contacted with the test compound, such that cell death or apoptosis indicates that the test compound is cytotoxic.

The invention also feature methods of using derived stem cells or differentiated cells produced as described herein for production of tissue or a bioartificial system for transplantation. Accordingly, an aspect of the invention is an adipose, muscle, bone, or cartilage tissue that includes tissue-specific differentiated cells derived from an human adult stem cell derived using a method described herein.

In another aspect, the invention features an adipose, muscle, bone, or cartilage tissue that includes tissue-specific differentiated cells derived from an isolated adult human stem cell as described herein.

An additional aspect of the invention is an adipose, muscle, bone, or cartilage tissue comprising tissue-specific differentiated cells derived using a method described herein or an adipose, muscle, bone, or cartilage tissue comprising tissue-specific differentiated cells derived from an isolated adult human stem cell described herein. The invention also features a bioartificial system comprising a cell obtained using a method described herein.

In another aspect, the invention features a method of treating a subject with a disease, disorder, or dysfunction associated with adipose tissue, bone, muscle, or cartilage. The method includes providing the subject with human adult stem cell derived using a method described herein, thereby ameliorating the disease, disorder, or dysfunction. The invention also includes a method of treating a subject with a disease, disorder or dysfunction associated with adipose tissue, bone, muscle, or cartilage comprising providing the subject with a differentiated tissue as described herein, thereby ameliorating the disease, disorder, or dysfunction. Further, the invention relates to a method of treating a subject with a disease, disorder or dysfunction associated with adipose tissue, bone, muscle, or cartilage by a method that includes providing the subject with an isolated adult human stem cell as described herein, thereby ameliorating the disease, disorder, or dysfunction. In some embodiments, the invention includes a method of treating a subject with a disease, disorder or dysfunction associated with adipose tissue, bone, muscle, or cartilage by providing the subject with a differentiated tissue as provided herein, thereby ameliorating the disease, disorder, or dysfunction. In yet another embodiment, the invention includes a method of treating a subject with disorder or dysfunction associated with adipose tissue, bone, muscle, or cartilage by a method that includes providing the subject with a differentiated tissue as provided herein, thereby ameliorating the disease, disorder, or dysfunction.

An aspect of the invention features a method of obtaining isolated adult stem cells. The method includes providing a population of dissociated cells including stem cells from an adult tissue, culturing the population of dissociated cells in a modified MCDB 153 cell culture medium that includes a low calcium concentration and an effective amount of one or more of an agent that promotes intracellular glutathione synthesis, an inhibitor of poly ADP-ribose polymerase, and an antioxidant, and allowing adult stem cell colonies to develop in the cell culture medium, thereby yielding a population of isolated adult stem cells. In some embodiments, the population of dissociated cells is cultured in a medium comprising an effective amount of at least two of an agent that promotes intracellular glutathione synthesis, an inhibitor of poly ADP- ribose polymerase, and an antioxidant. In some cases, the population of dissociated cells is cultured in a medium comprising an effective amount of an agent that promotes intracellular glutathione synthesis, an inhibitor of poly ADP-ribose polymerase, and an antioxidant, for example, the population of dissociated cells are cultured in a medium comprising about 2 mM N-acetyl-L-cysteine, about 5 mM nicotinamide, and about 0.2 mM L-ascorbic acid-2-phosphate. The adult tissue used in the method can be, e.g., adipose tissue.

### 4. BRIEF DESCRIPTION OF THE DRAWINGS

FIG. 1 is a photographic representation of the initial colony that gave rise to the human liver cell line HL1-1. Half of the colony contains actively proliferating smaller cells; the other half contains larger cells with lower cell density. The image is made by superimposing several different pictures of overlapping areas of the colony taken at low magnification (40×).

FIG. 2(a) is a photographic representation of the different areas of the initial HL1-1 colony at higher magnification which reveal that the actively proliferating cells are typical epithelial cells in morphology.

FIG. 2(b) is a photographic representation of the different areas of the initial HL1-1 colony at higher magnification which reveal that the actively proliferating cells are typical epithelial cells in morphology.

FIG. 2(c) is a photographic representation of the different areas of the initial HL1-1 colony at higher magnification which reveal that the actively proliferating cells are typical epithelial cells in morphology whereas most of the larger cells are multinucleated.

FIG. 2(d) is a photographic representation of the different areas of the initial HL1-1 colony at higher magnification which reveal that the actively proliferating cells are typical epithelial cells in morphology whereas most of the larger cells are multinucleated.

FIG. 3(a) is a photographic representation of the initial colonies of HL2 colonies developed in the serum-free K-NAC medium with 5 mM nicotinamide. The colonies shows restricted colony boundary.

FIG. 3(b) is a photographic representation of the initial colonies of HL2 colonies developed in the serum-free K-NAC medium with 5 mM nicotinamide. The colonies shows restricted colony boundary.

FIG. 3(c) is a photographic representation of the initial colonies of HL2 colonies developed in the serum-free K-NAC medium with 5 mM nicotinamide. The colonies shows restricted colony boundary.

FIG. 3(d) is a photographic representation of the initial colonies of HL2 colonies developed in the serum-free K-NAC medium with 5 mM nicotinamide showing that some larger multinucleated cells can be observed in the colony at higher magnification.

FIG. 4(a) is a photographic representations of the HL1-1 cell culture containing serpiginous and cuboidal cell morphologies. The serpiginous cells may divide by symmetrical division (S.D.) resulting in 2 serpiginous cells or by asymmetrical division (AS.D.) resulting in 1 serpiginous cell and 1 cuboidal cell. The cuboidal cell multiplies by symmetrical division.

FIG. 4(b) is a photographic representations of the HL1-1 cell culture containing serpiginous and cuboidal cell morphologies. The serpiginous cells may divide by symmetrical division (S.D.) resulting in two serpiginous cells or by asymmetrical division (AS.D.) resulting in one serpiginous cell and one cuboidal cell. The cuboidal cell multiplies by symmetrical division.

FIG. 4(c) is a photographic representations of the HL1-1 cell culture containing serpiginous and cuboidal cell morphologies. The serpiginous cells may divide by symmetrical division (S.D.) resulting in two serpiginous cells or by asymmetrical division (AS.D.) resulting in one serpiginous cell and one cuboidal cell. The cuboidal cell multiplies by symmetrical division.

FIG. 4(d) is a photographic representations of the HL1-1 cell culture containing serpiginous and cuboidal cell morphologies. The serpiginous cells may divide by symmetrical division (S.D.) resulting in two serpiginous cells or by asymmetrical division (AS.D.) resulting in one serpiginous cell and one cuboidal cell. The cuboidal cell multiplies by symmetrical division.

FIG. 5(a) is a photographic representations of the H L1-1 cells showing that they are capable of anchorage-independent growth (AIG) in K-NAC medium with 5 mM nicotinamide and 10% FBS (AIG frequency 5.5%).

FIG. 5(b) is a photographic representations of the HL1-1 cells showing that they are capable of anchorage-independent growth (AIG) in K-NAC medium with 5 mM nicotinamide and 10% FBS (AIG frequency 5.5%).

FIG. 5(c) is a photographic representations of the HL1-1 cells grown in a modified Eagles MEM with 5 mM nicotinamide and 10% FBS (AIG frequency 3.9%). Note that the colonies appeared dark and, unlike those developed in the K-NAC medium, lost the ability to grow after transfer to plastic surface.

FIG. 5(d) is a photographic representations of the HL1-1 cells grown in a modified Eagles MEM with 5 mM nicotinamide and 10% FBS (AIG frequency 3.9%). Note that the colonies appeared dark and, unlike those developed in the K-NAC medium, lost the ability to grow after transfer to plastic surface.

FIG. 6(a) is a photographic representation of the HL1-1 cells showing that they are deficient in GJIC as assayed by Lucifer Yellow scrape-loading dye transfer technique. Corresponding areas of confluent cells were observed under a fluorescent microscope.

FIG. 6(b) is a photographic representation of the HL1-1 cells showing that they are deficient in GJIC as assayed by Lucifer Yellow scrape-loading dye transfer technique. Corresponding areas of confluent cells were observed under a fluorescent microscope.

FIG. 6(c) is a photographic representation of the HL1-1 cells showing that they are deficient in GJIC as assayed by Lucifer Yellow scrape-loading dye transfer technique. Corresponding areas of confluent cells were observed under a phase contrast microscope.

FIG. 6(d) is a photographic representation of the HL1-1 cells showing that they are deficient in GJIC as assayed by Lucifer Yellow scrape-loading dye transfer technique. Corresponding areas of confluent cells were observed under a phase contrast microscope.

FIG. 7(a) is a photographic representation of the three initial colonies of HL2 cells developed in the serum-free K-NAC medium with 5 mM nicotinamide were found to be deficient in GJIC as assayed by Lucifer Yellow scrape-loading dye transfer technique.

FIG. 7(b) is a photographic representation of the three initial colonies of HL2 cells developed in the serum-free K-NAC medium with 5 mM nicotinamide were found to be deficient in GJIC as assayed by Lucifer Yellow scrape-loading dye transfer technique.

FIG. 7(c) is a photographic representation of the three initial colonies of HL2 cells developed in the serum-free K-NAC medium with 5 mM nicotinamide were found to be deficient in GJIC as assayed by Lucifer Yellow scrape-loading dye transfer technique.

FIG. 7(d) is a photographic representation of the three initial colonies of HL2 cells developed in the serum-free K-NAC medium with 5 mM nicotinamide were found to be deficient in GJIC as assayed by Lucifer Yellow scrape-loading dye transfer technique.

FIG. 7(e) is a photographic representation of the three initial colonies of HL2 cells developed in the serum-free K-NAC medium with 5 mM nicotinamide were found to be deficient in GJIC as assayed by Lucifer Yellow scrape-loading dye transfer technique.

FIG. 7(f) is a photographic representation of the three initial colonies of HL2 cells developed in the serum-free K-NAC medium with 5 mM nicotinamide were found to be deficient in GJIC as assayed by Lucifer Yellow scrape-loading dye transfer technique.

FIGS. 8(a) is a photographic representation of vimentin expression in HL1-1 cells shown by immunostaining.

FIGS. 8(b) is another photographic representation of vimentin expression in HL1-1 cells shown by immunostaining.

FIGS. 8(c) is a photographic representation of nuclear staining by DAPI of the same cells shown in FIG. 8(a).

FIGS. 8(d) is another photographic representation of nuclear staining by DAPI of the same cells shown in FIG. 8(b).

FIG. 8(e) is a photographic representation of a phase contrast image of the same cells shown in FIG. 8(a).

FIG. 8(f) is a photographic representation of a phase contrast image of the same cells shown in FIG. 8(b).

FIG. 9(a) is a photographic representation of α-fetoprotein expression in HL1-1 cells shown by immunostaining.

FIG. 9(b) is another photographic representation of α-fetoprotein expression in HL1-1 cells shown by immunostaining.

FIGS. 9(c) is a photographic representation of nuclear staining by DAPI of the same cells shown in FIG. 9(a).

FIGS. 9(d) is a photographic representation of nuclear staining by DAPI of the same cells shown in FIG. 9(b).

FIG. 9(e) is a photographic representation of a phase contrast image of the same cells shown in FIG. 9(a).

FIG. 9(f) is a photographic representation of a phase contrast image of the same cells shown in FIG. 9(a).

FIG. 10(a) is a photographic representation of Thy-i expression in HL1-1 cells shown by immunostaining.

FIG. 10(b) is a photographic representation of nuclear staining by DAPI of the same cells shown in FIG. 10(a).

FIG. 10(c) is a photographic representation of a phase contrast image of the same cells shown in FIG. 10(a).

FIGS. 11(a) is a photographic representation of cytokeratin 7 expression in HL1-1 cells shown by immunostaining.

FIGS. 11(b) is another photographic representation of cytokeratin 7 expression in HL1-1 cells shown by immunostaining.

FIGS. 11(c) is a photographic representation of nuclear staining by DAPI of the same cells shown in FIG. 11(a).

FIGS. 11(d) is a photographic representation of nuclear staining by DAPI of the same cells shown in FIG. 11(b).

FIG. 11(e) is a photographic representation of a phase contrast image of the same cells shown in FIG. 11(a).

FIG. 11(f) is a photographic representation of a phase contrast image of the same cells shown in FIG. 11(a).

FIG. 12(a) is a photographic representation of cytokeratin 8 expression in HL1-1 cells shown by immunostaining.

FIG. 12(b) is a photographic representation of nuclear staining by DAPI of the same cells shown in FIG. 12(a).

FIG. 12(c) is a photographic representation of a phase contrast image of the same cells shown in FIG. 12(a).

FIG. 13(a) is a photographic representation of cytokeratin 18 expression in HL1-1 cells shown by immunostaining.

FIG. 13(b) is a photographic representation of nuclear staining by DAPI of the same cells shown in FIG. 13(a).

FIG. 13(c) is a photographic representation of a phase contrast image of the same cells shown in FIG. 13(a).

FIGS. 14(a) is a photographic representation of cytokeratin 19 expression in HL1-1 cells shown by immunostaining.

FIGS. 14(b) is another photographic representation of cytokeratin 19 expression in HL1-1 cells shown by immunostaining.

FIGS. 14(c) is a photographic representation of nuclear staining by DAPI of the same cells shown in FIG. 14(a).

FIGS. 14(d) is a photographic representation of nuclear staining by DAPI of the same cells shown in FIG. 14(b).

FIG. 14(e) is a photographic representation of a phase contrast image of the same cells shown in FIG. 14(a).

FIG. 14(f) is a photographic representation of a phase contrast image of the same cells shown in FIG. 14(a).

FIG. 15(a) is a photographic representation of vimentin expression in HL3-2 cells shown by immunostaining.

FIG. 15(b) is a photographic representation of nuclear staining by DAPI of the same cells shown in FIG. 15(a).

FIG. 15(c) is a photographic representation of a phase contrast image of the same cells shown in FIG. 15(a).

FIG. 16(a) is a photographic representation of α-fetoprotein in HL3-2 cells shown by immunostaining.

FIG. 16(b) is a photographic representation of nuclear staining by DAPI of the same cells shown in FIG. 16(a).

FIG. 16(c) is a photographic representation of a phase contrast image of the same cells shown in FIG. 16(a).

FIG. 17(a) is a photographic representation of Thy-1 in HL3-2 cells shown by immunostaining.

FIG. 17(b) is a photographic representation of nuclear staining by DAPI of the same cells shown in FIG. 17(a).

FIG. 17(c) is a photographic representation of a phase contrast image of the same cells shown in FIG. 17(a).

FIG. 18(a) is a photographic representation of a Western blot showing vimentin expression of HL1-1 cells. Lane 1 and 2 are duplicates of HL1-1 cell extract, while lane 3 is the human fibroblast (MSU-2) control.

FIG. 18(b) is a photographic representation of a Western blot showing α-fetoprotein expression of HL1-1 cells. Lane 1 and 2 are duplicates of HL1-1 cell extract, while lane 3 is the human fibroblast (MSU-2) control.

FIG. 19(a) is a photographic representation of HL1-1 cells after growing in modified Eagle's MEM for 4-5 days, which shows that they become competent in GJIC as assayed by the scrape-loading dye transfer technique.

FIG. 19(b) is a photographic representation of a phase contrast image of the cells shown in FIG. 19(a).

FIG. 20(a) is a photographic representation of HL1-1 cells cultured in the K- NAC medium, which shows that they form “bridges” between cell micromasses. Each aggregate contains 1×10⁵ cells plated in 24-well plate.

FIG. 20(b) is another photographic representation of HL1-1 cells cultured in the K-NAC medium, which shows that they form “bridges” between cell micromasses. Each aggregate contains 1×10⁵ cells plated in 24-well plate.

FIG. 21(a) is a photographic representation of albumin expression by L1SV1A1 cells treated with hepatocyte growth factor (20 ng/ml) for 33 days as studied by immunostaining.

FIG. 21(b) is a photographic representation of nuclear staining by DAPI of the same cells shown in FIG. 21(a).

FIG. 21(c) is a photographic representation of a phase contrast image of the same cells shown in FIG. 21(a).

FIG. 22(a) is a photographic representation of vimentin expression in Mahlava cells shown by immunostaining.

FIG. 22(b) is a photographic representation of nuclear staining by DAPI of the same cells shown in FIG. 22(a).

FIG. 22(c) is a photographic representation of a phase contrast image of the same cells shown in FIG. 22(a).

FIG. 23(a) is a photographic representation of α-fetoprotein expression in Mahlava cells shown by immunostaining.

FIG. 23(b) is a photographic representation of nuclear staining by DAPI of the same cells shown in FIG. 23(a).

FIG. 23(c) is a photographic representation of a phase contrast image of the same cells shown in FIG. 23(a).

FIG. 24(a) is a photographic representation of Thy-I expression in Mahlava cells shown by immunostaining.

FIG. 24(b) is a photographic representation of nuclear staining by DAPI of the same cells shown in FIG. 24(a).

FIG. 24(c) is a photographic representation of a phase contrast image of the same cells shown in FIG. 24(a).

FIGS. 25(a) is a photographic representation of the hepatoma cell line, Mahlava, showing that it is deficient in GJIC as shown by the scrape-loading dye transfer technique.

FIGS. 25(b) is a photographic representation of the same cells shown in FIG. 25(a).

FIG. 26 is a diagrammatic representation showing that telomerase activity is activated in the L1SV1A1 cell line. hTEER mRNA using the LightCycler TeloTAGGGhTERT was quantitatively detected using a LightCycler instrument and quantification kit. The AGS is a human gastric epithelial cell line for positive control. The NTC is the no template control.

FIG. 27(a) is a pair of photographic representations of mesenchymal stem cells cultured under conditions that include low calcium.

FIG. 27(b) is a set of photographic representations of dividing mesenchymal stem cells.

FIG. 28 is pair of photographic representations of mesenchymal stem/precursor cells cultured on a plastic surface to test for anchorage independent growth.

FIG. 29 is a set of photographic representations of cell cultures that have some serpiginous cells that were assayed for gap junctional intercellular communication using the scrape loading/Lucifer yellow dye transfer method. Left panels are fluorescent images and right panels are phase-contrast images of the corresponding fluorescent image.

FIG. 30 is a pair of graphical representation depicting the results of experiments in which the cpdl of mesenchymal stem/precursor cells (isolated and propagated using methods described herein) was assayed (bottom panel). The top panel is a representation of a bar graph reproduced from Tissue Engineering 7: 211-228, 2001, illustrating the cpdl for mesenchymal stem cells isolated and propagated using methods described in the reference.

FIG. 31(a) is a set of photographic representations depicting the results of experiments in which mesenchymal stem/precursor cells derived from adipose tissue were differentiated into adipocytes under adipogenic differentiation conditions. The top two panels and the left panel are cells cultured in differentiation medium. Bottom right is a control cultured without differentiation medium. Lipid vacuoles can be seen in the cultures.

FIG. 31 (b) is a set of photographic representations depicting the results of experiments in which mesenchymal stem/precursor cells derived from adipose tissue were differentiated into adipocytes under adipogenic conditions (bottom panels) or control cells cultured without differentiation medium (top right panel) and Oil Red O stained.

FIG. 32 (a) is a set of photographic representations depicting the results of experiments in which mesenchymal stem/precursor cells derived from adipose tissue were differentiated into osteocytes under osteogenic differentiation conditions. Cultures depicted in the top panels and bottom right panel were grown under differentiation conditions. The culture depicted in the bottom left panel was grown without induction medium (control).

FIG. 32(b) is a set of photographic representations depicting the results of experiments in which mesenchymal stem/precursor cells derived from adipose tissue were differentiated into osteocytes under osteogenic differentiation conditions and Von Kossa stained. Cultures depicted in the bottom panels were grown under differentiation conditions. The culture depicted in the right top panel depicts a control culture that was grown without differentiation induction.

FIG. 33(a) is a graphical representation depicting the results of quantitative assays measuring calcified ECM in adipocyte stem cell cultures grown under osteogenic induction conditions and control cultures grown without induction.

FIG. 33 (b) is a graphical representation depicting the results of quantitative assays measuring calcium in culture medium in adipocyte stem cell cultures grown under osteogenic induction conditions and control cultures grown without induction.

FIG. 34 is a photographic representation depicting Alcian blue stained cultures of mesenchymal stem/precursor cells cultured in chondrocyte induction medium (bottom panels) or control cells that were not cultured in the induction medium (right top panel).

FIG. 35 is a graphical representation depicting the results of experiments in which adipose stem cells were cultured in osteogenic induction medium (osteogenic differentiation), osteogenic differentiation medium plus lovastatin (0.2 μM), or in mesenchymal stem cell medium (control) and the amount of total calcium assayed (mg/plate).

### 5. DETAILED DESCRIPTION OF THE INVENTION

The patent and scientific literature referred to herein establishes knowledge that is available to those of skill in the art. The issued U.S. patents, allowed applications, published foreign applications, and references, including GenBank database sequences, that are cited herein are hereby incorporated by reference to the same extent as if each was specifically and individually indicated to be incorporated by reference. This Application incorporates by reference in their entireties the teachings of U.S. Provisional Application No. 60/559,747, filed Apr. 6, 2004, entitled “Oct-4 and GJIC Expression as Markers for Adult Human Stem Cells and Metastatic Cells”, and U.S. Provisional Application No. 60/548,212, filed Feb. 27, 2004, also entitled “Oct-4 and GJIC Expression as Markers for Adult Human Stem Cells and Metastatic Cells”.

5.1. General

The invention is based in part upon a novel method of culturing adult human stem cells (for example, liver stem/progenitor cells or mesenchymal stem/progenitor cells) that are derived from adult tissues (e.g., liver or fat) that allows for the growth and isolation of clonal stem cells that can be further propagated or differentiated into mature cell types such as hepatocytes, adipocytes, osteocytes, or chondrocytes. The invention provides a method for the isolation and culture of human adult stem cells (e.g., liver stem cells or mesenchymal stem cells) for sustained growth. The invention further provides human adult stem cells such as liver stem cells or mesenchymal stem cells, including immortalized lines of such stem cells, as well as differentiated cells such as hepatocytes, liver tissues, adipocytes, osteoblasts, chondrocytes, muscle (e.g., myocytes), pancreatic, and neuronal cells that are derived from such stem cells.

Stem cells are undifferentiated cells with the capacity for unlimited or prolonged self-renewal and the ability to give rise to differentiated cells. Different adult stem cells may exhibit different specific gene expression. For example, liver oval cells express vimentin, α-fetoprotein, Thy-1, and the hematopoietic stem cell markers, CD34 and SCF/c-kit, which are not expressed in hepatocytes or bile duct cells (Alison (1998) Curr. Opin. Cell Biol. 10:710-715). Therefore, the expression of vimentin and alpha- fetoprotein has been collectively termed the “oval cell phenotypes” (Alison et al. (1997) J. Hepatol. 26:343-352). Liver oval cells are located in the smallest unit of the biliary tree (i.e., the canals of Herring or Cholangioles), and are believed to represent the progeny of pluripotential liver stem cells that are capable of generating hepatic lineages (Alison (1998) Curr. Opin. Cell Biol. 10:710-715; Evarts et al. (1987) Carcinogenesis 8:1737-1740; Thorgeirsson (1993) Am. J. Pathol. 142:1331-1333; Coleman et al. (1997) Am. J. Pathol. 151:353-359; Yasui et al. (1997) Hepatol. 25:329-334). Although adult liver stem or progenitor cells are thus potentially identifiable histologically, the instant invention provides means for their isolation and propagation. More generally, the instant invention provides new cell culture methods that allow for the efficient isolation and growth of human adult stem cells from different tissues.

The invention exploits, in part, the finding that the transcription factor Oct-4, previously shown to be exclusively expressed in pluripotent early embryo stem cells, embryonic stem cells, and undifferentiated tumor cells is expressed in several types of adult human stem cells including those derived from the liver and from adipose tissue. Furthermore, the invention uses morphological characteristics to identify adult stem cells. Multipotential pancreatic cells and oligodendrocyte precursor cells can be recognized by their serpiginous-shaped morphology (Zulewski et al. (2001) Diabetes 50:521-533; Tang et al. (2001) Science 291:872-875), while other stem cells are characterized by their smaller size (blast-like cells) than corresponding mature or differentiated cell type (Sigal et al. (1992) Am. J. Physiol. 263:G139-G148). The deficiency in gap junctional intercellular communication also appears to be a common phenotype of stem cells (Chang et al. (1987) Cancer Res. 47:1634-1645; Kao et al. (1995) Carcinogenesis 16:531-538; Matic et al. (2002) J. Invest. Dermatol. 118:110-116; Grueterich et al. (2002) Arch. Ophthalmol. 120:783-790). This feature can be used to identify stem cells cultured as described herein.

The invention also provides for immortalized adult stem cells, e.g., human liver stem cells, mesenchymal stem cells, and differentiated derivatives of these cells. Methods for immortalizing cells are known in the art. For example, human adult and fetal hepatocytes can be immortalized by Simian virus 40 (SV40) large T-antigen (Pfeifer et al. (1993) Proc. Natl. Acad. Sci. U.S.A 90:5123-5127), and the catalytic subunit of the telomerase hTERT (Wege et al. (2003) Gastroenterol. 124:432444), respectively. Such immortalized cells have been shown to express hepatocytes genes while remaining non- tumorigenic. Bipotent epithelial liver stem cells derived from monkey fetal liver can be immortalized by SV40 large T-antigen at low frequency (1 in 144 transformed clones) (Allain et al. (2002) Proc. Natl. Acad. Sci. U.S.A 99:3639-3644).

In some cases, the invention relates to the culture and use of stem cells derived from any adult tissue (e.g., adipose tissue) that harbors such cells. An advantage of using adipose tissue as a source of stem cells is that adipose tissue can be relatively easily obtained from the body of a person who is to receive the stem cells or differentiated derivatives thereof for autologous cell therapy. The use of autologous cells avoids problems associated with immune incompatibility and transfer of disease from donor to recipient. In addition, adipose cells can be collected with relative ease (e.g., from lipoaspirates) and in relatively large amounts. Allogenic donors who might otherwise be unwilling to undergo harvesting of stem cells from other tissues may be more likely to agree to donate adipose tissue. Thus, the use of adipose tissue as a source of stem cells can expand the pool of willing donors.

5.2 Definitions

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “adult” as used herein, is meant to refer to any non-embryonic mammalian organism. For example the term “adult liver stem cell,” refers to a liver stem cell, other than that obtained from an embryo (e.g., a stem cell obtained from a postpartum viable offspring).

The term “alpha-fetoprotein” refers to both glycosylated and non-glycosylated proteins from the serum of vertebrate embryos, which likely serve as albumins. For example, human alpha-fetoprotein includes secreted forms of the human alpha- fetoprotein precursor specified by GenBank Accession No. AAH27881.

The term “antibody” as used herein refers to both polyclonal and monoclonal antibody. The term encompasses not only intact immunoglobulin molecules, but also such fragments and derivatives of immunoglobulin molecules (such as single chain Fv constructs, diabodies, and fusion constructs) that retain a desired antibody binding specificity, as may be prepared by techniques known in the art.

The term “antioxidant” refers to any substance, often an organic compound, that opposes oxidation or inhibits reactions brought about by dioxygen or peroxides. Usually the antioxidant is effective because it can itself be more easily oxidized than the substance protected. The term includes components that can trap free radicals, such as α-tocopherol (vitamin E), thereby breaking the chain reaction that normally leads to extensive biological damage. As used herein, the term “antioxidant” includes biological molecules that serve as natural free-radical scavengers intracellularly including ascorbate (vitamin C), glutathione and NAD⁺. “Antioxidant” further includes natural and synthetic compounds commonly added to protect labile compounds during storage or incubation (e.g., di-tert-butyl-p-cresol, and quinhydrone).

“Calcium” refers to soluble, biologically active calcium ion (Ca⁺²). The term “low calcium concentration” means a concentration of calcium ion (Ca⁺²) sufficiently low (e.g., less than about 0.2 mM), to allow propagation, while preventing differentiation, of an adult liver stem cell.

The terms “cell culture” and “culture” encompasses the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “conditioned medium” is one prepared by culturing a first population of cells in a medium, and then harvesting the medium. The conditioned medium (along with anything secreted into the medium by the cells) may then be used to support the growth of a second population of cells.

“Connexin” refers to a principal protein component of a connexon. Connexon proteins typically contain four putative membrane-spanning alpha-helices, and six connexins typically make up each connexon structure. For example, the human connexins include human connexin 26 (see GenBank Accession No. NP_{—}003995), and connexin 43 (see GenBank Accession No. Q9Y6H8).

“Feeder cells” or “feeders” are terms used to describe cells of one type that are co- cultured with cells of a second type, to provide an environment in which the cells of the second type can be maintained, and perhaps proliferate. The feeder cells can be from a different species than the cells they are supporting. For example, certain stem cells can be supported by mouse embryonic fibroblasts (from a primary culture or a telomerized line) or human fibroblast-like or mesenchymal cells. Typically (but not necessarily), feeder cells are inactivated by irradiation or treatment with an anti-mitotic agent such as mitomycin C, to prevent them from outgrowing the cells they are supporting.

The term “gap junction” refers to any specialized area of a plasma membrane of apposed vertebrate cells where the membranes are 2-4 nm apart and where each membrane is penetrated by one or more connexons, such that the gap junction bridges the extracellular space and provides open means of communication between the cytoplasm of one cell and that of the other cell.

The term “hepatocyte,” as used herein, means a predominant cell of the liver responsible for the synthesis, degradation and storage of a wide range of substances within the liver. Hepatocytes are the site of synthesis of plasma proteins, other than antibodies, and are the site of storage of glycogen. Within the liver, but not necessarily when propagated in cell culture, hepatocytes are arranged in folded sheets facing blood- filled spaces called sinusoids.

The terms “differentiation agent” and “maturation factor” represent a collection of one or more compounds that are used in differentiation or maturation methods that can be used in preparing and maintaining the differentiated cells (e.g., hepatocyte cells, adipoblasts, adipocytes, osteoblasts, osteocytes, myoblasts, myocytes, chondroblasts, chrondrocytes, neuroblasts, or neuronal cells) of the invention. These agents are further described and exemplified in the sections that follow and as incorporated by reference. The use of a term with a specific cell type (e.g., “hepatocyte differentiation agent”) refers to an agent that can be used to prepare or maintain the specific cell type.

The term “ingredient” refers to any compound, whether of chemical or biological origin, that can be used in cell culture media to maintain or promote the growth or proliferation of cells. The terms “component,” “nutrient” and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “isolated,” used in reference to a single cell or clonal cell cluster, e.g., a stem cell or hepatocyte or clonal colony thereof, means that the cell is substantially free of other nonclonal cells or cell types or other cellular material with which it naturally occurs in the tissue of origin (e.g., liver or adipose tissue). A sample of stem cells is “substantially pure” when it is at least 60%, or at least 75%, or at least 90%, and, in certain cases, at least 99% free of cells other than cells of clonal origin. Purity can be measured by any appropriate method, for example, by fluorescence-activated cell sorting (FACS).

The term “nicotinamide,” as used herein, refers to pyridine-3-carboxamide, the amide of nicotinic acid, also referred to as niacinamide. Nictotinamide is a member of the B complex of vitamins and is equivalent to nicotinic acid. Nicotinamide is a precursor of nicotinamide-adenine dinucleotide (NAD⁺ or NAD), as well as of nicotinamide-adenine dinucleotide phosphate (NADP⁺ or NADP). NAD and NADP are specific coenzymes in numerous cellular oxidoreductase enzyme reactions.

The term “Oct” refers to a family of genes and the proteins they encode, which are transcription factors for eukaryotic RNA polymerase II promoters. An Oct protein contains a POU domain and a leucine zipper domain that contributes to binding to octamer sequences (eight-base sequence elements that are common in eukaryotic promoters and that have the consensus sequence ATTTGCAT). For example, “Oct-4” includes the gene encoding human Oct-4 factor corresponding to GenBank Accession No. Q01860. As described herein, certain stem cells derived from an adult tissue can be identified, at least in part, by their ability to express an Oct (i.e., nucleic acid or protein). “Stem cell,” as used herein, encompasses any member of the various groups of reserve cells whose role is to replace cells that are destroyed during the normal life of the animal, e.g., hepatocytes, blood cells, epithelial cells, spermatogonia, adipocytes, osteoblasts, chondrocytes, and skin cells. Stem cells may divide without limit; after division, the stem cell may remain as a stem cell or proceed to terminal differentiation (e.g., to become a mature hepatocyte). By “liver stem cell” is meant an undifferentiated cell derived from liver that can differentiate into a mature functional hepatocyte and/or bile duct cell. A liver stem cell can also transdifferentiate into a non-liver cell type such as a pancreatic cell. A mesenchymal stem cell is derived from a mesenchymal tissue (e.g., adipose tissue) and can differentiate into a variety of cell types, including, but not limited to, those described herein.

The term “Thy-1” refers to a differentiation antigen that is present on, for example, T lymphocytes and also occurs in the brain. Thy-1 is a GPI-anchored membrane glycoprotein of the immunoglobulin superfamily, with a simple structure homologous to the variable region of an immunoglobulin (e.g., the human Thy-i corresponding to GenBank Accession No. AAA61180).

The term “vimentin” refers to a protein found in class III intermediate filaments in, for example, mesenchymal and other nonepithelial cells, and in the Z disk of skeletal and cardiac muscles cells (including, e.g., the human vimentin protein corresponding to GenBank Accession No. CAA79613). Vimentin is a phosphoprotein, phosphorylation being enhanced during cell division.

5.3 Cell Culture Media and Methods

The invention includes novel cell culture media and formulations. In certain instances, the novel compositions include improvements or variations of known media. For example, the invention features a specialized cell culture medium for the culture of adult stem cells. This cell culture medium is useful for obtaining and propagating e.g., isolated clonal adult stem cells from human adult liver tissue samples (adult liver stem cells) or adipose tissue (adipose stem cells), which can be from an adult. In general, the culture medium is a medium (e.g., a modified MCDB 153 medium) containing a low concentration of calcium, which is suitable for growing mesenchymal stem cells and precursor cells before induction of differentiation. The medium can be supplemented, e.g., with L-ascorbate-2-phosphate and N-acetyl-L-cysteine. Other modifications and supplements are described below.

In one aspect, the adult stem cell culture media of the invention is formulated to prevent stem cell differentiation. High calcium concentration in cell culture media seems to favor cell differentiation in particular instances such as the development of human epidermal keratinocytes into mature skin cells (see, e.g., Rheinwald et al. (1975) Cell 6: 331-43; and Yuspa et al. (1981) Nature 293: 72-74). In contrast, the invention includes media containing relatively low concentrations of calcium to help prevent differentiation of adult stem cells (i.e., compared to typical cell media used to propagate, for example, fibroblasts). For example, a typical cell medium such as Dulbecco's modified MEM (DMEM) has a calcium ion concentration of about 1.8 mM. A low calcium ion medium described herein is less than about 1.8 mM in all instances where low calcium is a feature, and can be less than about 0.9 mM, less than about 0.5 mM, less than about 0.4 mM, less than about 0.3 mM, less than about 0.2 mM, or less than about 0.1 mM. A low calcium ion medium can further have a calcium concentration of about 0.03-0.3 mM, about 0.04-0.20 mM, about 0.06-0.12 mM or about 0.08-0.10 mM. In certain instances, a calcium ion concentration of about 0.1 or 0.09 mM is useful. The calcium ion can be supplied as any biocompatible salt such as calcium chloride (CaCl₂), calcium carbonate (CaCO₃) or calcium sulfate (CaSO₄). Furthermore, other components of cell media, or analytes that are compatible with cell media, may also be adjusted to achieve the same affect in disfavoring cell differentiation while allowing continued stem cell growth and division. These equivalent “alterations” to cell media to disfavor cell differentiation are known in the art, or may be elucidated, or confirmed to have such an effect with adult stem cells, using routine experimentation.

In another aspect, the adult stem cell culture media of the invention are formulated to prevent or reduce oxidation by including one or more antioxidant compounds. For example, vitamin C (supplied as in the stable form of L-ascorbic acid-2-phosphate or in any other form) may be used. Furthermore, N-acetyl-L-cysteine, a potent antioxidant, which is also a precursor of L-cysteine and which thereby promotes the intracellular synthesis of glutathione (see below), may be a useful antioxidant to include in the stem cell culture media formulations of the invention. Other antioxidants that can be included are vitamin E (e.g., D-alpha-tocopherol), resveratrol (a plant phytoalexin derived from, for example, grapes), coenzyme Q, alpha-lipoic acid, lycopene, bioflavonoids, and quercetin. The form and concentration of each such antioxidant is known in the art or can be discerned with routine testing. For example, L-ascorbic acid-2-phosphate may be used at a concentration of about 20 μM to about 2 mM (e.g., at 0.2 mM), and N-acetyl-L-cysteine may be used at a concentration of about 0.2 to about 20 mM (e.g., at 2 mM).

In yet another aspect, the adult stem cell culture media of the invention is formulated to promote intracellular glutathione synthesis. For example, as described above, N-acetyl-L-cysteine is both a powerful antioxidant and a precursor to L-cysteine, which promotes intracellular glutathione synthesis. Accordingly, N-acetyl-L-cysteine is useful for the dual effect on intracellular glutathione levels and as an antioxidant. Other means for increasing intracellular glutathione synthesis are known in the art or can be discerned with routine testing and are within the ambit of the invention. For example, the addition of external oxidized glutathione (GSSG) at concentrations in the range of 50 to 500 μM was found to increase the level of intracellular glutathione in isolated rat myocytes (see Guarnieri et al. (1987) Biochem. Biophys. Res. Commun. 147: 658-65). Additionally, the anti-rheumatic drug, KE-298 (2-acetylthiomethyl-4-(4-methylphenyl)-4-oxobutanoic acid) and its active metabolite; KE-758 (2-mercaptomethyl-4-(4-methylphenyl)-4-oxobutanoic acid) may have a similar effect on cells (see Sugimoto et al. (2002) Mol. Immunol. 38: 793-9). The form and concentration of each such glutathione level-stimulating agent is known in the art or may be discerned with routine testing. For example, N-acetyl-L-cysteine can be used at a concentration of about 0.2 to about 20 mM (e.g., at 2 mM).

In still another aspect, an adult stem cell culture medium of the invention includes nicotinamide, which is known to function as an inhibitor of poly-ADP-ribose polymerase. Other means for inhibiting poly-ADP-ribose polymerase activity are known in the art or may be discerned with routine testing and are within the ambit of the invention. For example, purines, including hypoxanthine, inosine, and adenosine, have been shown to inhibit poly-ADP-ribose polymerase (also known as PARP) both in vivo and in vitro in a dose-dependent manner (see Virag and Szabo (2001) FASEB J. 15: 99-107). 1,5-Dihydroxy-isoquinoline and 3-aminobenzamide are also potent PART inhibitors. Furthermore, the structures and pharmacological actions of various PARP inhibitors have been described (see Southan and Szabo (2003) Curr. Med. Chem. 10: 321-40). The form and concentration of each such PARP inhibitor is known in the art or can be discerned with routine testing. For example, nicotinamide can be used at a concentration of about 0.5 mM to about 50 mM (e.g., at 5 mM).

The concentrations and other ingredients in a formulation of standard cell culture medium are well known to those of ordinary skill in the art. (See Methods For Preparation of Media, Supplements and Substrate For Serum-Free Animal Cell Culture, Allen R. Liss, N.Y. (1984), which is incorporated by reference herein in its entirety). These media formulations can be adapted to the invention through appropriate modification of the critical features of the medium, such as those described above.

In general, cell culture media provide the nutrients necessary to maintain and grow cells in a controlled, artificial, and in vitro environment. Characteristics and compositions of the cell culture media vary depending on the particular cellular requirements. Important parameters include osmolarity, pH, and nutrient formulations.

Media formulations have been used to cultivate a number of cell types including animal, plant, and bacterial cells. Cells cultivated in culture media catabolize available nutrients and produce useful biological substances such as monoclonal antibodies, hormones, growth factors and the like. Such products have therapeutic applications and, with the advent of recombinant DNA technology, cells can be engineered to produce large quantities of these products.

Cell culture media formulations have been well documented in the literature and a number of media are commercially available. In early cell culture work, media formulations were based upon the chemical composition and physicochemical properties (e.g., osmolality, pH, etc.) of blood and were referred to as “physiological solutions” (Ringer, J. Physiol. (1880) 3: 380-393; Waymouth (1965) In: Cells and Tissues in Culture, Vol. 1, Academic Press, London, pp. 99-142; Waymouth (1970) In Vitro 6:109-127). Cells in different tissues of the mammalian body are exposed to different microenvironments with respect to oxygen/carbon dioxide partial pressure and concentrations of nutrients, vitamins, and trace elements; accordingly, successful in vitro culture of different cell types will often require the use of different media formulations. Typical components of cell culture media include amino acids, organic and inorganic salts, vitamins, trace metals, sugars, lipids and nucleic acids, the types and amounts of which may vary depending upon the particular requirements of a given cell or tissue type.

Typically, cell culture media formulations are supplemented with a range of additives, including undefined components such as fetal bovine serum (FBS) (5-20% v/v) or extracts from animal embryos, organs or glands (0.5-10% v/v). While FBS is the most commonly applied supplement in animal cell culture media, other serum sources are also routinely used, including newborn calf, horse, and human. Organs or glands that have been used to prepare extracts for the supplementation of culture media include submaxillary gland (Cohen (1961) J. Biol. Chem. 237: 1555-1565), pituitary (Peehl and Ham (1980) In Vitro 16: 516-525; see U.S. Pat. No. 4,673,649), hypothalamus (Maciag, et al. (1979) Proc. Natl. Acad. Sci. USA 76: 5674-5678; Gilchrest, et al. (1984) J. Cell. Physiol. 120: 377-383), and brain (Maciag, et al. (1981) Science 211: 1452-1454). These types of chemically undefined supplements serve several useful functions in cell culture media (see Lambert, et al. (1985) In: Animal Cell Biotechnology, Vol. 1, Spier et al., Eds., Academic Press, New York, pp. 85-122 (1985)). For example, these supplements (1) provide carriers or chelators for labile or water-insoluble nutrients; (2) bind and neutralize toxic moieties; (3) provide hormones and growth factors, protease inhibitors and essential, often unidentified or undefined low molecular weight nutrients; and (4) protect cells from physical stress and damage. Thus, serum or organ/gland extracts are commonly used as relatively low-cost supplements to provide an optimal culture medium for the cultivation of animal cells.

A number of so-called “defined” media, which avoid the use of animal serum (and/or animal extracts), have also been developed. These media, which often are specifically formulated to support the culture of a single cell type, contain no undefined supplements and instead incorporate defined quantities of purified growth factors, proteins, lipoproteins and other substances usually provided by the serum or extract supplement. Since the components (and concentrations thereof) in such culture media are precisely known, these media are generally referred to as “defined culture media.” Often used interchangeably with “defined culture media” is the term “serum-free media” or “SFM.” A number of SFM formulations are commercially available, such as those designed to support the culture of endothelial cells, keratinocytes, monocytes/macrophages, fibroblasts, chondrocytes, or hepatocytes, which are available from GIBCO/LTI (Gaithersburg, Md.). The distinction between SFM and defined media, however, is that SFM are media devoid of serum, but not necessarily of other undefined components such as organ/gland extracts. Indeed, several SFM that have been reported or that are available commercially contain such undefined components, including several formulations supporting in vitro culture of keratinocytes (Boyce and Ham (1983) L Invest. Dermatol. 81:33; Wille et al. (1984) J. Cell. Physiol. 121:31; Pittelkow and Scott (1986) Mayo Clin. Proc. 61:771; Pirisi et al. J. Virol. 61: 1061; Shipley and Pittelkow (1987) Arch. Dermatol. 123: 1541; Shipley et al. (1989) J. Cell. Physiol. 138: 511-518; Daley et al. (1990) FOCUS (GIBCO/LTI) 12:68; and U.S. Pat. Nos. 4,673,649 and 4,940,666).

Defined media generally provide several distinct advantages to the user. For example, the use of a defined medium facilitates the investigation of the effects of a specific growth factor or other medium component on cellular physiology, which may be masked when the cells are cultivated in serum- or extract-containing media. In addition, defined media typically contain much lower quantities of protein (indeed, defined media are often termed “low protein media”) than those containing serum or extracts, rendering purification of biological substances produced by cells cultured in defined media far simpler.

Some extremely simple defined media, which consist essentially of vitamins, amino acids, organic and inorganic salts and buffers, have been used for cell culture. Such media (often called “basal media”), however, are usually seriously deficient in the nutritional content required by most animal cells. Accordingly, most defined media incorporate into the basal media additional components to make the media more nutritionally complex, but to maintain the serum-free and low protein content of the media. Non-limiting examples of such components include serum albumin from bovine (BSA) or human (HSA); certain growth factors derived from natural (animal) or recombinant sources such as EGF or FGF; lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements (reviewed by Waymouth (1984) In: Cell Culture Methods for Molecular and Cell Biology, Vol. 1: Methods for Preparation of Media, Supplements, and Substrata for Serum-Free Animal Cell Culture, Barnes et al., eds., New York: Alan R. Liss, Inc., pp. 23-68; and by Gospodarowicz, Id., at pp 69-86).

In some instances, a specialized medium of the invention, adapted for propagation of stem cells, and differentiated or immortalized derivatives of such cells, is a modified formulation of a medium developed for the propagation of keratinocytes.

Keratinocytes are the specialized epithelial cells found in the epidermis of the skin. In the upper, cornified layers of the skin (those exposed to the environment), the cytoplasm of the keratinocytes is completely replaced with keratin and the cells are dead. The keratinocytes located in the lower layers, however, particularly in the basal epidermis (stratum basale), actively divide and ultimately migrate up through the more superficial layers to replace those cells being sloughed off at the external surface. Cultures of human keratinocytes are used in examinations of skin structure and disease, and as in vitro models of human skin in toxicology studies (Boyce and Ham (1985) In: In Vitro Models for Cancer Research, vol. III, Webber et al., eds., Boca Raton, Fla.: CRC Press, Inc., pp. 245-274). Successful culture of keratinocytes has proven, however, to be somewhat difficult, owing primarily to their nutritional fastidiousness (Gilchrest et al., J. Cell. Physiol. 120: 377-383 (1984)). For example, in most early studies using traditional serum-supplemented culture media, keratinocytes from skin explants were rapidly overgrown by less fastidious and faster-growing fibroblasts that were also resident in the tissue (Freshney, Id.). Thus, there has been substantial work expended in the attempt to formulate culture media favoring the selection and successful in vitro cultivation of human keratinocytes. Several forms of specialized media have been developed, and are available, for the culture of keratinocytes.

A variety of systems have been developed to culture human keratinocytes. Early work in this area used specialized culture media such as Medium 199 (Marcelo et al. (1978) J. Cell Biol. 79: 356) and NCTC 168 (Price et al. (1980) In Vitro 16:147) supplemented with serum. Alternatively, keratinocyte growth and colony formation have been shown to be improved by plating cells on lethally irradiated 3T3 fibroblasts and by adding epidermal growth factor (EGF) and hydrocortisone to the medium (Rheinwald and Green (1975) Cell 6:331). One of the first serum-free medium formulations developed for keratinocyte culture was based on Medium 199 and included a growth factor cocktail comprising bovine brain extract (Gilchrest et al. (1982) J. Cell. Physiol. 112:197), and serum-free culture of human keratinocytes without the use of 3T3 fibroblast feeder layers became widely accepted upon the development of a more specialized basal medium, MCDB-153 (Boyce and Ham (1983) J. Invest. Dermatol. 81:33; and U.S. Pat. Nos. 4,673,649 and 4,940,666). Serum-free MCDB-153 includes trace elements, ethanolamine, phosphoethanolamine, hydrocortisone, EGF, and bovine pituitary extract (BPE). This medium and several enhanced versions have been used widely for human keratinocyte cultivation (Pittelkow and Scott (1986) Mayo Clin. Proc. 61: 771; Pirisi et al. (1987) J. Virol. 61:1061; Shipley and Pittelkow (1987) Arch. Dermatol. 123:1541; Daley et al. (1990) FOCUS (GIBCO/LTI) 12:68).

The use of BPE is also common to many commercially available media for keratinocyte cultivation, including KGM (Clonetics Corporation; San Diego, Calif.), CS-2.0 Keratinocyte Cell Growth Medium (Cell Systems, Inc.; Kirkland, Wash.), M154 (Cascade Biologicals, Inc.; Portland, Oreg.) and Keratinocyte-SFM (GIBCO/LTI; Gaithersburg, Md. (Catalog No. 17005). In addition, a Keratinocyte-SFM formulated without calcium chloride, to allow specialized growth in low calcium, is available (GIBCO/LTI; Gaithersburg, Md. (Catalog No. 37010).

There has also been reported a fully defined medium for the culture of epidermal cells, wherein BPE is replaced with epidermal growth factor (EGF), insulin-like growth factor 1 (IGF-1) and increased quantities of six specific amino acids (U.S. Pat. No. 5,292,655).

Other growth factors which may be optionally employed include, but are not limited to, interleukins, such as interleukin-1 and interleukin-3; fibroblast growth factors; prolactin; growth hormone; transforming growth factors such as transforming growth factor-beta; insulin-like growth factors, such as IGF-I and IGF-II; glucagon; insulin; platelet-derived growth factor; thyroid hormones, such as T3; hepatopoietins such as hepatopoietin A and hepatopoietin B; epidermal growth factors (EGF); dexamethasone; norepinephrine; and transferrin. One or more of such growth factors may be contained in a serum-free medium referred to as hormonally-defined medium, or HDM. An example of HDM is further described in Enat et al. ((1984) Proc. Nat. Acad. Sci. USA 81: 1411-1415).

The cell culture media of the present invention include aqueous-based media, comprising a number of ingredients in a solution of deionized, distilled water to form “basal media.” Ingredients that may be included in a basal medium of the present invention include are amino acids, vitamins, inorganic salts, adenine, ethanolamine, D- glucose, heparin, N-[2-hydroxyethyl]-piperazine-N′-[2-ethanesulfonic acid] (HEPES), rEGF, hydrocortisone, insulin, lipoic acid, phenol red, phosphoethanolamine, putrescine, sodium pyruvate, triiodothyronine (T3), thymidine and transferrin. Alternatively, insulin and transferrin may be replaced by ferric citrate or ferrous sulfate chelates. Each of these ingredients can be obtained commercially, for example from Sigma (Saint Louis, Mo.). The medium can also be supplemented with bovine pituitary extract and fetal bovine serum. In general, BPE and FBS are not added to medium during the initial stage of isolating liver stem cells (e.g., isolation of cells from tissue).

Amino acid ingredients that may be included in the media of the present invention include L-alanine, L-arginine, L-asparagine, L-aspartic acid, L-cysteine, L-glutamic acid, L-glutamine, glycine, L-histidine, L-isoleucine, L-leucine, L-lysine, L-methionine, L- phenylalanine, L-proline, L-serine, L-threonine, L-tryptophan, L-tyrosine, and L-valine. These amino acids can be obtained commercially, for example from Sigma (Saint Louis, Mo.).

Vitamin ingredients that can be included in a medium of the present invention include biotin, choline chloride, D-Ca⁺²-pantothenate, folic acid, myo-inositol, niacinamide, pyridoxine, riboflavin, thiamine, thioctic acid, and vitamin B₁₂. These vitamins can be obtained commercially, for example from Sigma (Saint Louis, Mo.).

Inorganic salt ingredients that can be used in a medium of the present invention include a calcium salt (e.g., CaCl₂), CuSO₄, FeSO₄, KCl, a magnesium salt (e.g., MgCl₂), a manganese salt (e.g., MnCl₂), sodium acetate, NaCl, NaHCO₃, Na₂ HPO₄, Na₂ SO₄, and ions of the trace elements selenium, silicon, molybdenum, vanadium, nickel, tin, and zinc. These trace elements may be provided in a variety of forms, generally in the form of salts such as Na₂ SeO₃, Na₂ SiO₃, (NH₄)₆Mo₇O₂₄, NH₄VO₃, NiSO₄, SnCl, and ZnSO. These inorganic salts and trace elements can be obtained commercially, for example from Sigma (Saint Louis, Mo.).

The culture media of the present invention are typically sterilized to prevent unwanted contamination. Sterilization can be accomplished, for example, by filtration through a low protein-binding membrane filter of about 0.1-0.22 μM pore size (available commercially, for example, from Millipore, Bedford, Mass.) after admixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1×sterile medium formulation. Certain concentrated solutions that are not adversely affected can be sterilized by autoclaving although generally autoclaving or other elevated temperature-based methods of sterilization are not favored, since many of the components of the present culture media are heat labile and are irreversibly degraded by temperatures such as those achieved during most heat sterilization methods.

The identification of the present media formulations (i.e., formulations suitable for isolating and propagating stem cells derived from an adult tissue) can be carried out using approaches known in the art such as those described by Ham ((1984) Methods for Preparation of Media, Supplements and Substrata for Serum-Free Animal Culture, Alan R. Liss, Inc., New York, pp. 3-21) and Waymouth ((1984) Methods for Preparation of Media, Supplements and Substrata for Serum-Free Animal Culture, Alan R. Liss, Inc., New York, pp. 23-68). The final concentrations for medium ingredients are typically identified either by empirical studies, in single component titration studies, or by interpretation of historical and current scientific literature. In single component titration studies, using animal cells, the concentration of a single medium component is varied while all other constituents and variables are kept constant and the effect of the single component on viability, growth or continued health of the animal cells is measured. For example, a medium can be identified as suitable for culturing adult stem cells if cells cultured in that medium have one or more characteristics of stem cells when cultured in the medium such as a high proliferative capacity, a stem cell-like morphology, or express one of more markers associated with a stem cell (for example, expression of Oct-4 or vimentin).

The specialized media of the invention therefore include both the modified and improved variations of known media formulations described in the examples below, and formulations within the scope of the claims and description of the invention. Accordingly, the media compositions and formulations of the invention include non- specified components described throughout the application as well as those which are known to the skilled artisan or can be otherwise deduced using routine methods.

5.4 Animal Cells

The invention provides cells, including certain stem cells of mammalian origin, for example, adult liver stem cells (e.g., human adult liver stem cells or human adult adipose stem cells). The invention provides for methods of facile propagation of such cells from dissociated adult liver tissue, thereby allowing for the isolation of adult stem cell colonies (e.g., liver stem cell colonies or adipose stem cell colonies) and, therefore, clonal adult stem cells (e.g., clonal liver stem cells or clonal adipose stem cells). The adult stem cells may be derived from a primate, and, in particular, includes human adult stem cells derived from adult (i.e., nonembryonic) human liver tissue or human adipose tissue.

The adult stem cells (e.g., liver stem cells or adipose stem cells) of the invention have, in certain instances, particular distinguishing features such as the ability to form colonies on tissue culture plastic or the ability to grow without the use of feeder cells. Generally, the adult stem cells (e.g., liver stem cells or adipose stem cells) of the invention are characterized by having a high proliferation potential, up to about the equivalent of 6, 12, 24, or 48, or 54 doublings (cell divisions) (i.e., resulting in the multiplication of one cell into up to 2⁵⁴, or over 1.6×10¹⁶, cells). The adult liver stem cells are also characterized by the expression of certain adult liver stem cell markers, including Oct-4, α1-antitrypsin, γ-glutamyl transpeptidase, alpha-fetoprotein, Thy-1, and vimentin. Adult mesenchymal stem cells (e.g., adipocyte stem cells) are characterized by certain stem cell markers including Oct-4 and vimentin. The cells can exhibit other distinguishing features such as a relatively high level of anchorage-independent growth (AIG; e.g., at least about 50%), serpiginous morphology, low or absent levels of gap junctions, and the ability to divide symmetrically (dividing to form two serpiginous cells) or asymmetrically (e.g., dividing to form one serpiginous cell and one cuboidal or fibroblast-like cell) The adult stem cells (e.g., adult liver stem cells or adult adipose stem cells) described herein may further be immortalized using methods described below (e.g., by transformation with an immortalizing gene such as SV40 large T-antigen, hTERT, a dominant-negative form of the tumor suppressor p53 or retinoblastoma (RB) gene, adenovirus E1a, adenovirus E1b, papilloma virus E6 or papilloma virus E7). Furthermore, the adult stem cells, or immortalized derivatives thereof, may be further differentiated, as described below, so that they express one or more specific functions, e.g., hepatocyte-sepecific functions (e.g., by using a differentiation-promoting media containing higher calcium concentrations than the medium in which the adult stem cell was maintained) or using a cell type-specific differentiation agent such as a hepatocyte differentiation agent (such as hepatocyte growth factor, n-butyrate or phenobarbitol). In certain instances, the isolated adult liver stem cells are differentiated so that they form hepatocytes (as judged by cell morphology, gene expression profile or other characteristics). For example, the differentiated adult liver stem cells, in certain instances, express hepatocyte-specific function such as gap-junctional intercellular communication (GJIC), P450, glucose-6-phosphatase, catalase, or P-glycoprotein. The gap-junctional intercellular communication function or activity may be reflected in the expression of mRNA or protein from a connexin gene, such as connexin 26, connexin 32, or connexin 43. Such differentiated adult liver stem cells may also be characterized by the expression of other hepatocyte activities such as 7-ethoxycoumarin O-de-ethylase, aloxyresorufin O-de-alkylase, coumarin 7-hydroxylase, p-nitrophenol hydroxylase, testosterone hydroxylation, UDP-glucuronyltransferase, glutathione S-transferase, gamma-glutamyl transpeptidase, or glucose-6-phosphatase.

Mesenchymal stem cells derived from adult adipose tissue (adipose stem cells) can be induced to differentiate into a number of cells types including osteocytes (bone), chondrocytes (cartilage), and adipocytes (fat) using different media supplementation (e.g., see Table 1).

TABLE 1
Differentiation of mesenchymal stem cells from adipose tissues by media supplementation

| Cell | Medium/ | Chemicals for | | |
|---|---|---|---|---|
| Differentiation | serum | induction | Induction procedure | |
| Adipogenesis | D-medium* + | IDI-I and Insulin | 1. | Subculture cells into 60 mm |
| | 10% FBS | cocktail | | plates in D-medium with 10% |
| | | IBMX (3-isobutyl-1- | | FBS. |
| | | methylxanthine, | 2. | The next day, differentiation |
| | | Sigma I7018) | | induction in IDI-I medium for 2 |
| | | 500 μM | | days then in Insulin-containing |
| | | Dexamethasone | | medium for 1 day. |
| | | (Sigma D8893) | 3. | Repeat the cycle (IDI-I for 2 |
| | | 1 μM | | days, Insulin for 1 day) 3 times. |
| | | Indomethacin (Sigma | 4. | Stain lipid vacuoles by Oil Red |
| | | I18280) 1 μM | | O stain. |
| | | Insulin (Sigma | | |
| | | I1882) 10 μg/ml | | |
| Chondrogenesis | D-medium* + | TAI cocktail | 1. | Prepare 10 × 10⁶ cells/ml, and |
| | 10% FBS | TGF-β1 (Sigma | | use a micropipette to deliver 10 |
| | | T1654) 10 ng/ml | | μl to the center of each well in a |
| | | L-Ascorbate-2- | | 24-well plate. |
| | | phosphate | 2. | Incubate for two and half hours, |
| | | (Sigma A8960) | | then add 1 ml TAI-containing |
| | | 50 μM | | medium for culture. |
| | | Insulin (Sigma | 3. | Medium change every three |
| | | I882) 6.25 | | days using TAI-containing |
| | | μg/ml | | medium. |
| | | | 4. | At the end of 14 days, cells or |
| | | | | cell aggregates are rinsed twice |
| | | | | with PBS and fixed in 4% |
| | | | | paraformaldehyde for 15 |
| | | | | minutes, then stained with |
| | | | | Alcian blue for sulfated |
| | | | | proteoglycan-rich matrix. |
| Osteogenesis | D-medium* + | DAG cocktail | 1. | Subculture cells into 60 mm |
| | 10% FBS | Dexamethasone | | plates in D-medium with 10% |
| | | (Sigma: D8893) | | FBS. |
| | | 0.1 μM | 2. | The next day, differentiation |
| | | L-Ascorbate-2 | | induction in DAG-containing |
| | | phosphate (Sigma: | | medium. |
| | | A8960) 50 μM | 3. | Incubate cells for 2 weeks in |
| | | β-Glycerophosphate | | DAG-containing medium with |
| | | Disodium (Sigma: | | medium change every other day. |
| | | G9891) 10 mM | 4. | Examine the formatted ECM |
| | | | | calcification by Von Kossa |
| | | | | stain. |
| Myogenesis | D-medium * + | Hydrocortisone | 1. | Subculture cells into 60 mm |
| | 5% horse | (Sigma: H0888) | | plates in D-medium with 10% |
| | serum | 50 μM | | FBS. |
| | | | 2. | The next day, the |
| | | | | hydrocortisone-containing D- |
| | | | | medium with 5% horse serum is |
| | | | | used to grow the cells for 4-6 |
| | | | | weeks, with medium renewal |
| | | | | every 3 days. |
| | | | 3. | Examine for myosin and myo- |
| | | | | D1 gene expression by |
| | | | | immunostaining after 4-6 weeks |
| | | | | incubation. |

D-medium is a modified Eagle's MEM. (Chang, C. C. et al., Somatic Cell Genetics 7: 235-253, 1981)

Methods of inducing these cell types include induction of osteocyte differentiation by culturing in a medium composed of modified MEM with 10% FBS and supplemented with dexamethasone (0.1 μM), L-ascorbate-2-phosphate (50 μM), and α-glycerophosphate (10 mM) for about four weeks. Osteocytes can be identified by the presence of calcified extracellular matrix (ECM) using Von Kossa staining. Adipocyte induction can be accomplished by culturing mesenchymal stem cells in a medium containing modified MEM with 10% FBS and supplemented with IBMX (I) (500 μM), dexamethasone (D) (1 μM), indomethacin (1) (1 μM), and insulin (I) (10 μg/ml) for three cycles of [IDI-1-2 days, insulin-1 day] (see Table 1), and repeating the cycle three times. Successful induction of adipocytes can be determined using, e.g., Oil Red 0 staining. Chondrogenic differentiation can be achieved by culturing mesenchymal stem cells in micromass culture using a medium composed of modified MEM containing 10% FBS and supplemented with TGF-β1 (10 ng/ml), L-ascorbate-2-phosphate (50 μM), and insulin (6.25 μg/ml). Cells with characteristics of chondrocytes generally develop in about one week and can be identified, e.g., using Alcian blue (pH 1.0) staining, which detects the presence of proteoglycans. Myogenic differentiation can be induced, e.g., by culturing mesenchymal stem cells in modified MEM containing 5% horse serum and supplemented with 50 μM hydrocortisone for four to six weeks. Differentiated cells can be identified, e.g., by immunostaining with an antibody that specifically recognizes skeletal myosin. The methods of inducing differentiation that are described herein are exemplary and are not intended to be limiting. Other suitable methods of identifying specific differentiated cell types are known in the art and can be used to identify differentiated cells induced to form from adult stem cells cultured using the methods described herein.

In general, cells of the invention include cells that can be grown in a medium described herein and cells of animal origin, including but not limited to, stem cells obtained from mammals. Mammalian cells suitable for cultivation in the media include stem cells of human origin, which can be primary cells derived from a tissue sample such as liver, kidney, pancreas, lung, muscle, bone, intestinal gastric, adipose, or neuronal tissue. The cells can be normal cells, or may optionally be diseased or genetically altered. Other mammalian cells, such as primate and rodent cells and derivatives thereof, can also be cultivated in the present media. Tissues, organs, and organ systems derived from animals or constructed in vitro or in vivo using methods known in the art can similarly be cultivated in a culture medium of the present invention.

Isolated liver stem cells according to the invention can be obtained from adult liver tissue, including post-embryonic, e.g., pediatric liver tissue. The cells can be obtained from adult liver tissue rather than embryonic tissue. The cells may differentiate into mature functional hepatocytes or mature bile duct cells. In most applications, liver stem cells differentiate into mature functional hepatocytes, i.e., hepatocytes characterized by liver-specific differentiated metabolic functions, e.g., the expression of albumin, CCAM, glucose-6-phosphatase, α₁-antitrypsin, or P450 enzyme activity.

Isolated mesenchymal stem cells can be obtained from any source, including fetal (nonembryonic), or adult tissue (e.g., adipose tissue) using methods known in the art, including, but not limited to, lipoaspiration. Mesenchymal stem cells (e.g., derived from adipose tissue) can develop into, e.g., chondrocytes, adipocytes, osteocytes, muscle cells (skeletal muscle or cardiomyocytes, neuronal or hematopoietic cells.

Mammalian organ donors can provide liver or adipose tissue from which stem cells are isolated. For example, tissue is obtained from a rodent, such as a mouse or rat, a dog, a baboon, a pig, or another human. The tissue may be obtained from a deceased donor, an aborted fetus, or from a living donor, e.g., from a needle biopsy, a small wedge biopsy, lipoaspiration, or a partial hepatectomy. In some cases, autologous cells are obtained from a patient, manipulated in vitro, e.g., to introduce heterologous DNA, and returned to the patient. In other cases, the cells are obtained from a heterologous donor. If the donor cells are heterologous, then donor-recipient histocompatibility can be determined. For example, class I and class II histocompatibility antigens are determined and individuals closely matched immunologically to the patient are generally selected as donors. All donors are generally screened for the presence of certain transmissible viruses (e.g., human immunodeficiency virus, cytomegalovirus, hepatitis A/B). Suitable donors are those that are free from the tested infectious diseases or do not carry the tested virus.

Tissue is generally handled using standard sterile technique and a laminar flow safety cabinet. In the use and processing of all human tissue, the recommendations of the U.S. Department of Health and Human Services/Centers for Disease Control and Prevention should be followed (Biosafety in Microbiological and Biomedical Laboratories, Richmond, J. Y. et al., Eds., U.S. Government Printing Office, Washington, D.C. 3rd Edition (1993)). The tissue collected in medium with antibiotics and antimycotic and transported in ice is cut into small pieces (e.g., 0.1×0.1 mm) using sterile surgical instruments, and then treated with an enzymatic solution (e.g., collagenase available commercially, for example, from GIBCO/LTI, Gaithersburg, Md.) to promote dissociation of cells from the tissue matrix. Lipoaspirates may not require the initial mincing step. The mixture of dissociated cells and matrix molecules are washed twice with a suitable tissue culture medium or physiological saline (e.g., Dulbecco's Phosphate Buffered Saline without calcium and magnesium). Between washes, the cells are centrifuged (e.g., at 200×g) and then resuspended in serum-free tissue culture medium. Aliquots are counted, e.g., using an electronic cell counter (such as a Coulter Counter) or are counted manually using a hemocytometer.

Tissue (e.g., liver or adipose) is enzymatically digested to dissociate cells from connective tissue while preserving the integrity of stem cells present. In vivo, the liver stem cells are likely to reside in a unique niche of the liver, i.e., the canals of Hering, and stem cells derived from this region are isolated and identified by the selective cell culture methods described herein.

Certain animal cells for culturing according to the present invention may also be obtained commercially, for example from ATCC (Rockville, Md.), Cell Systems, Inc. (Kirkland, Wash.), Clonetics Corporation (San Diego, Calif.), BioWhittaker (Walkersville, Md.), or Cascade Biologicals (Portland, Oreg.). Alternatively, cells may be isolated directly from samples of animal tissue obtained via biopsy, autopsy, donation, or other surgical or medical procedure.

Isolated cells are plated according to the experimental conditions determined by the practitioner. The examples below demonstrate at least one non-limiting, functional set of culture conditions useful for cultivation of human adult liver stem cells and a set of culture conditions useful for cultivation of human adult adipose stem cells. It is to be understood, however, that the optimal plating and culture conditions for a given animal stem cell type can easily be determined by one of ordinary skill in the art using only routine experimentation. For routine culture conditions, using the present invention, cells can be plated onto the surface of culture vessels without attachment factors. Alternatively, the vessels can be precoated with natural, recombinant, or synthetic attachment factors or peptide fragments (e.g., collagen or fibronectin, or natural or synthetic fragments thereof). Isolated cells can also be seeded into or onto a natural or synthetic three-dimensional support matrix such as a preformed collagen gel or a synthetic biopolymeric material. Use of attachment factors or a support matrix with the medium of the present invention will enhance cultivation of many attachment-dependent cells in the absence of serum supplementation.

The cell seeding densities for each experimental condition can be selected for the specific culture conditions being used. For routine culture in plastic culture vessels, an initial seeding density of, for example, 1-5×10⁴ cells per cm² is useful. In certain cases, micromass cultures are used.

Mammalian cells are typically cultivated in a cell incubator at about 37° C. The incubator atmosphere is humidified and contains about 3-10% carbon dioxide in air, although cultivation of certain cell lines may require as much as 20% carbon dioxide in air for optimal results. Culture medium pH is in the range of about 7.1-7.6, about 7.1-7.4, or about 7.1-7.3.

Cells in closed or batch culture should undergo complete medium exchange (i.e., replacing spent media with fresh media) about every 2-3 days, or more or less frequently as required by the specific cell type. Cells in perfusion culture (e.g., in bioreactors or fermenters) will receive fresh media on a continuously recirculating basis.

An adult stem cell as described herein may, optionally, be genetically altered by the introduction of heterologous DNA. A genetically-altered stem cell is one into which has been introduced, by means of recombinant DNA techniques, a nucleic acid encoding a polypeptide. The DNA is separated from the 5′ and 3′ coding sequences with which it is immediately contiguous in the naturally occurring genome of an organism, e.g., the DNA may be a cDNA or fragment thereof. The introduced DNA may supply or supplant a missing or deficient function of the host. In some cases, the underlying defect of a pathological state is a mutation in DNA encoding a protein such as a metabolic protein. In certain instances, the polypeptide encoded by the heterologous DNA lacks a mutation associated with a pathological state. In other cases, a pathological state is associated with a decrease in expression of a protein. A genetically altered stem cell may contain DNA encoding such a protein under the control of a promoter that directs strong expression of the recombinant protein. Such cells, when transplanted into an individual suffering from abnormally low expression of the protein, produce high levels of the protein to confer a therapeutic benefit. For example, the stem cell contains heterologous DNA encoding a metabolic protein such as ornithine transcarbamylase, arginosuccinate synthetase, glutamine synthetase, glycogen synthetase, glucose-6-phosphatase, succinate dehydrogenase, glucokinase, pyruvate kinase, acetyl CoA carboxylase, fatty acid synthetase, alanine aminotransferase, glutamate dehydrogenase, ferritin, low density lipoprotein (LDL) receptor, P450 enzymes, or alcohol dehydrogenase. Alternatively, the cell may contain DNA encoding a secreted plasma protein such as albumin, transferrin, complement component C3, α₂-macroglobulin, fibrinogen, Factor XIII:C, Factor IX, or α₁-antitrypsin.

One of skill in the art will recognize proteins that are advantageous to express and produce a heterologous protein in other cell types, and can, using methods known in the art, engineer a cell to express the heterologous protein.

5.5 Cell Immortalization

Adult stem cells, for example, clonal adult stem cells (e.g., liver stem cells or mesenchymal stem cells such as adipocyte stem cells), and/or derivatives thereof (e.g., hepatocytes, adipocytes, osteocytes, myoblasts, or chrondrocytes) can be immortalized by, for example, transformation with an immortalizing gene or construct.

A useful cell proliferation factor gene or immortalizing gene construct employed in the present invention includes genes derived from normal cells. These include, but are not limited to, cell proliferation factor genes. Cell proliferation factor genes essentially relates to cell proliferation and signal transduction in the normal cell, and includes those genes which function as growth factors, those which have tyrosine kinase activity in the cell membrane, those which bind to GTP in the interior of the cell membrane, those which have serine/threonine kinase activity in the cytoplasm, and those which have the ability to bind to DNA in the nucleus. As such, cell proliferation factor genes such as a ras gene, myc gene, and an hTERT gene or the like can be employed. The hTERT gene may be advantageous to employ in certain instances, because the expression of the hTERT gene is naturally enhanced in stem and progenitor cells of organs repeating regeneration over lifetime such as liver, blood, skin, intestinal mucosa, endometrium and the like.

In general, cells can be immortalized by genetically altering them by transfection or transduction with a suitable vector, homologous recombination, or other appropriate technique, so that they express an immortalizing activity (e.g., the telomerase catalytic component (TERT)). Telomerized cells are of particular interest in applications of this invention where it is advantageous to have cells that can proliferate and maintain their karyotype, for example, in pharmaceutical screening, and in therapeutic protocols where differentiated cells are administered to an individual to augment liver function, muscle function, cartilage repair, or adipose tissue function. The catalytic component of human telomerase (hTERT) is useful for this aspect, e.g., as provided in International Patent Application WO 98/14592. For certain applications, species homologs like mouse TERT (WO 99/27113) can also be used. Transfection and expression of telomerase in human cells is described in Bodnar et al. ((1998) Science 279: 349) and Jiang et al. (1999) Nat. Genet. 21:111). In another example, hTERT clones (WO 98/14592) are used as a source of hTERT encoding sequence, and spliced into an EcoRI site of a PBBS212 vector under control of the MPSV promoter, or into the EcoRI site of commercially available pBABE retrovirus vector, under control of the LTR promoter. Differentiated or undifferentiated stem cells are genetically altered using vector containing supernatants over an 8-16 hour period, and then exchanged into growth medium for 1-2 days. Genetically altered cells are selected using 100 μg/ml hygromycin B or 0.5-2.5 μg/1 nL puromycin, and recultured. They can then be assessed for hTERT expression by RT-PCR, telomerase activity (TRAP assay), immunocytochemical staining for hTERT, or replicative capacity. Continuously replicating colonies are enriched by further culturing under conditions that support proliferation, and cells with desirable phenotypes can optionally be cloned by limiting dilution.

In certain embodiments of the invention, stem cells are differentiated into cells bearing characteristics of a differentiated cell type (e.g., hepatocyte lineage, adipocyte lineage, osteocyte, lineage, chondrocyte lineage, or myoblast lineage), and then the differentiated cells are genetically altered to express TERT. In other embodiments of this invention, stem cells are genetically altered to express TERT, and then differentiated into cells bearing characteristics of the differentiated cell type (e.g., hepatocyte lineage, adipocyte lineage, osteocyte, lineage, chondrocyte lineage, or myoblast lineage). Successful modification to increase TERT expression can be determined by TRAP assay, or by determining whether the replicative capacity of the cells has improved.

Further non-limiting examples of useful immortalizing genes include: (1) nuclear oncogenes such as v-myc, N-myc, T antigen and Ewing's sarcoma oncogene (see Fredericksen et al. (1988) Neuron 1: 439-448; Bartlett et al. (1988) Proc. Natl. Acad. Sci. USA 85:3255-3259; and Snyder et al. (1992) Cell 68: 33-51); (2) cytoplasmic oncogenes such as bcr-abl and neurofibromin (Solomon et al. (1991) Science 254:1153-1160); (3) membrane oncogenes such as neu and ret (Aaronson (1991) Science 254:11531161), (4) tumor suppressor genes such as mutant p53 and mutant Rb (retinoblastoma) (Weinberg (1991) Science 254: 1138-1146), and (5) other immortalizing genes such as Notch dominant negative (Coffman et al. (1993) Cell 23:659-671). Useful oncogenes for the purpose of immortalization include v-myc and the SV40 T antigen.

The foreign (heterologous) immortalizing nucleic acid can be introduced or transfected into a multipotent adult cell (e.g., a liver cell or adipocyte cell) or its progeny. A multipotent adult stem cell or its progeny that harbors such foreign DNA may be said to be a genetically engineered stem cell or stem cell derivative. The foreign DNA may be introduced using a variety of techniques.

In certain instances, the foreign DNA is introduced into a multipotent stem cell using the technique of retroviral transfection. Recombinant retroviruses harboring the gene(s) of interest are used to introduce marker genes, such as the E. coli beta- galactosidase (lacZ) gene, or oncogenes. The recombinant retroviruses are produced in packaging cell lines to produce culture supernatants having a high titer of virus particles (generally 10⁵ to 10⁶ pfu/ml). The recombinant viral particles are used to infect cultures of the stem cells (e.g., adult liver stem cells or adult adipose stem cells) or their progeny by, for example, incubating the cell cultures with medium containing the viral particles and 8 μg/ml polybrene for three hours. Following retroviral infection, the cells are rinsed and cultured in standard medium. The infected cells are then analyzed for the uptake and expression of the foreign DNA. The cells can be subjected to selective conditions that select for cells that have taken up and expressed a selectable marker gene.

A retroviral vector may be used for transferring the cell proliferation factor gene into an adult stem cell (e.g., liver stem cell or adipocyte stem cell) or stem cell derivative (e.g., a differentiated hepatocyte, adipocyte, myoblast, osteocyte, or chondrocyte). The retroviral vector may be used as means for transferring a foreign gene into the animal stem cells, or derivatives thereof, of the invention. Since the gene transferred by the retroviral vector is integrated into chromosomal DNA of the host stem cell, the gene is absolutely transmitted to the daughter cell and therefore can be expressed stably over long period.

Any process can be used to transfer the retroviral vectors into the culture cells. For example, the transferring can be performed by culturing cells that produce the retroviral vectors, and then inoculating the resulting culture supernatant on the adult stem cell to be transformed with the immortalizing gene. Various conditions such as culture condition and seeding density about each kind of cell can be easily determined according to the process well known in the art.

In other instances, the foreign DNA is introduced using the technique of calcium- phosphate-mediated transfection. A calcium-phosphate precipitate containing DNA encoding the gene(s) of interest is prepared using the technique of Wigler et al. ((1979) Proc. Natl. Acad. Sci. USA 76:1373-1376). Cultures of the adult stem cells (e.g., liver stem cells or adipose stem cells) or their progeny are established in tissue culture dishes. Twenty-four hours after plating the cells, the calcium phosphate precipitate containing approximately 20 μg/ml of the foreign DNA is added. The cells are incubated at room temperature for 20 minutes. Tissue culture medium containing 30 μM chloroquine is added and the cells are incubated overnight at 37° C. Following transfection, the cells are analyzed for the uptake and expression of the foreign DNA. The cells may be subjected to selection conditions that select for cells that have taken up and expressed a selectable marker gene.

The immortalizing factor gene used in the present invention can be inserted between a pair of site-specific recombination sequences so that the gene can be excised later from the pro-virus transferred into an adult stem cell, or derivative thereof. “Site- specific recombinant sequence” is a specific base sequence recognized by a site-specific recombinase. In between the specific sequences, homologous recombination comprising the steps of a DNA-strand excision, an exchange of the strands and a coupling thereof are performed. Representative site-specific recombinant sequences include the LoxP sequence, the FRT sequence, or the like. The LoxP sequence is a sequence comprising 34 bases of ATAACTTCGTATAGCATACATTATACG- AAGTTAT (SEQ. ID NO: 1) for performing homologous recombination by Cre recombinase alone. When a pair of LoxP sequences inserted in the same direction presents in a same DNA molecule, the DNA sequence inserted between them is excised to become a circular molecule (excision reaction).

Further, in the present invention, it may be useful in certain instances to insert a selection marker, such as a green fluorescent protein (GFP) gene, between the pair of site-specific recombinant sequences whenever the cell proliferation factor gene is transferred into the target cell. The GFP gene is thus useful to selectively identify the immortalized stem cell, or derivative thereof, which is infected with the retroviral vector and wherein a pro-virus is integrated into the genome, by using FACS (fluorescence activated cell sorter). Alternatively, a drug-resistance gene may be used instead of the GFP gene as long as the stem cell wherein the pro-virus is integrated into genome is identified selectively.

Examples of drug-resistance genes for use in the invention include hygromycin resistance gene, neomycin resistant gene, ampicillin resistance gene, E. coli gpt gene or the like. The invention is not limited by a specific drug-resistance marker.

Methods in molecular genetics and genetic engineering are described in, for example, Molecular Cloning: A Laboratory Manual, 2nd Ed. (Sambrook et al., Cold Spring Harbor Press, 1989); Oligonucleotide Synthesis (Gait, ed., 1984); Animal Cell Culture (Freshney, ed., 1987); the series Methods in Enzymology (Academic Press, Inc.); Gene Transfer Vectors for Mammalian Cells (I. M. Miller & M. P. Calos, eds., 1987); Current Protocols in Molecular Biology and Short Protocols in Molecular Biology, 3rd Edition (Ausubel et al., eds., 1987 & 1995); and Recombinant DNA Methodology II (Wu ed., Academic Press, 1995). Reagents, cloning vectors, and kits for genetic manipulation referred to in this disclosure are available from commercial vendors such as, for example, BioRad, Stratagene, Invitrogen, ClonTech, and Sigma Chemical Co.

General techniques used in raising, purifying and modifying antibodies, and the design and execution of immunoassays including immunohistochemistry, the reader is referred to Handbook of Experimental Immunology (Weir and Blackwell, eds.); Current Protocols in Immunology (Coligan et al., eds., (1991); and Masseyeff, Albert, and Staines, eds., Methods of Immunological Analysis (Weinheim: VCH Verlags GmbH, 1993).

An immortalized stem cell derived by the methods described herein or other methods known in the art, most usefully, cells that are not tumorigenic, optionally have a shape like a normal stem cell derived from the tissue of origin (e.g., liver or adipose tissue), optionally retain pluripotency of adult stem cells (e.g., stem cells derived from liver or adipose tissue), and are able to grow, at least in a short term, using the culture media and conditions described herein.

Further cultivation of the immortalized stem cells described herein can be carried out under a condition where cell-growth rate is controlled. A doubling time of an immortalized stem cell (e.g., a liver stem cell or an adipose stem cell) can be from 24 to 72 hours, for example, from 24 to 48 hours, or from 24 to 36 hours. The culture medium for the immortalized stem cells can be a culture medium described herein or a culture medium known in the art.

5.6 Cell Differentiation and Trans-differentiation

In another aspect of the invention, adult stem cells (e.g., liver stem cells or adipose stem cells) are used as a source of differentiated cells, for example, hepatocytes, adipocytes, osteocytes, chondrocytes, or myoblasts that are useful for replacing or supplanting cells damaged in the course of disease, infection, or from congenital abnormalities. These methods of stem cell differentiation (e.g., into hepatocytic, adipocyte, osteocyte, chondrocyte, or myoblast cell types) are known in the art and certain methods are further described below.

Also included are methods of adult stem cell, including adult liver stem cell and adult adipose cell, trans-differentiation. Trans-differentiation allows the differentiation of stem cell derived from one type of tissue, for example liver stem cells derived from adult liver tissue, into differentiated cells of a different type of tissue, for example pancreatic tissue. Other non-limiting examples of trans-differentiation include the differentiation of adipocyte stem cells derived from adult adipose tissue into osteocytes, chondrocytes, or myoblasts. Exemplary methods of stem cell trans-differentiation are also described further below.

The differentiated cells can be generated by culturing the stem cells in a growth environment that includes a differentiation agent (e.g., a hepatocyte differentiation agent, adipocyte differentiation agent, osteocyte differentiation agent, chondrocyte differentiation agent, or myoblast differentiation agent, such as a calcium ion concentration higher than that used to propagate the adult stem cells (e.g., higher than that of an adult stem cell medium described herein, or greater than about 0.2 mM Ca⁺²) or n-butyric acid or other differentiation agent. As termed herein, a differentiation can include one or more compounds.

For example, the histone deacetylase inhibitors, including butyrate and trichostatin A, have been implicated in the differentiation of a wide variety of cell types, and butyrate may be used to drive certain pluripotent stem cell populations to differentiate into remarkably homogeneous populations of hepatocytes (see U.S. Pat. No. 6,506,574). Furthermore, butyrate has been shown to have a differentiating and modulating effect on a variety of other cell types, both in culture and in vivo. Indeed, Kosugi et al. ((1999) Leukemia 13: 1316) and Tamagawa ((1998) Biosci. Biotechnol. Biochem. 62:1483) reported that histone deacetylase inhibitors are potent inducers of differentiation in acute myeloid leukemia cells. Davis et al. ((2000) Biochem J. 346 pt 2: 455) and Rivero et al. ((1998) Biochem. Biophys. Res. Commun. 248: 664) discuss the effect of butyrate in erythroblastic differentiation. Perrine et al. ((1994) Am. J. Pediatr. Hematol. Oncol. 16:67), and Perrine et al. ((1993) N. Engl. J. Med. 328:81) have proposed butyrate derivatives as agents for stimulating fetal globin production in beta- globin disorders.

The differentiation agent can be added directly to undifferentiated stem cells cultured with or without feeder cells. Alternatively, the adult liver stem cells are allowed to differentiate into a mixed cell population, and the differentiation agent is added to the mixed population. This results in a less heterogeneous population, in which a substantial proportion of the cells have the desired phenotype. In some instances, the culture method for hepatocyte differentiation also includes hepatocyte maturation factors such as solvents like DMSO, growth factors like FGF, EGF, and hepatocyte growth factor, and glucocorticoids like dexamethazone. For differentiation into other cell types, e.g., by adipocyte stem cells to adipocytes, osteocytes, chondrocytes, or myoblasts, other differentiation agents can be used including those known in the art and those described infra (e.g., Zuk et al. (2001) Tissue Engineering 7: 211; Zuk et al. (2002) Mol. Biol. of the Cell 13: 4279; Pittenger et al. (1999) Science 284:143).

The manipulation and growth of certain early liver progenitor cells from embryonal and neonatal rat livers has been described (see, e.g., Agelli et al. (1997) Histochem. J. 29: 205; Brill et al. (1999) Dig. Dis. Sci. 44: 364) and conditions for expansion have been described (see U.S. Pat. No. 5,576,207). Furthermore, Michalopoulos, et al. ((1999) Hepatol. 29: 90) report a system for culturing rat hepatocytes and nonparenchymal cells in biological matrices, and Block et al. ((1996) J. Cell Biol. 132: 1133) developed conditions for expansion, clonal growth, and specific differentiation in primary cultures of hepatocytes induced by a combination of growth factors in a chemically defined medium. It has believed that mature rat liver cells derive from certain precursor cells (sometimes referred to as “hepatoblasts” or “oval cells”) that have the capacity to differentiate into either mature hepatocytes or biliary epithelial cells (see Rogler (1997) Am. J. Pathol. 150: 591; Alison (1998) Current Opin. Cell Biol. 10: 710; Lazaro, et al. (1998) Cancer Res. 58: 514; Germain, et al., (1988) Cancer Res. 48: 4909). Furthermore, European Patent Application EP 953 633 A1 proposes a cell culturing method and medium for producing proliferated and differentiated human liver cells from donated human liver tissue.

These methods of obtaining differentiated hepatocytes can be supplemented, if desired, by the use of separate compounds or mixtures of compounds that act as hepatocyte maturation factors. Such agents may augment the phenotype change promoted by the differentiation agent, or they may push the differentiation pathway further towards more mature cells, or they may help select for cells of the hepatocyte lineage (for example, by preferentially supporting their survival), or they may promote more rapid proliferation of cells with the desired phenotype.

Once class of hepatocyte maturation factors are soluble growth factors (peptide hormones, cytokines, ligand-receptor complexes, and the like) that are capable of promoting the growth of cells of the hepatocyte lineage. Such factors include, but are not limited to, epidermal growth factor (EGF), insulin, TGF-alpha, TGF-beta, fibroblast growth factor (FGF), heparin, hepatocyte growth factor (HGF), Oncostatin M in the presence of dexamethazone, IL-1, IL-6, IGF-I, IGF-II, HBGF-1, and glucagon.

Another class of hepatocyte maturation factors is corticosteroids, for example, glucocorticoids. Such compounds are a steroid or steroid mimetic, and affects intermediary metabolism, especially promotion of hepatic glycogen deposition, and inhibiting inflammation. Included are naturally occurring hormones exemplified by cortisol, and synthetic glucocorticoids such as dexamethazone (U.S. Pat. No. 3,007,923) and its derivatives, prednisone, methylprednisone, hydrocortisone, and triamcinolone (U.S. Pat. No. 2,789,118) and its derivatives.

Still another class of hepatocyte maturation factors are certain organic solvents, like DMSO. Alternatives with similar properties include but are not limited to dimethylacetamide (DMA), hexmethylene bisacetamide, and other polymethylene bisacetamides. Solvents in this class are related, in part, by the property of increasing membrane permeability of cells.

Testing for whether a candidate compound acts as a maturation factor (e.g., hepatocyte maturation factor) for the purpose of this invention is performed empirically: adult stem cell cultures (e.g., adult liver stem cell cultures) are differentiated into cells of the desired lineage (e.g., hepatocyte lineage) using a differentiation agent (e.g., hepatocyte differentiation agent) described above, in combination with a model maturation factor (e.g., a hepatocyte maturation factor), such as a growth factor or DMSO (the positive control). In parallel, adult stem cells (e.g., adult liver stem cells) are subjected to a similar protocol using the same differentiation agent and a candidate maturation factor. Resultant cells are then compared phenotypically to determine whether the maturation factor has a similar effect to that of the positive control.

In particular applications of this aspect of the invention, the differentiation agent and the maturation factor may be used simultaneously or sequentially. In one illustration, newly plated adult liver stem cell cultures are placed in a medium containing both n- butyrate and DMSO, and cultured for 4, 6, or 8 days, or until characteristic features appear, replacing the medium periodically with fresh medium containing n-butyrate and DMSO. In another illustration, adult liver stem cell cultures are first cultured with n- butyrate and DMSO for 4, 6, or 8 days, then the medium is exchanged for a hepatocyte- friendly medium containing a cocktail of growth factors (optionally, in combination with n-butyrate) for long-term culture or assay.

Mesenchymal stem cells (e.g., adipose stem cells) can be induced to differentiate into adipocytes, osteocytes, chondrocytes, myocytes, or neuronal cells (e.g., Zuk et al. (2001) Tissue Engineering 7: 211; Zuk et al. (2002) Mol. Biol. of the Cell 13: 4279; Pittenger et al. (1999) Science 284:143; Mizuno et al. (2002) Plast. Reconstr. Surg. 109: 199; Blanat-Benard et al. (2004) Circ. Res. 94:223; Rangapa et al. (2003) Ann. Thorac. Surg. 75:775).

Following these guidelines, the ability of particular compound or combination of compounds to act as maturation factors can be assessed. The effect of the compound on cell morphology, marker expression, enzymatic activity, proliferative capacity, or other features of interest is then determined in comparison with parallel cultures that did not include the candidate compound. For optimum results, several concentrations of the test compound can be evaluated. A suitable base concentration for organic solvents may be isoosmolar or isotonic with effective DMSO concentrations. Suitable base concentrations for growth factors, cytokines, and other hormones may be concentrations known to have similar growth-inducing or hormone activity in other systems. The test compound can then be tested over a range of about 1/10th to 10 times the base concentration to determine if it has the desired effect on stem cell or differentiated cell- directed maturation of the adult stem cells (e.g., adult liver stem cells or adipose stem cells).

Hepatocytes can be characterized according to a number of phenotypic criteria. The criteria include but are not limited to the detection or quantitation of expressed cell markers, enzymatic activities, and the characterization of morphological features and intercellular signaling. Characteristics of differentiated hepatocytes for use in the invention include any or all of the following: antibody-detectable expression of α₁-antitrypsin or albumin; absence of antibody-detectable expression of alpha-fetoprotein; expression of asialoglycoprotein receptor at a level detectable by reverse PCR amplification; evidence of glycogen storage; evidence of cytochrome p450 or glucose-6-phosphatase activity; expression of gap-junction intercellular communication (GJIC) activity (and/or a GJIC protein such as connexin 26 or connexin 43); as well as morphological features of hepatocytes. Certain differentiated hepatocyte cell populations of the invention have more of these hepatocyte characteristics in a greater proportion of the cells in the population. It is understood that the cells may replicate to form progeny, both during differentiation, and in subsequent manipulation. Such progeny also fall within the scope of the invention in all instances where not explicitly excluded.

For example, the differentiated hepatocytes of the invention have morphological features which may be readily appreciated by those skilled in the art, and may include any or all of the following: a polygonal cell shape, a binucleate phenotype, the presence of rough endoplasmic reticulum for synthesis of secreted protein, the presence of Golgi- endoplasmic reticulum lysosome complex for intracellular protein sorting, the presence of peroxisomes and glycogen granules, relatively abundant mitochondria, and the ability to form tight intercellular junctions resulting in creation of bile canalicular spaces. A number of these features present in a single cell is consistent with the cell being a member of the hepatocyte lineage. Unbiased determination of whether cells have morphologic features characteristic of hepatocytes can be made by coding micrographs of differentiated hepatocytes, and one or more negative control cells, such as a fibroblast, and then evaluating the micrographs in a blinded fashion.

The differentiated hepatocytes of the invention can also be characterized according to whether they express phenotypic markers characteristic of cells of the hepatocyte lineage. Cell markers useful in distinguishing liver progenitors, hepatocytes, and biliary epithelium include: albumin, α₁-antitrypsin, alpha-fetoprotein, CEA, γ-glutamyl transpeptidase, GST-P, glucose-6-phosphatase, catalase, M2-PK, L-PK, P450 mono-oxygenase, P-glycoprotein, CK8, CK18, HBD. 1, H.2, H.4, -4, H-6, HES6, RL16/79, RL23/36, HepParl, Cell-CAM, and DPP IV (see p. 35 of Sell and Zoran (1997) Liver Stem Cells, R. G. Landes Co., TX; and p 242 of Grisham et al. (1997) Stem Cells Academic Press).

It has been reported that hepatocyte differentiation requires the transcription factor HNF4 α (Li et al. (2000) Genes Dev. 14: 464). Markers independent of HNF-4 α expression include α₁-antitrypsin, alpha-fetoprotein, apoE, glucokinase, insulin-like growth factors 1 and 2, IGF-1 receptor, insulin receptor, and leptin. Markers dependent on HNF-4 α expression include albumin, apoAI, apoAII, apoB, apoCIII, apoCII, aldolase B, phenylalanine hydroxylase, L-type fatty acid binding protein, transferrin, retinol binding protein, and erythropoietin (EPO). Still other markers of interest are discussed further herein.

Assessment of the level of expression of such markers can be determined in comparison with other cells. Positive controls for the markers of mature hepatocytes include adult hepatocytes of the species of interest, and established hepatocyte cell lines, such as the HepG2 line derived from a hepatoblastoma reported in U.S. Pat. No. 5,290,684. However, permanent cell lines such as HepG2 may be metabolically altered, and fail to express certain characteristics of primary hepatocytes such as cytochrome p450. Cultures of primary hepatocytes may also show decreased expression of some markers after prolonged culture. Negative controls include cells of a separate lineage, such as an adult fibroblast cell line, or retinal pigment epithelial (RPE) cells. Undifferentiated adult liver stem cells may be positive for some of the markers listed above, but negative for markers of mature hepatocytes.

Markers for mesenchymal stem cells and their differentiated cell types are known in the art, examples of which are included herein (e.g., Silva et al. (2003) Stem Cells 21:661; De Ugarte et al. (2003) Immunology letters 89:267; NIH Report on Stem Cells: Scientific progress and future research directions (2001) Appendix E.ii.).

Tissue-specific protein and oligosaccharide determinants listed in this disclosure can be detected using any suitable immunological technique such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. Expression of an antigen by a cell is said to be “antibody-detectable” if a significantly detectable amount of antibody will bind to the antigen in a standard immunocytochemistry or flow cytometry assay, optionally after fixation of the cells, and optionally using a labeled secondary antibody or other conjugate (such as a biotin-avidin conjugate) to amplify labeling.

The expression of tissue-specific markers can also be detected at the mRNA level by Northern blot analysis, dot-blot hybridization analysis, or by reverse transcriptase initiated polymerase chain reaction (RT-PCR) using sequence-specific primers in standard amplification methods. See U.S. Pat. No. 5,843,780 for further details. Sequence data for the particular markers listed in this disclosure can be obtained from public databases such as GenBank (URL www.ncbi.nIm.nih.gov:80/entrez). Expression at the mRNA level is said to be “detectable” according to one of the assays described in this disclosure if the performance of the assay on cell samples according to standard procedures in a typical controlled experiment results in clearly discernable hybridization or amplification product. Expression of tissue-specific markers as detected at the protein or mRNA level is considered positive if the level is at least 2-fold, and, in certain instances, more than 10- or 50-fold above that of a control cell, such as an undifferentiated adult liver stem cell, a fibroblast, or other unrelated cell type.

Differentiated hepatocytes can also be characterized according to whether they display enzymatic activity that is characteristic of cells of the hepatocyte lineage. For example, assays for glucose-6-phosphatase activity are described by Bublitz ((1991) Mol Cell Biochem. 108:141); Yasmineh et al. ((1992) Clin. Biochem. 25:109); and Ockerman ((1968) Clin. Chim. Acta 17:201). Assays for alkaline phosphatase (ALP) and 5-nucleotidase (5′-Nase) in liver cells are described by Shiojiri ((1981) J. Embryol. Exp. Morph. 62:139). In addition, a number of laboratories that serve the research and health care sectors provide assays for liver enzymes as a commercial service.

Cytochrome P450 is a key catalytic component of the mono-oxygenase system. It constitutes a family of hemoproteins responsible for the oxidative metabolism of xenobiotics (administered drugs), and many endogenous compounds. Different cytochromes present characteristic and overlapping substrate specificity. Most of the biotransforming ability is attributable by the cytochromes designated 1A2, 2A6, 2B6, 3A4, 2C9-11, 2D6, and 2E1 (see pp 129-153 of Gomes-Lechon et al. (1997) In vitro Methods in Pharmaceutical Research, Academic Press). A number of assays are known in the art for measuring cytochrome p450 enzyme activity. For example, cells can be contacted with a non-fluorescent substrate that is convertible to a fluorescent product by p450 activity, and then analyzed by fluorescence-activated cell counting (U.S. Pat. No. 5,869,243). Specifically, the cells are washed, and then incubated with a solution of 10 μM/L 5,6-methoxycarbonylfluorescein (Molecular Probes, Eugene Oreg.) for 15 minutes at 37° C. in the dark. The cells are then washed, trypsinized from the culture plate, and analyzed for fluorescence emission at about 520-560 nm. A cell is said to have the enzyme activity assayed for if the level of activity in a test cell is more than 2-fold, and typically more than 10- or 100-fold above that of a control cell, such as a fibroblast.

The expression of cytochrome P450 can also be measured at the protein level, for example, using specific antibody in Western blots, or at the mRNA level, using specific probes and primers in Northern blots or RT-PCR (See Borlakoglu et al.(1993) Int. J. Biochem. 25: 1659). Particular activities of the p450 system can also be measured: 7-ethoxycoumarin O-de-ethylase activity, aloxyresorufin O-de-alkylase activity, coumarin 7-hydroxylase activity, p-nitrophenol hydroxylase activity, testosterone hydroxylation, UDP-glucuronyltransferase activity, glutathione S-transferase activity, and others (see pp 411431 in Gomes-Lechon, et al. (1997) In vitro Methods in Pharmaceutical Research, Academic Press, 1997).

Once cells of the desired phenotype are obtained, the cells can be harvested for any desired use. In certain differentiated cell populations of this invention, the cells are sufficiently uniform in phenotype such that they can be harvested simply by releasing the cells from the substrate (e.g., using collagenase or by physical manipulation), and optionally washing the cells free of debris. If desired, the harvested cells can be further processed by positive selection for desired features, or negative selection for undesired features. For example, cells expressing surface markers or receptors can be positively or negatively selected by incubating the population with an antibody or conjugate ligand, and then separating out the bound cells—for example, by labeled sorting techniques, or adsorption to a solid surface. Negative selection can also be performed by incubating the population with a cytolytic antibody specific for the undesired marker, in the presence of complement.

If desired, harvested cells can be transferred into other culture environments, such as those described elsewhere for the propagation of other types of hepatocyte preparations (see, for example, U.S. Pat. Nos. 5,030,105 and 5,576,207; EP Patent Application EP 953,633; Angelli et al. (1997) Histochem. J. 29: 205; and Gomez-Lechon et al., p. 130, In In vivo Methods in Pharmaceutical Research, Academic Press, 1997).

The replication capacity of human hepatocytes and other stem cell types, including partially differentiated cells, in culture has been generally poor, yielding disappointing culture cell populations. Accordingly, in certain applications the differentiated hepatocytes of the invention are immortalized by transfecting with large T antigen of the SV40 virus (U.S. Pat. No. 5,869, 243) or any of the other immortalizing genes or associated constructs described herein.

Since the adult stem cells (e.g., adult liver stem cells and adipocyte stem cells), and immortalized derivatives thereof, described herein can essentially be grown indefinitely, the system provides a virtually unlimited supply of differentiated cells (e.g., hepatocytic cells) for use in research, pharmaceutical development, and the therapeutic management of disease (e.g., liver disease).

Further general information and methodology relating to cells of hepatocyte lineage may be found in Liver Stem Cells (Sell and ilic (1997) in Stem Cell Biology R. G. Landes Co); Reid (1990) Curr. Opinion Cell Biol. 2: 121); and in Liver Stem Cells (Grisham (1997) in Stem Cells pp 232-282, Academic Press). Use of hepatocyte-like cells in pharmaceutical research is also described in In vitro Methods in Pharmaceutical Research (Academic Press, 1997).

The invention further provides methods of stem cell trans-differentiation in which differentiated cells of one tissue type are derived from stem cells obtained from a different tissue type. For example, Yang et al. ((2002) Proc. Natl. Acad. Sci. USA 99:8078-83) and Ber et al. ((2003) J. Biol. Chem. 278:31950-57) have shown trans- differentiation of adult liver stem cells into pancreatic endocrine hormone-producing cells using two different methods. In particular, Yang et al. have effected liver stem cell differentiation into a pancreatic differentiated cell type using a “glucose challenge” (i.e. growth of the liver stem cells in a high glucose cell culture medium (23 mM glucose, by the addition of 17.5 mM glucose to medium as described by Ramiya et al. ((2000) Nat. Med. 6: 278-82). Accordingly, this method as well as other methods of trans- differentiation into other cell types using analogous environmental stimuli, are available for use in the invention. Furthermore, Ber et al. effected trans-differentiation of liver stem cells into pancreatic cells by a different method, namely using ectopic and transient expression of the pancreatic and duodenal homeobox gene (PDX-1) in liver in vivo with the recombinant adenovirus expression construct Ad-CMV-PDX-1 (see Ferber, et al. (2000) Nat. Med. 6: 568-72; and Seijffers et al. (1999) Endocrinol. 140: 3311-17). Accordingly, this method as well as other methods of trans-differentiation into other cell types using analogous master developmental regulatory genes, are available for use in the invention. Examples of other master developmental regulatory genes include the Pax gene family (review in Underhill (2000) Biochem. Cell Biol. 78: 629-38), including Pax-6, which controls eye development in vertebrates and invertebrates, and Myo D (and Pax-3) which is involved in muscle development (see Bober et al. (1994) Develop. 120: 603-12).

Still other methods for stem cell trans-differentiation are known in the art and may be used in the instant invention. For example, both bone marrow stem cells and hepatic oval cells (HOCs) have been shown to trans-differentiate into neural cells by adopting the morphology and antigenic phenotype of both macro- and microglia cells following transplantation to the brain (see Deng et al. (2003) Exp. Neurol. 182: 373-82). Furthermore, Grimaldi et al. ((1997) Protaglandins Leukot. Essent. Fatty Acids 57: 71-5) has shown the trans-differentiation of myoblasts into adipose cells. In addition, Master et al. ((2003) J. Urol. 170: 1628-32), describe the use of urothelium to effect trans- differentiation of fibroblasts into smooth muscle cells.

5.7 Methods of Treatment with Cell Systems and Bioartificial Tissue Systems

A method of transplantation (e.g., hepatic, adipocytes, osteocytes, chondrocytes, or myoblasts) is also encompassed by the invention. A patient in need of a tissue transplant, e.g., a liver transplant (such as one suffering from degenerative liver disease, cancer, or a metabolic disease), is treated by transplanting into the patient an adult stem cell (e.g., adult liver stem cell) or stem cell-derived differentiated cell (e.g., a hepatocyte) or immortalized derivative thereof. Furthermore, to treat an inherited or acquired genetic or metabolic disease, a genetically altered stem cell (singly or paired with a differentiated cell such as a hepatocyte) may be transplanted. For example, the stem cell may be transfected with DNA encoding Factor VIII: C, Factor IX, α₁ antitrypsin, or low density lipoprotein receptor useful for treating human diseases such as hemophilia A and B, α₁ antitrypsin deficiency, and familial hypercholesterolemia, respectively.

Mesenchymal stem cells can be used for cell-based therapy of, e.g., bone damage, osteoporosis, osteoarthritis, muscle loss from trauma or tumor resection, degenerative muscle disease such as muscular dystrophy, myocardial infarction, soft tissue repair, cosmetic/reconstructive surgery, and spinal cord injury. Genetically-altered stem cells are useful as an in vivo recombinant protein delivery system and have the advantage of being capable of immortality (and thus, greater long-term survival) compared to differentiated cells, i.e., stem cells are capable of giving rise to differentiated progeny but retain the capacity for self-renewal.

The cells of the invention are also useful as the biological component of a perfusion device or as a source of functional differentiated hepatocytes which can then be used as the biological component of a perfusion device such as a liver assist device (LAD) or bioreactor. Contacting a patient-derived bodily fluid with such hepatocytes results in detoxification of the bodily fluid for subsequent return to the patient. Therefore the invention also provides for the use of differentiated adult liver stem cells to restore a degree of liver function to a subject needing such therapy, perhaps due to an acute, chronic, or inherited impairment of liver function.

To demonstrate the suitability of differentiated adult stem cells (e.g., adult liver stem cells or adipocyte stem cells) for therapeutic applications, the cells can first be tested in a suitable animal model. At one level, cells are assessed for their ability to survive and maintain their phenotype in vivo. Differentiated stem cells are administered to immunodeficient animals (such as SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed as to whether stem cells are still present. This can be performed by providing the administered cells with a detectable label (such as green fluorescent protein, or beta-galactosidase); or by measuring a constitutive marker specific for the administered cells. Where differentiated stem cells are being tested in a rodent model, the presence and phenotype of the administered cells can be assessed by immunohistochemistry or ELISA using human-specific antibody, or by RT-PCR analysis using primers and hybridization conditions that cause amplification to be specific for human polynucleotide sequences. General descriptions for determining the fate of hepatocyte-like cells in animal models are known in the art (see, e.g., Grompe et al. (1999) Sem. Liver Dis. 19: 7); Peeters et al. (1997) Hepatol. 25 :884) and Ohashi et al. ((2000) Nature Med. 6: 327). Mesenchymal stem cells and their derivatives can be assessed using animal models known in the art (non-limiting examples include, rabbit or dog models of cartilage defects, rat or sheep models of bone defects, mouse or dog models for muscle defects, and rat or mouse models for soft tissue repair).

The differentiated adult stem cells (e.g., adult liver stem cells or mesenchymal stem cells (e.g., adipocyte stem cells)) may also be assessed for their ability to restore organ function in an animal lacking full function of that organ (e.g., liver). For example, Braun et al. ((2000) Nature Med. 6:320) provide a model for toxin-induced liver disease in mice transgenic for the HSV tk gene. Furthermore, Rhim et al. (1995) Proc. Natl. Acad. Sci. USA 92: 4942) and Lieber et al. (1995) Proc. Natl. Acad. Sci. USA 92: 6210) provide models for liver disease by expression of urokinase. Mignon et al. ((1998) Nature Med. 4: 1185) describes liver disease induced by antibody to the cell-surface marker Fas. Also Overturf et al. ((1998) Human Gene Ther. 9:295) have developed a model for Hereditary Tyrosinemia Type I in mice by targeted disruption of the Fah gene. The animals can be rescued from the deficiency by providing a supply of 2-(2-nitro-4-fluoro- methyl-benzyol)-1,3-cyclohexanedione (NTBC), but develop liver disease when NTBC is withdrawn.

Acute liver disease can also be modeled by 90% hepatectomy (Kobayashi et al. (2000) Science 287:1258), or by treating animals with a hepatotoxin such as galactosamine, CCl₄, or thioacetamide. Chronic liver diseases such as cirrhosis can be modeled by treating animals with a sub-lethal dose of a hepatotoxin long enough to induce fibrosis (Rudolph et al. (2000) Science 287: 1253). Assessing the ability of differentiated cells to reconstitute liver function involves administering the cells to such animals, and then determining survival over a 1 to 8 week period or more, while monitoring the animals for progress of the condition. Effects on hepatic function can be determined by evaluating markers expressed in liver tissue, cytochrome p450 activity, and blood indicators, such as alkaline phosphatase activity, bilirubin conjugation, and prothrombin time, and survival of the host any improvement in survival, disease progression, or maintenance of hepatic function according to any of these criteria relates to effectiveness of the therapy, and can lead to further optimization.

The invention further includes differentiated cells that are encapsulated, or part of a bioartificial tissue or organ device (e.g., a bioartificial liver). For example, various forms of encapsulation have been described (see, e.g., Cell Encapsulation Technology and Therapeutics, Kuhtreiber et al. eds., Birkhauser, Boston, Mass., 1999). Differentiated cells of this invention can be encapsulated according to such methods for use either in vitro or in vivo.

Bioartificial organs for clinical use are designed to support an individual with impaired organ function (e.g., liver function)—either as a part of long-term therapy, or to bridge the time between a fulminant organ (e.g., hepatic) failure and organ (e.g., hepatic) reconstitution or transplant (e.g., liver transplant). Bioartificial liver devices are reviewed by Macdonald et al., (see pp. 252-286 of Cell Encapsulation Technology and Therapeutics) as well as in U.S. Pat. Nos. 5,270,192, 5,290,684, 5,605,835, 5,624,840, 5,837,234, 5,853,717, 5,935,849, 5,981,211 and 6,294,380, the contents of each of which are incorporated herein in their entirety.

Suspension-type bioartificial livers comprise cells suspended in plate dialysers, or microencapsulated in a suitable substrate, or attached to microcarrier beads coated with extracellular matrix. Alternatively, hepatocytes can be placed on a solid support in a packed bed, in a multiplate flat bed, on a microchannel screen, or surrounding hollow fiber capillaries. The device has inlet and outlet through which the subject's blood is passed, and sometimes a separate set of ports for supplying nutrients to the cells.

Current proposals for such liver support devices involve hepatocytes from a xenogeneic source, such as a suspension of porcine hepatocytes, because of the paucity of available primary human hepatocytes. Xenogeneic tissue sources raise concerns regarding immunogenicity and possible cross-species viral transmission.

The invention provides a system for generating preparative cultures of human cells. Differentiated stem cells (e.g., adult liver stem cells or adipocyte stem cells) are prepared according to the methods described herein, and then plated into the device on a suitable substrate, such as a matrix of Matrigel™ or collagen. The efficacy of the device can be assessed by comparing the composition of blood in the afferent channel with that in the efferent channel—in terms of metabolites removed from the afferent flow, and newly synthesized proteins in the efferent flow.

Devices of this kind can be used to detoxify a fluid such as blood, wherein the fluid comes into contact with the differentiated cells (e.g., hepatocytic cells) of the present invention under conditions that permit the cell to remove or modify a toxin in the fluid. Detoxification involves removing or altering at least one ligand, metabolite, or other compound (either natural and synthetic) that is usually processed by the liver. Such compounds include but are not limited to bilirubin, bile acids, urea, heme, lipoprotein, carbohydrates, transferrin, hemopexin, asialoglycoproteins, hormones such as insulin and glucagon, and a variety of small molecule drugs. The device can also be used to enrich the efferent fluid with synthesized proteins such as albumin, acute phase reactants, and unloaded carrier proteins. The device can be constructed so that a variety of these functions are performed, thereby restoring as many hepatic functions as are needed. In the context of therapeutic care, the device processes blood flowing from a patient in hepatocyte failure, and then the blood is returned to the patient.

Differentiated stem cells of the invention that demonstrate desirable functional characteristics in animal models (such as those described above) may also be suitable for direct administration to human subjects with impaired organ (e.g., liver) function. For purposes of hemostasis, the cells can be administered at any site that has adequate access to the circulation, typically within the abdominal cavity. For some metabolic and detoxification functions, it is advantageous for the cells (e.g., hepatocytes) to have access to the biliary tract. Accordingly, the cells are administered near the liver (e.g., in the treatment of chronic liver disease) or the spleen (e.g., in the treatment of fulminant hepatic failure). In one method, the cells administered into the hepatic circulation either through the hepatic artery, or through the portal vein, by infusion through an in-dwelling catheter. A catheter in the portal vein can be manipulated so that the cells flow principally into the spleen, or the liver, or a combination of both. In another method, the cells are administered by placing a bolus in a cavity near the target organ, typically in an excipient or matrix that will keep the bolus in place. In another method, the cells are injected directly into a lobe of the liver or the spleen.

The differentiated hepatic cells derived from liver stem cells of this invention can be used for therapy of any subject in need of having hepatic function restored or supplemented. Human conditions that may be appropriate for such therapy include fulminant hepatic failure due to any cause, viral hepatitis, drug-induced liver injury, cirrhosis, inherited hepatic insufficiency (such as Wilson's disease, Gilbert's syndrome, or α-antitrypsin deficiency), hepatobiliary carcinoma, autoimmune liver disease (such as autoimmune chronic hepatitis or primary biliary cirrhosis), and any other condition that results in impaired hepatic function.

Other differentiated cell types described herein can be used for therapy of a subject in need of treatment that would be ameliorated by the differentiated cell type. For example, chondrocytes can be used for cartilage repair (e.g., in osteoarthritis); osteocytes can be used for repairing bone loss such as occurs in osteoporosis; myoblasts can be used to treat degenerative muscle disorders such as a muscular dystrophy, muscle wasting associated with steroid therapy, muscle loss after tumor resection; cardiomyocytes can be used for therapy after myocardial infarction; adipocytes can be used for soft tissue repair such as in cosmetic or reconstructive surgery. For human therapy, the dose is generally between about 10⁹ and 10¹² cells, and typically between about 5×10⁹ and 5×10¹⁰ cells, making adjustments for the body weight of the subject, nature and severity of the affliction, and the replicative capacity of the administered cells. The ultimate responsibility for determining the mode of treatment and the appropriate dose may be provided by the skilled artisan, for example a physician or managing clinician.

5.8 Drug and Toxicological Screens

The undifferentiated adult liver stem cells of the invention, and the differentiated hepatocytic derivatives thereof, can be used to screen for factors (such as solvents, small molecule drugs, peptides, polynucleotides, peptoids, small non-nucleic acid organic molecules, small inorganic molecules, and the like) or environmental conditions (such as culture conditions or manipulation) that affect the characteristics of differentiated or undifferentiated cells of the hepatocyte lineage. For example, adult liver stem cells may represent the primary targets for oncogenic transformation of the liver (i.e. under the “stem cell theory of carcinogenesis”). Accordingly, the adult liver stem cells of the invention may be used to screen compounds and formulations for their cancer-causing potential.

Particular screening applications of this invention relate to the testing of pharmaceutical compounds in drug research (see, generally, In vitro Methods in Pharmaceutical Research, Academic Press, 1997; and U.S. Pat. No. 5,030,015). In this invention, adult stem cells (e.g., adult liver stem cells or adipocyte stem cells) that have differentiated to a differentiated cell lineage (e.g., a hepatocyte lineage, adipocyte lineage, osteocyte lineage, chondrocyte lineage, or myoblast lineage) play the role of test cells for standard drug screening and toxicity assays, as have been previously performed on cell lines (e.g., hepatocyte cell lines, adipocyte cell lines, osteocyte cell lines, chondrocyte cell lines, or myoblast cell lines) or primary cells in short-term culture (e.g., hepatocytes, adipocytes, osteocytes, chondrocytes, or myoblasts). Assessment of the activity of candidate pharmaceutical compounds generally involves combining the differentiated cells of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on liver cells, or because a compound designed to have effects elsewhere may have unintended hepatic side effects. Two or more drugs can be tested in combination (by combining with the cells either simultaneously or sequentially), to detect possible drug-drug interaction effects.

In certain useful applications, compounds are screened specifically for potential hepatotoxicity (see pp 375-410 of Castell et al. (1997) In Vitro Methods in Pharmaceutical Research, Academic Press). Cytotoxicity can be determined in the first instance by the effect on cell viability, survival, morphology, and leakage of enzymes into the culture medium. More detailed analysis is conducted to determine whether compounds affect cell function (such as gluconeogenesis, ureagenesis, and plasma protein synthesis) without causing toxicity. Lactate dehydrogenase (LDH) is a good marker because the hepatic isoenzyme (type V) is stable in culture conditions, allowing reproducible measurements in culture supernatants after 12-24 hour incubation. Leakage of enzymes such as mitochondrial glutamate oxaloacetate transaminase and glutamate pyruvate transaminase can also be used. Gomez-Lechon et al. ((1996) Anal. Biochem. 236: 296) describe a microassay for measuring glycogen, which can be applied to measure the effect of pharmaceutical compounds on hepatocyte gluconeogenesis.

Other methods to evaluate hepatotoxicity include determination of the synthesis and secretion of albumin, cholesterol, and lipoproteins; transport of conjugated bile acids and bilirubin; ureagenesis; cytochrome P450 levels and activities; glutathione levels; release of a-glutathione S-transferase; ATP, ADP, and AMP metabolism; intracellular K⁺ and Ca²⁺ concentrations; the release of nuclear matrix proteins or oligonucleosomes; and induction of apoptosis (indicated by cell rounding, condensation of chromatin, and nuclear fragmentation). DNA synthesis can be measured as [³H]-thymidine or BrdU incorporation. Effects of a drug on DNA synthesis or structure can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread (see pp 375-410 of Vickers (1997) In vitro Methods in Pharmaceutical Research Academic Press).

In certain other applications, stem cells (differentiated or undifferentiated) are used to screen factors that promote maturation of cells along the selected cell lineage (e.g., hepatocyte lineage, adipocyte lineage, osteocyte lineage, chondrocyte lineage, or myoblast lineage), or promote proliferation and maintenance of such cells in long-term culture. For example, a candidate maturation factor or growth factor is tested by adding the candidate factor to stem cells in different wells, and then determining any phenotypic change that results, according to desirable criteria for further culture and use of the cells.

### 6. EXAMPLES

6.1 General

Individual cell culture approaches have been developed that utilize specific calcium levels for the propagation of keratinocytes (Rheinwald and Green (1975) Cell 6: 331-343; Yuspa and Morgan (1981) Nature 293: 72-74), specific cell redox conditions affect the proliferation and lifespan of stem/precursor cells (Smith et al. (2000) Proc. Natl. Acad. Sci. USA 97:10032-10037; Studer et al. (2000) J. Neurosci. 20:7377-7383), or specific inactivation of poly (ADP-ribose) polymerase (PARP) to extend cellular lifespan (Vaziri et al. (1997) EMBO J. 16:6018-6033). To develop improved methods of culturing cells of clonal origin having stem cell phenotypes and that were derived from adult tissue (e.g., human liver stem cells, human adipocyte stem cells), multiple modifications were made to standard cell culture media and methods.

Previous reports about the development of fetal human or rodent liver hepatoblasts or precursor cells used the Dulbecco's modified MEM (DMEM) (Malhi et al. (2002) J. Cell Sci. 115:2679-2688) or a 1:1 mixture of DMEM and Ham's F12 (Kubota et al. (2000) Proc. Natl. Acad. Sci. USA 97:12132-12137). The calcium concentrations of these media (1.8 mM and 0.9 mM) are much higher than the K-NAC medium (0.09 mM) used in this study. The K-NAC medium also includes N-acetyl-L- cysteine and L-ascorbic acid-2-phosphate, which may maintain and promote the growth of stem/precursor and other somatic cells (Smith et al. (2000) Proc. Natl. Acad. Sci. USA 97:10032-10037; Hata et al. (1989) J. Cell Physiol. 138:8-16). Another difference is that our K-NAC medium supports the colony formation of adult human liver cells with stem cell phenotypes on plastics, whereas the other methods rely on irradiated autologous fetal liver cells (Malhi et al. (2002) J. Cell Sci. 115:2679-2688) or embryonic liver stroma feeders (Kubota et al. (2000) Proc. Natl. Acad. Sci. USA 97:12132-12137) for colony formation.

In the following examples, the isolation of human adult liver stem cells of clonal origin and capable of sustained growth is demonstrated. Adult human liver cell lines of clonal origin with stem cell phenotypes were isolated using a low calcium modified MCDB 153 medium supplemented with N-acetyl-L-cysteine, L-ascorbic acid-2-phosphate, and nicotinamide. These adult liver stem cells are characterized by high proliferation potential (at least 54 cumulative population doublings), anchorage- independent growth (5%), deficiency in gap junctional intercellular communication (GJIC), and the expression of hallmark liver stem (oval) cell markers. These adult liver stem cells expressed multiple markers including alpha-fetoprotein (AFP), vimentin and Thy-1. Furthermore, the transcription factor, Oct-4, previously reported to be exclusively expressed in pluripotent early embryo cells, embryonic stem cells and undifferentiated tumor cells, was also expressed in these human liver stem cell lines. These liver cells may divide symmetrically (1 serpiginous to 2 serpiginous shaped cells) or asymmetrically (1 serpiginous to 1 serpiginous and 1 cuboidal). Furthermore, after extensive growth in a modified Eagle's MEM, the liver stem cell phenotypes disappeared, i.e. became competent in GJIC, lost vimentin expression and the ability of cell migration to form cell “bridges” between micromass cell aggregates. Significantly, the human hepatoma cell line (Mahlava) was also found to express Oct-4, AFP and vimentin, and to be deficient in GJIC.

The primary human liver cell line from this study and its SV40 large T-antigen transformed clones did not show telomerase activity and eventually became senescent. An SV40 large T-antigen-expressing clone selected from soft agar growth, however, became immortal with activated telomerase activity. Thus, human liver cell lines with stem cell phenotypes can be derived from a small adult liver wedge biopsy.

6.2 Cell Culture Media

The medium used to develop the human liver stem/progenitor cell cultures is a modified MCDB 153 (Keratinocyte-SFM, GIBCO—Invitrogen Corporation) supplemented with 1 mM N-acetyl-L-cysteine (NAC) and 0.2 mM L-ascorbic acid 2-phosphate (Asc 2P) (referred to as K-NAC medium herein). The calcium concentration of this medium is 0.09 mM. The growth factors/hormones for this medium are rEGF (5 ng/ml), bovine pituitary extract (50 μg/ml) insulin (5 μg/ml), hydrocortisone (74 ng/ml) and 3,3′,5-triiodo-D.L.-thyronine (T₃) (6.7 ng/ml). NAC, a potent antioxidant found to promote the self-renewal of precursor cells (Smith et al. (2000) Proc. Natl. Acad. Sci. USA 97:10032-10037), is readily deacetylated in cells to yield L-cysteine, thereby enhancing the production of glutathione (De Flora et al (2001) Carcinogenesis 22:999-1013). Asc 2P is a stable precursor to provide ascorbic acid in cell culture (Hata et al. (1989) J. Cell Physiol. 138:8-16; Chepda et al. (2001) In Vitro Cell Dev. Biol. Anim. 37:26-30). Ascorbate can be antioxidative or pro-oxidative (i.e., reversible interconversion between ascorbate and dehydroascorbate) depending on its concentration and concentration of metal ions in a culture (Buettner (1988) J. Biochem. Biophvs. Meth. 16:2740). The medium is also supplemented with 5 mM of the poly ADP-ribose polymerase (PARP) inhibitor, nicotinamide. RPMI 1640 medium (GIBCO-Invitrogen) was also used in the initial tissue digestion and the growth of SV-40 large T-antigen immortalized human liver cells. The hepatoma cells were grown in Dulbecco's modified Eagle medium (GIBCO-Invitrogen Corporation) supplemented with 10% fetal bovine serum (FBS). Another modified Eagle's MEM (Chang et al. (2000) Somat. Cell Genet. 7: 235-53) was used in certain liver cell differentiation protocols.

6.3 Development of Clonal Liver Cell Culture

After obtaining formal consents from patients, normal parts of liver, from surgically resected specimens from two males with hemangioma (HL1, age 49; HL 2, age 32) and from one wedge biopsy of a male with abnormal liver function and hepatomelagy who underwent choledocholithotomy for common bile duct stones (HL3, age 53), were used for this study. Small pieces of tissue were first minced with scalpels and then digested in 12 ml of collagenase (275 u/ml) (Sigma Chemical Company C9891 Type 1A) in a 1:1 mixture of RPMI 1640 and the K-NAC medium supplemented with nicotinamide (5 mM). After a one-hour digestion at 37° C., the cells or cell aggregates were incubated in serum-free K-NAC medium with 5 mM nicotinamide for colony development. The epithelial colonies developed in 3 weeks and were isolated by the trypsin glass-ring method for further growth and characterization. The digestion solution and the initial growth medium contained antibiotics (100 units/ml; penicillin; 100 μg/ml streptomycin) and antimycotic (amphotericin B as fungizone, 0.25 μg/ml). All cell cultures were incubated at 37° C. in incubators supplied with humidified air and 5% CO₂.

6.4 Characterization of Proliferation Potential, Anchorage Dependence and Gap Junctional Intercellular Communication (GJIC)

The proliferation potential of cell clones was determined by cumulative population doubling level (cpdl) in continual subculture and growth from a known number of cells. The cpdl was calculated by using the formula [in (Nf/Ni)/In 2], where Ni and Nf are initial and final number respectively, and In is the natural log. The initial number of cells for each propagation determination was 1×10⁵ cells in a 75 cm flask. For anchorage-independent growth (AIG) assays, 5×10⁴ cells suspended in 3 ml soft agar (0.33%) were overlaid on a layer of hard agar (0.5%; 3 ml) in each of triplicate plates (6 cm). The liquid medium (K-NAC with 5 mM nicotinamide and 10% FBS; 3 ml) was added and renewed once every 3 days. After 3-4 weeks incubation, the number of colonies developed was scored under a microscope with the colony-containing dish on top of a plate with a grid.

The gap junctional intercellular communication was studied by the scrape loading/dye transfer technique described in Trosko et al. ((2000) Methods 20:245-264).

6.5 Immunohistochemical Staining and Western Blot Analysis

Both immunostaining and Western blotting were used to characterize gene expression for clonally derived liver cell culture. For immunostaining, the cells grown in 35 mm plates were washed with phosphate-buffered saline (PBS) and fixed with 4% paraformaldehyde in PBS. After rinsing with PBS, the cells were permeabilized (0.5% Triton® X-100, 2% BSA, and 0.05% NaN₃ in PBS) for 10 minutes. The cells were then incubated with the primary antibody diluted to the required concentration in PBS/Triton® X-100/BSA overnight at room temperature. After rinsing with PBS, the cells were incubated with a secondary antibody conjugated with FITC or Cy3 in PBS/Triton/BSA buffer for 1 hour at room temperature. For nuclear staining, the cells were washed with PBS before incubation with 4′6 diamidino-2-phenylindole (DAPI, Sigma Chemical Company, D 8417) in PBS (1 μg/ml) for 5 minutes. After thoroughly washing the cells with PBS, the phase image and fluorescence of cells was observed and recorded using a Nikon Elipse TE 300 microscope connected to a digital camera and computer. The procedure for Western blot analysis was similar to that described previously (Trosko et al. (2000) Methods 20:245-264). Monoclonal antibodies that recognize proteins reported to be expressed by “oval cells” were used in this study and were obtained from Sigma Chemical Company (monoclonal anti-vimentin clone V9 (Product No. V-9254); anti-α-fetoprotein (AFP) clone C3 (A-8452); anti-mouse thy 1.1 clone TN-26 (M-7898); anti-cytokeratin peptide 18 (C-1399); anti-cytokeratin peptide 19 clone A53-B/A2 (C-69301); anti-cytokeratin peptide 7 clone LDS-68 (C-6417); anti- cytokeratin peptide 8 clone M20 (C5301); and monoclonal anti-human serum albumin clone HAS-11 (A-6684)).

A monoclonal anti-SV40 large T-antigen (Ab-2, DPO2-200 uG; Calbiochem.) antibody was used to detect T-antigen expression in transformed clones. The secondary antibody was an anti-mouse IgG (Fc-specific) developed in goat and conjugated with FITC.

The procedure for Western blot analysis has been described previously (Trosko et al. (2000) Methods 20:245-264). Briefly, the proteins were extracted from cells with 20% SDS and 1 mM phenylmethylsulfonyl fluoride (PMSF). Equal amounts of proteins (20 μg/lane) were separated by 12.5% SDS-PAGE and transferred from the gel to PVDF membranes. Immunoblotting was carried out with appropriate antibodies and immunoreactive protein complexes were detected using an ECL™ detection reagent.

6.6 Immortalization of Liver Cells by Transfection with SV40 Large T-Antigen

Early passage cells of a cell line obtained from this study, HL1-1, were plated in three 60 mm plates (1×10⁶ per plate). After overnight incubation, the cells were transfected with a plasmid carrying an origin-defective SV40 genome expressing the wild-type large T-antigen (PRNS-1, a gift from Johng S. Rhim, Uniformed Services University of the Health Sciences, Bethesda, Md.) (Sun et al. (1999) Cancer Res. 59: 6118) by Lipofectamine™ (Life Technologies, Inc., Gaithersburg, Md.) After 4 days incubation in the K-NAC medium containing 5 mM nicotinamide and 10% FBS, the cells were selected by incubation in 0.4 mg/ml G418 for 10 days. Surviving actively proliferating colonies were isolated by the trypsin glass ring method and propagated to accumulate a sufficient number of cells for storage in liquid nitrogen. These cells were then used for determination of proliferation potential and other characterizations. Large colonies of these cells developed in soft agar were also isolated to determine if their proliferation potential was different from the parental cells.

6.7 Telomerase Assay

The LightCycler TeloTAGGGhTERT Quantification kit (Roche Molecular Biochemicals, Sandhofer Strasse, Germany) was used to detect mRNA, encoding for human telomerase catalytic subunit hTERT, using the LightCycler instrument. A human gastric epithelial cell line, AGS, was used as a positive control.

6.8 Isolation of Human Liver Cell Colonies with Sustained Growth

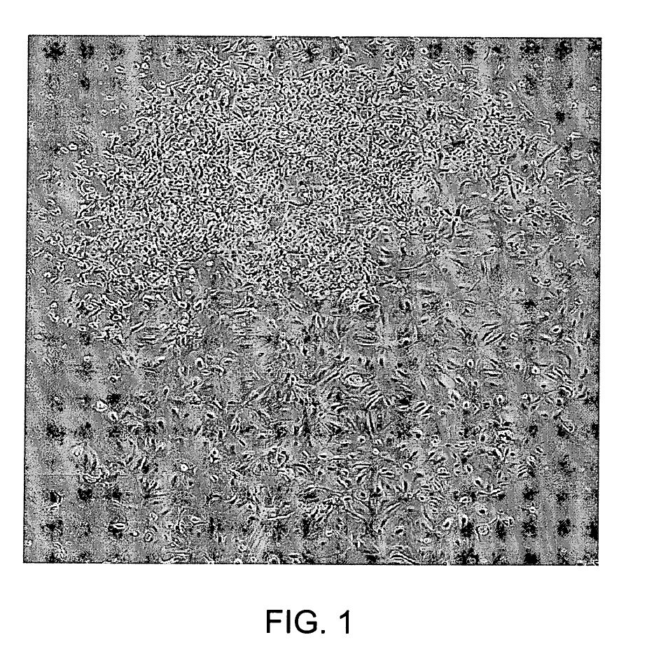

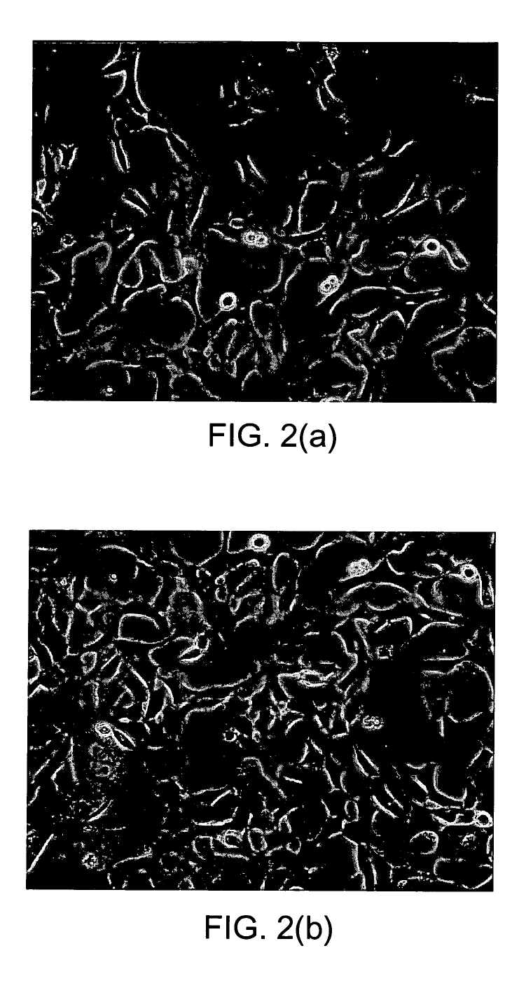

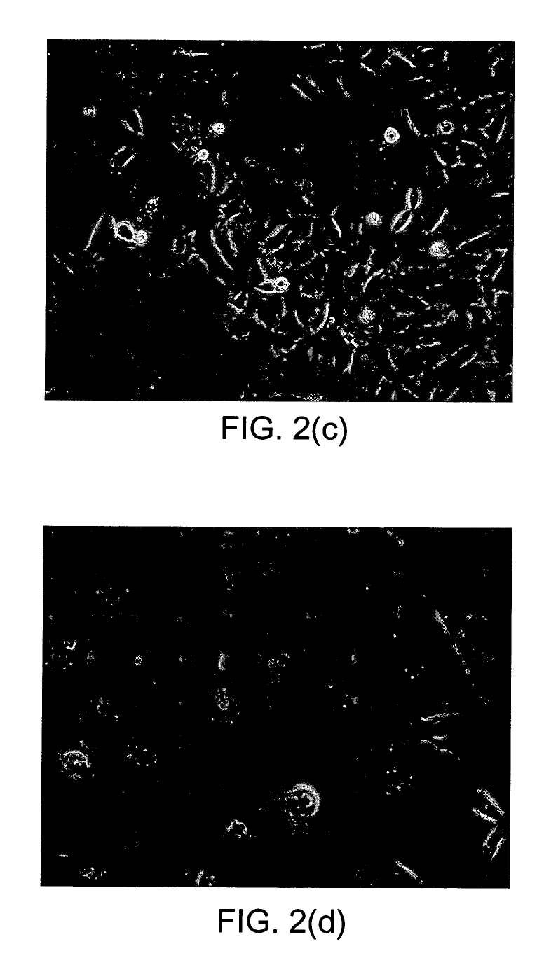

Three liver biopsies from 3 different patients were used in this study. These tissue samples were only about 500 mg in quantity. By using the serum-free and low calcium K-NAC medium, the growth of fibroblasts was inhibited. For the first experiment using a small piece of liver tissue, only one epithelial colony with sustained growth was isolated. This colony (HL1-1) when first observed in early stage contained only about 30 cells. After further growth, this colony actually gave rise to half a colony of actively proliferating cells of small size and half a colony of large-sized cells (FIG. 1). The small sized cells were epithelial-like whereas the large cells contained multiple nuclei similar to hepatocytes (FIGS. 2(a)-2(d)). The cell line from this colony was further characterized as described below.

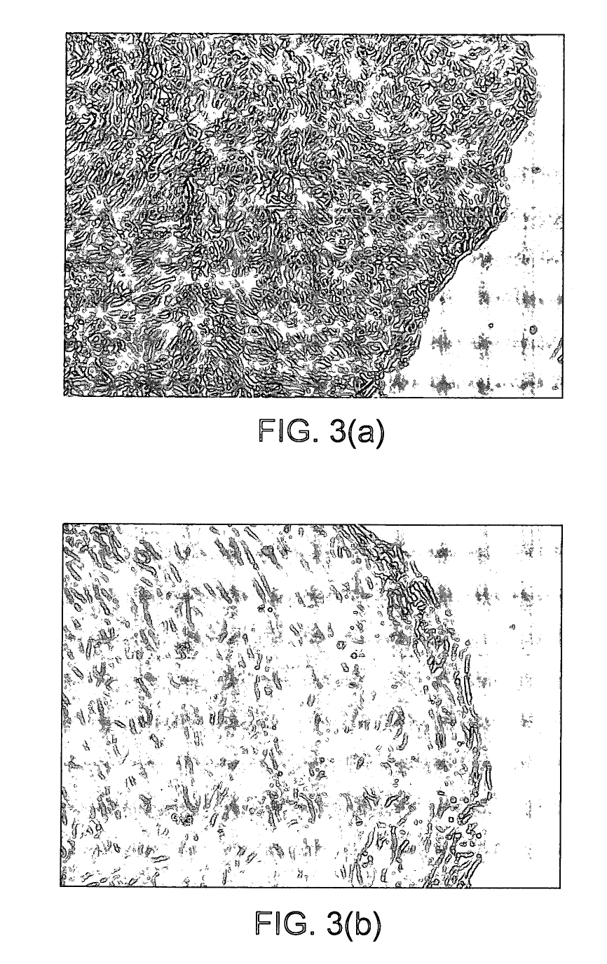

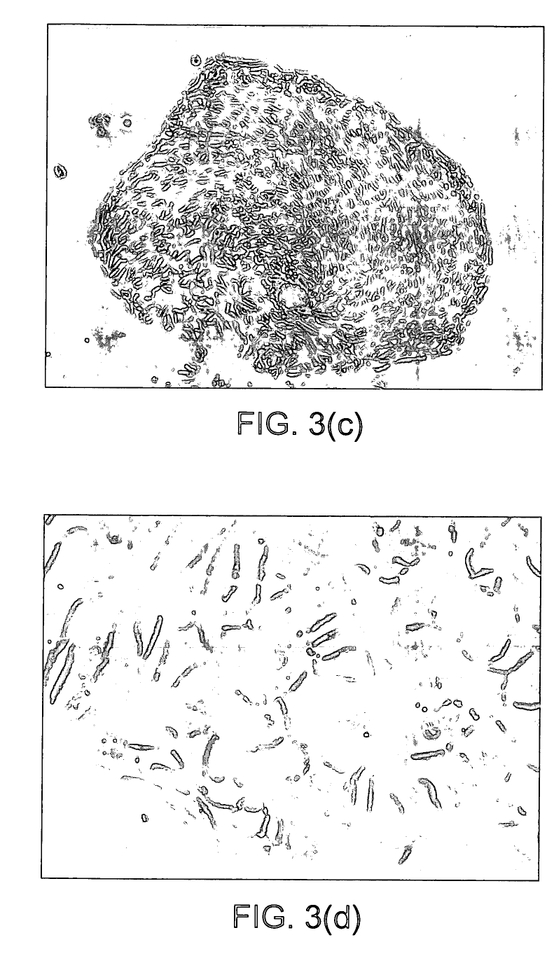

In the second and third experiments, multiple colonies were isolated (6 and 3 respectively for HL2 and HL3). These epithelial colonies had restricted smooth outside boundaries (FIGS. 3(a)-3(d)) that were similar to Type 1 human breast epithelial cells with stem cell characteristics (Kao et al. (1995) Carcinogenesis 16:531-538), for example, in colony morphology. After isolation and propagation in serum-containing medium, HL2 and HL3 cell grew like HL1-1 cells and did not form colonies with restricted outside boundaries.

6.9 Symmetric and Asymmetric Cell Divisions of Serpiginous Cells

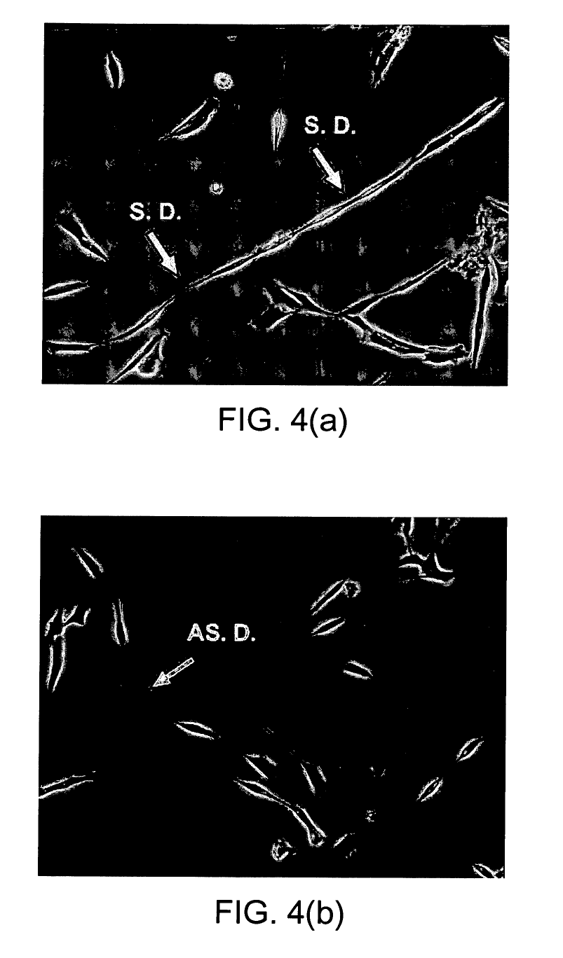

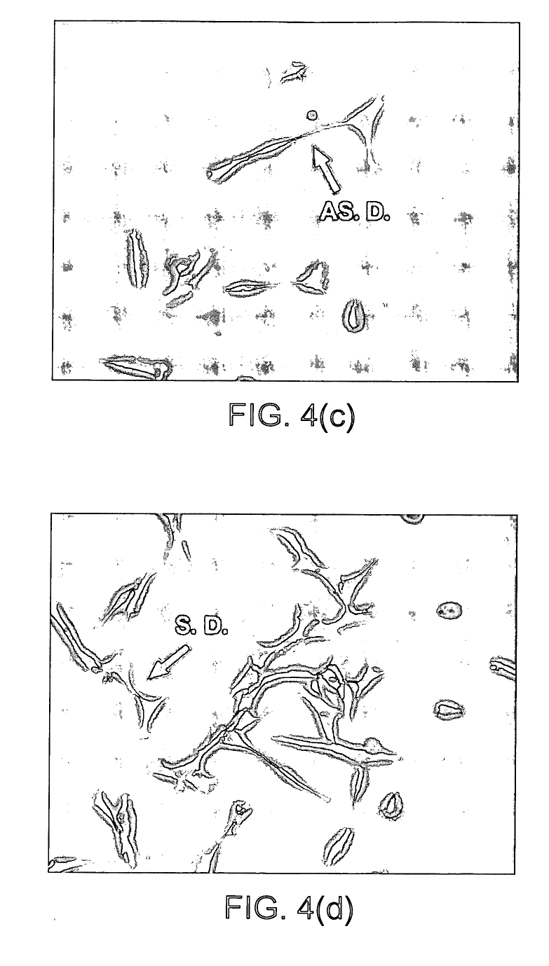

As described above, some stem cells or precursor cells may appear as serpiginous-shaped cells. Although the cells in the initial colony of HL1-1 were typically epithelial in morphology, e.g., cuboidal, the majority of the cells may appear as serpiginous cells especially when growing at low cell density or in growth factor- deprived medium. These serpiginous cells can divide symmetrically to give rise to two serpiginous cells or divide asymmetrically to give rise to one serpiginous cell and one cuboidal cell (FIGS. 4(a)-4(d)).

6.10 Proliferation Potential and Colony-Forming Ability

Although the liver cell colonies were initially developed in serum-free medium, these cells were found to grow better in the K-NAC medium supplemented with 5 mM nicotinamide and 10% FBS. This serum-containing medium was used in experiments to determine the proliferation potential and colony-forming ability.

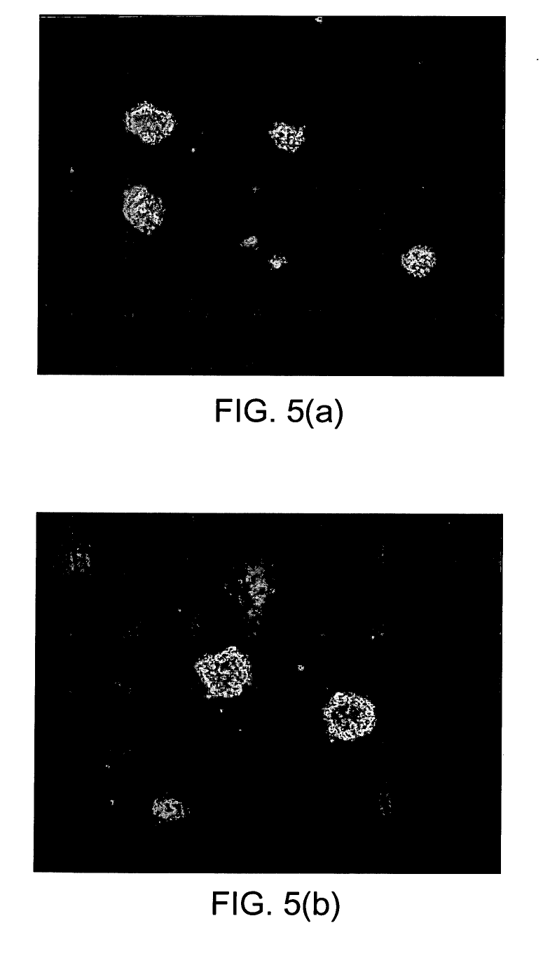

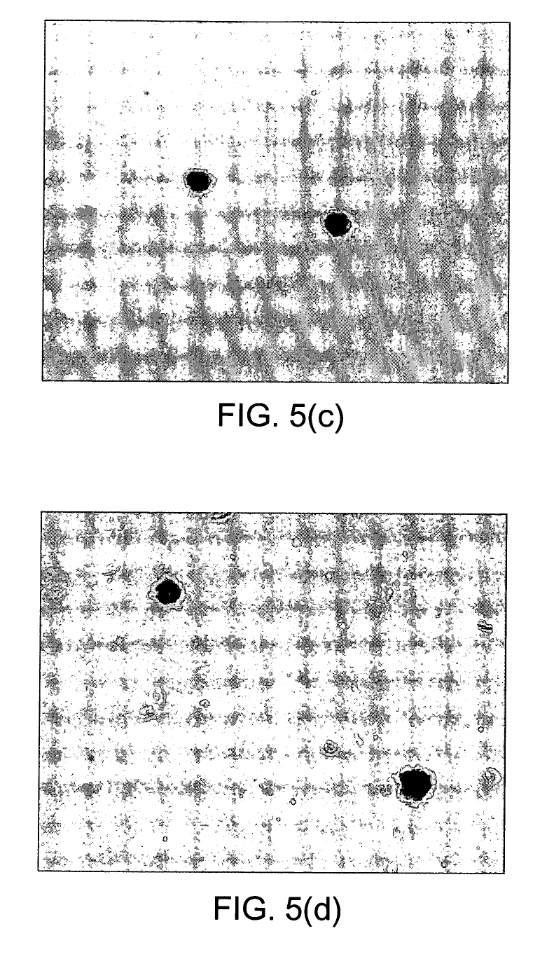

The cumulative population doubling levels (cpdl) for 3 clones derived from 3 different patients were determined. The clones were 49, 32, and 54 for HL1-1, HL2-3 and HL3-1 respectively. For HL1-1, the ability for anchorage-independent growth (FIGS. 5(a)-5(d)) was 5.5% and 4.6% in two separate experiments, in contrast to 0.24% for the normal human skin fibroblasts (MSU-2) growing in a modified Eagles MEM (Chang et al. (1981) Somat. Cell Genet. 7: 235-53) (1.8 mM calcium) with 10% FBS. On plastic surface, the colony-forming efficiency of HL1-1 is 10.7%.

6.11 Gap Junctional Intercellular Communication (GJIC)

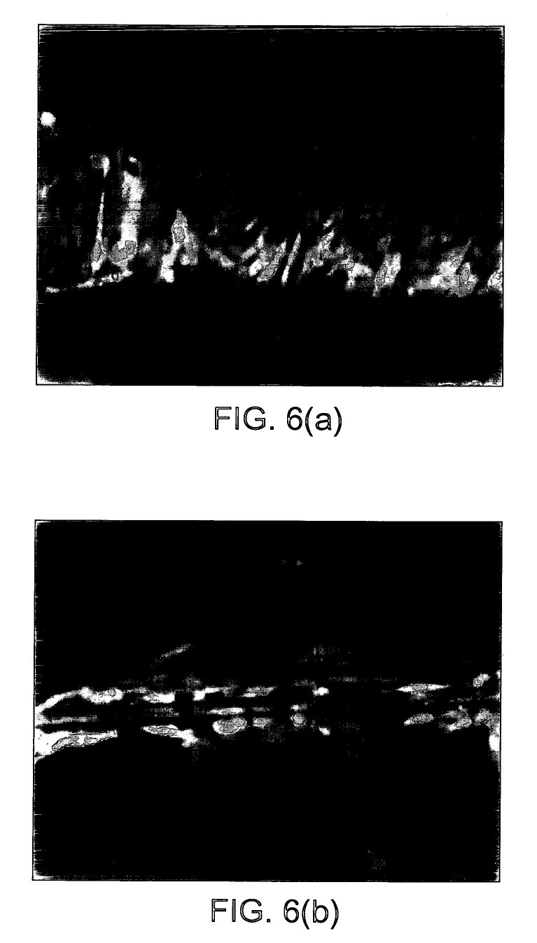

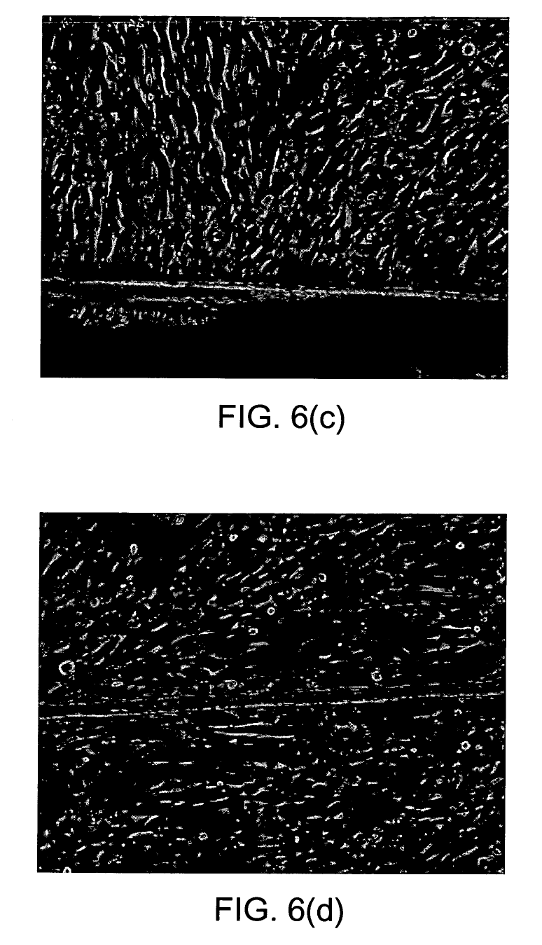

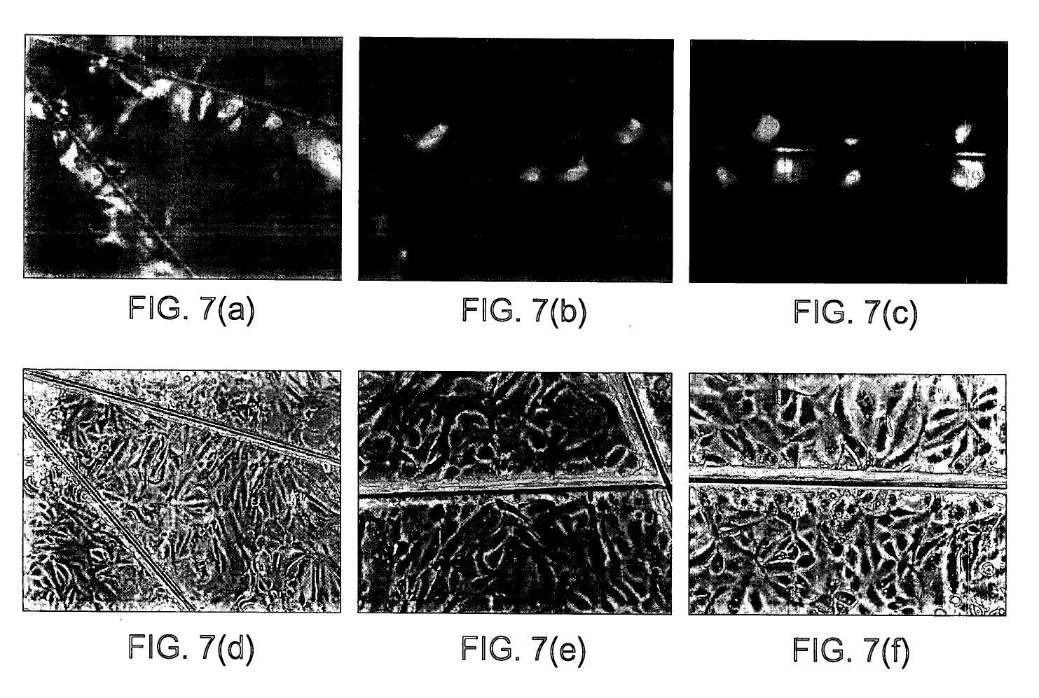

The ability of cells for GJIC in confluent culture or cell colonies was studied by the scrape-loading dye transfer technique. The results indicated that HL1-1 cells were deficient in GJIC (FIGS. 6(a)-6(d)) compared to the normal cells that are competent in GJIC. The epithelial clones developed in experiments 2 and 3 (HL8 and HL12) were also found to be deficient in GJIC (FIGS. 7(a)-7(f)).

6.12 Expression of Oval Cells Markers Studied by Immunostaining and Western Blotting

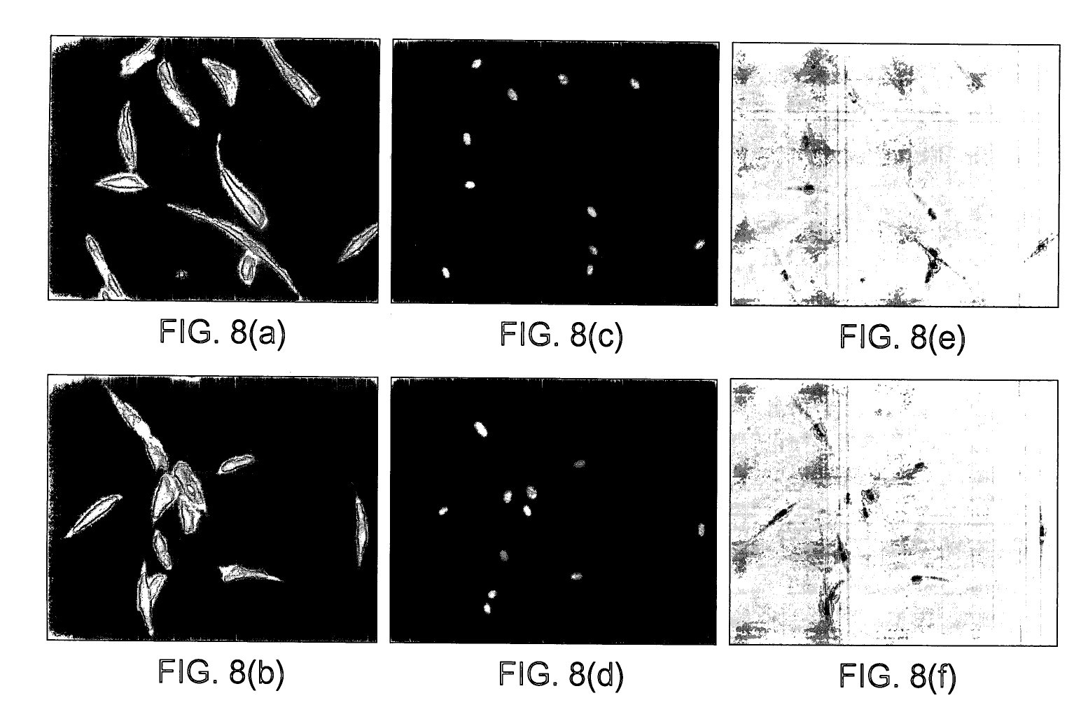

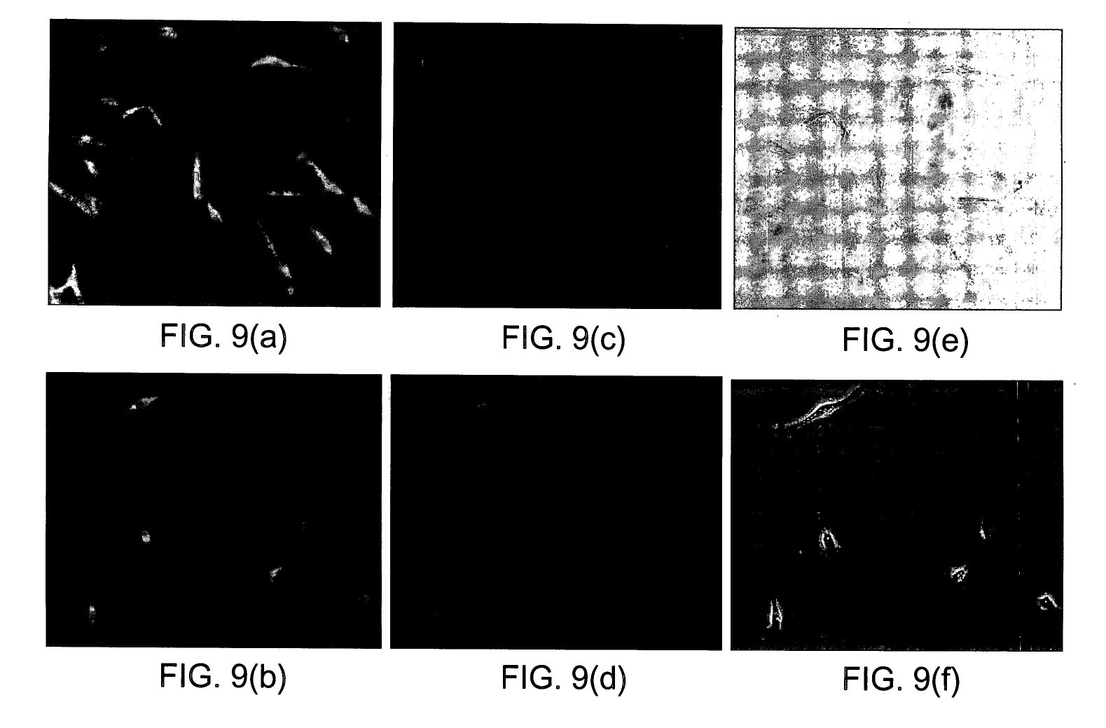

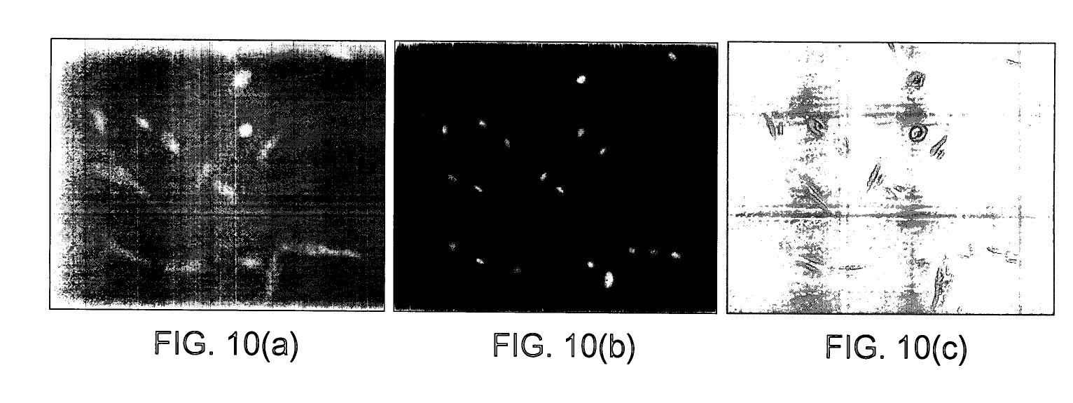

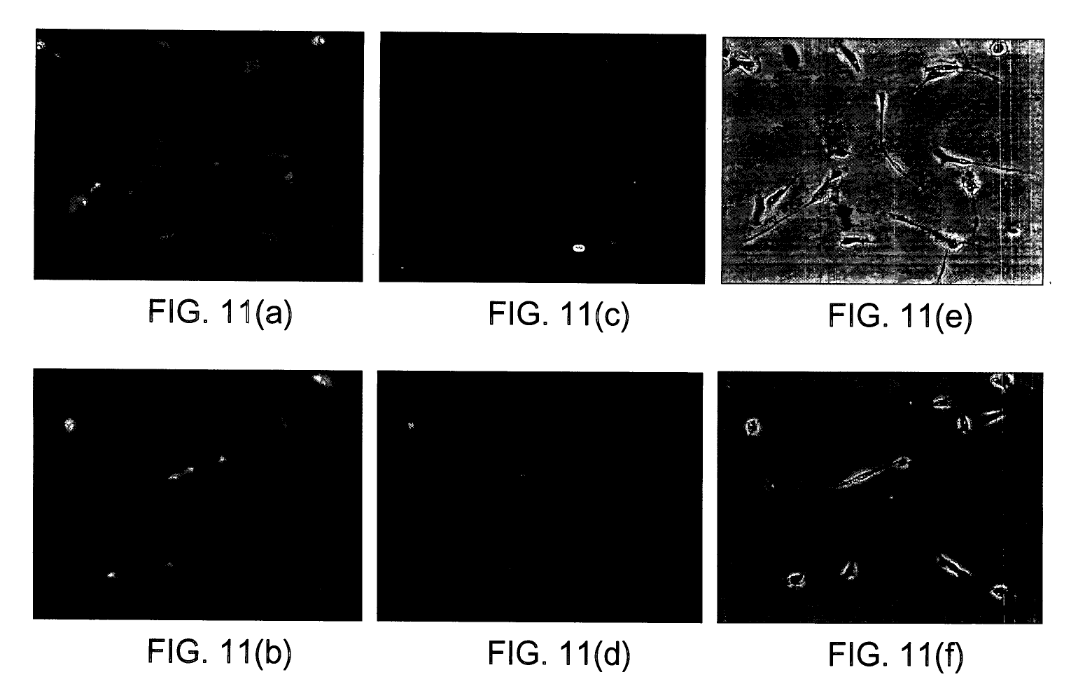

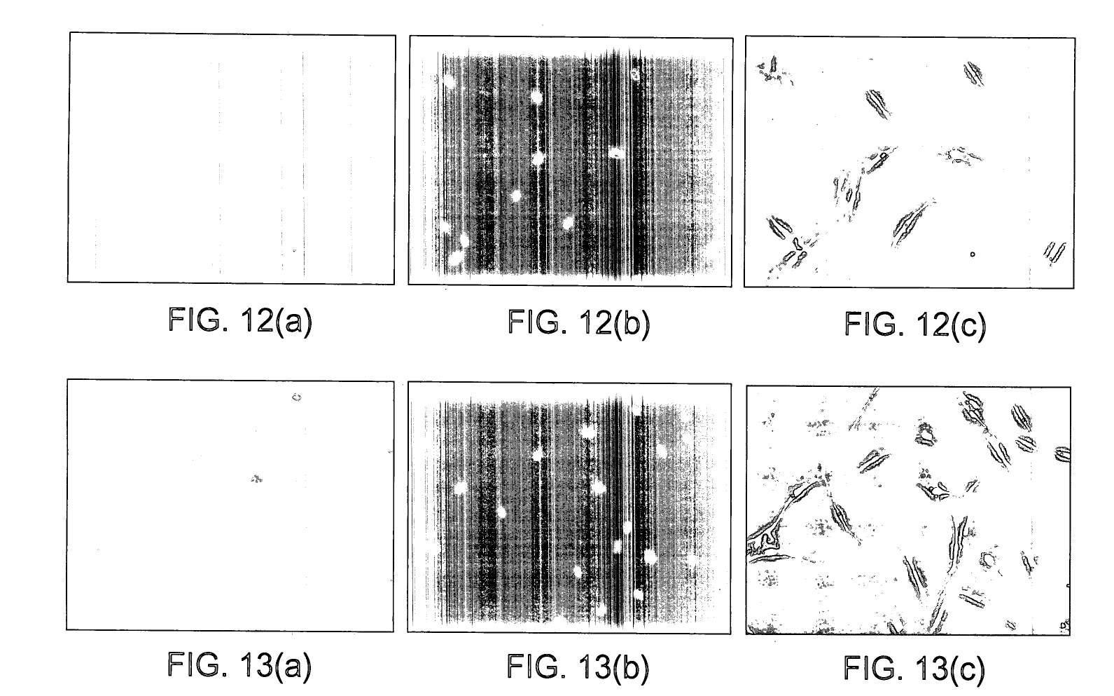

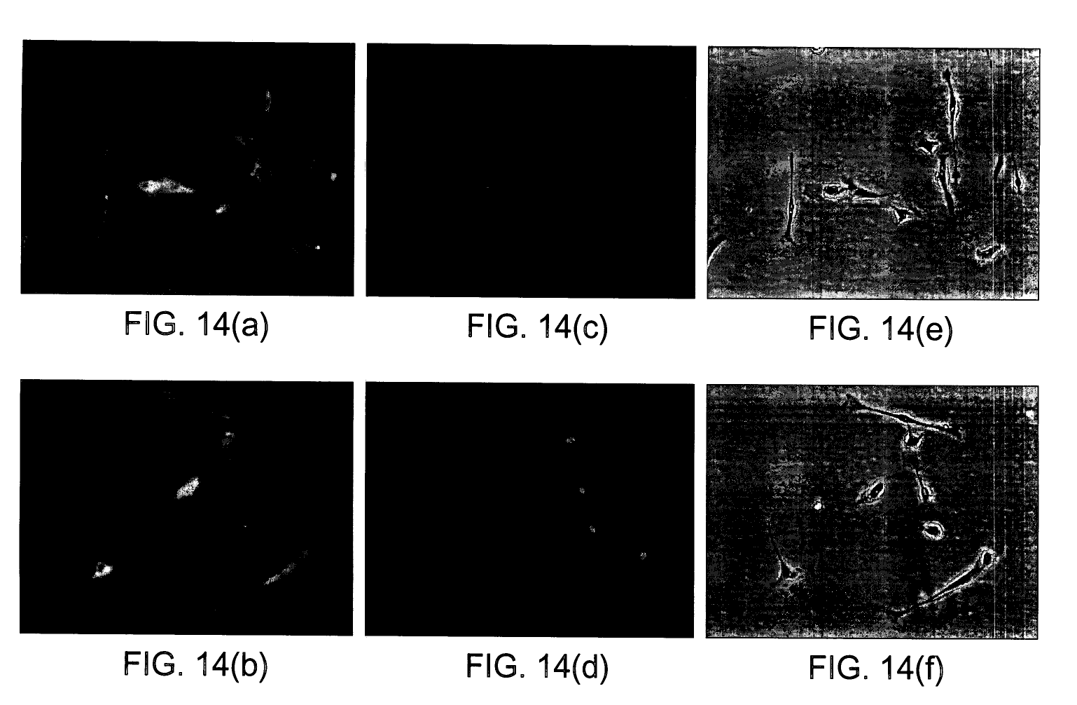

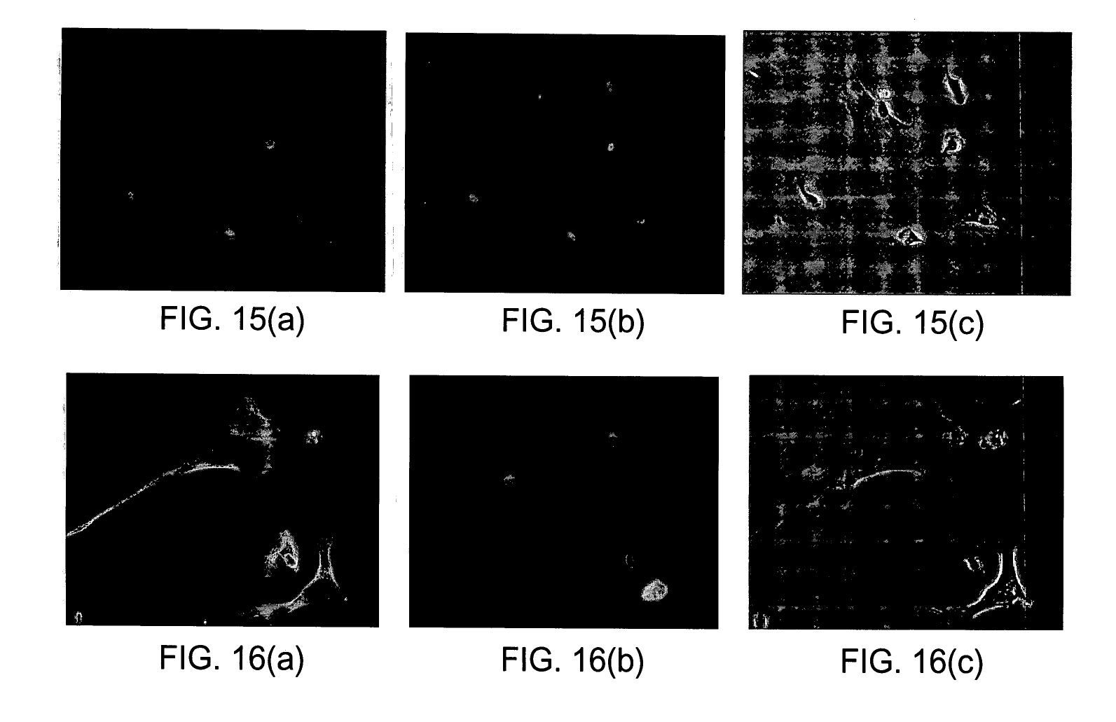

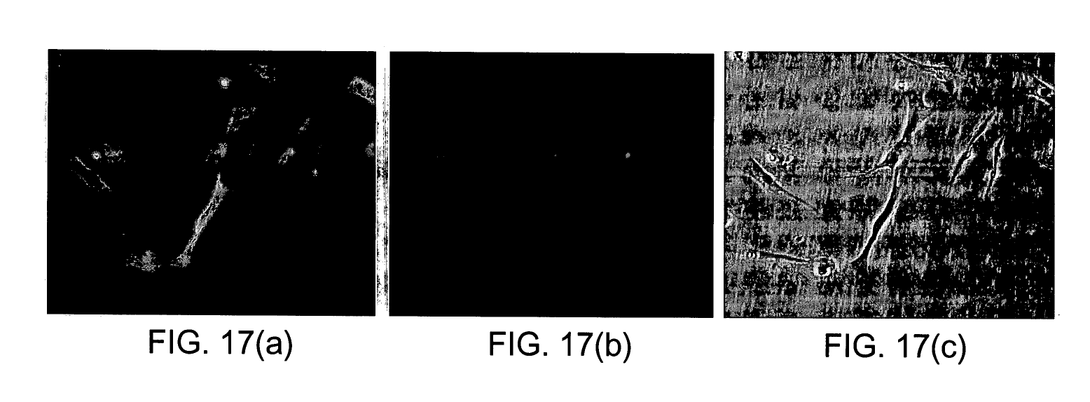

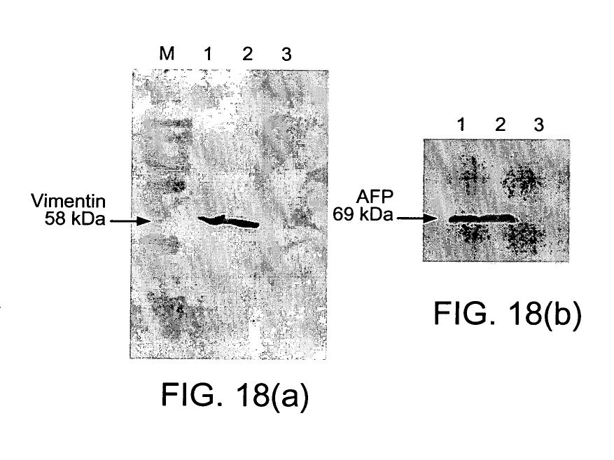

Vimentin, α-fetoprotein and thy-i have been shown to be specifically expressed in liver oval cells (Alison (1998) Curr. Opin. Cell Biol. 10:710-715). Experiments were performed to examine the similarities of cells cultured as described herein to authentic liver oval cells. By immunohistochemical staining, the vimentin, α-fetoprotein and thy-1 were found to be expressed in HL1-1 cells (FIGS. 8(a)-8(f), 9(a)-9(f), and 10(a)-10(c)), with strongest expression for vimentin and weaker expression for Thy-1. Cytokeratin, 7, 8, 18, and 19 were also weakly expressed in HL1-1 (FIGS. 11(a)-11(f), 12(a)-12(c), 13(a)-13(c), and 14(a)-14(f)). The major hepatocyte protein, albumin, was not detectible in these cells. Expression of vimentin, α-fetoprotein, and thy-1 was also found in HL2-3 (FIGS. 15(a)-15(c), 16(a)-16(c), 17(a)-17(c)), HL3-1, and HL3-2 clones. For HL2-3, the Thy-I expression is as strong as vimentin. The expression of vimentin and α-fetoprotein for HL1-1 was also confirmed by Western blot analysis (FIGS. 18(a) and 18(b)). The transcription factor Oct-4 is also expressed in the HL1-1 cells (Trosko et al. (2004) Proc. Am. Assoc. Cancer Res. (In press), as well as many other human adult stem cells.

6.13 Phenotypic Changes of Cells in a High Calcium Medium

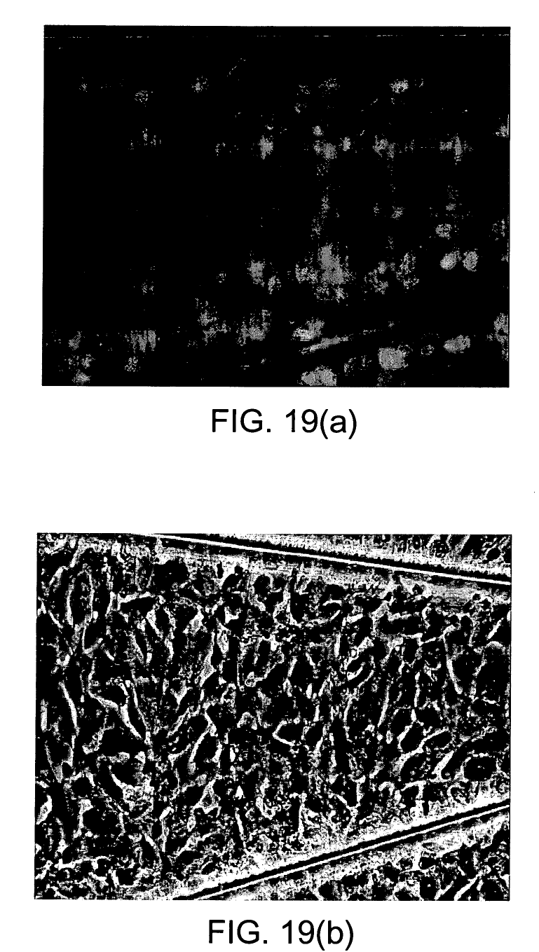

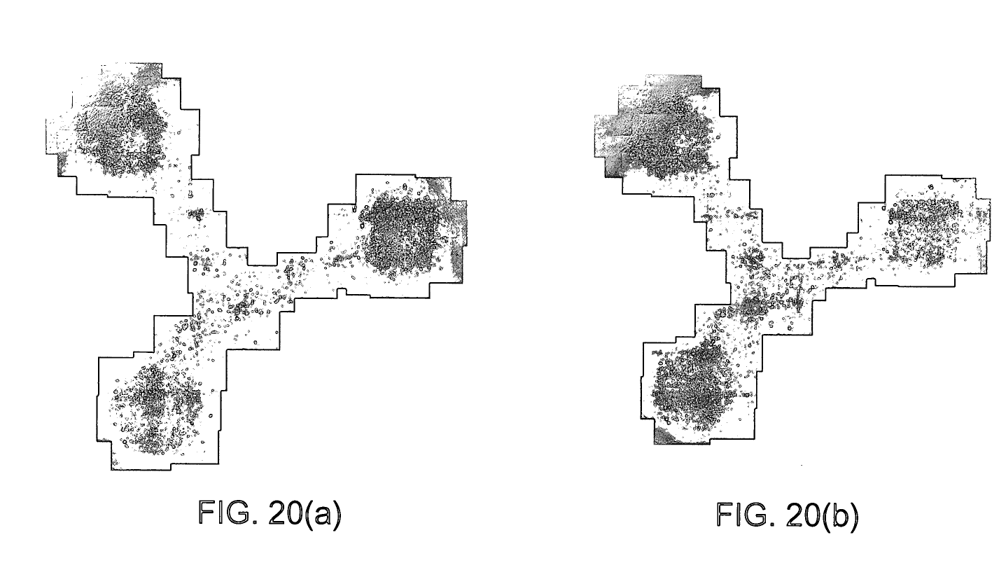

In order to achieve controlled differentiation of the isolated clonal adult human liver cell line HL1-1, cells were grown in a modified Eagle's MEM with high calcium (1.8 mM), (Chang et al. (1981) Somat. Cell Genet. 7: 235-53). Growth in this medium resulted in differentiation of the HL1-1 line, such that most cells changed cell morphology (larger, some with multiple nuclei) and other phenotypes. These altered mature hepatic cell phenotypes include the competence in GJIC (FIGS. 19(a) and 19(b)), and the loss of vimentin expression (data not shown). Without committing to any theory, the latter may be related to the loss of cell mobility to form cell “bridges” between micromass cell aggregates shown by the same population of cells grown in the K-NAC medium (FIGS. 20(a) and 20(b)).

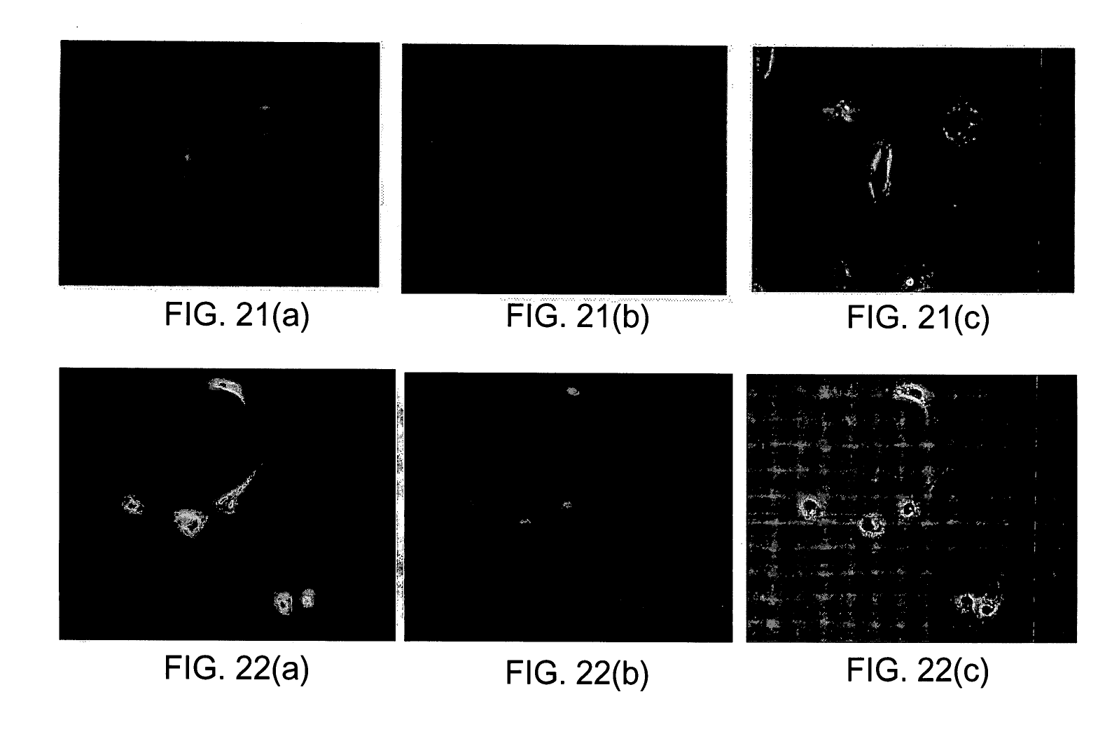

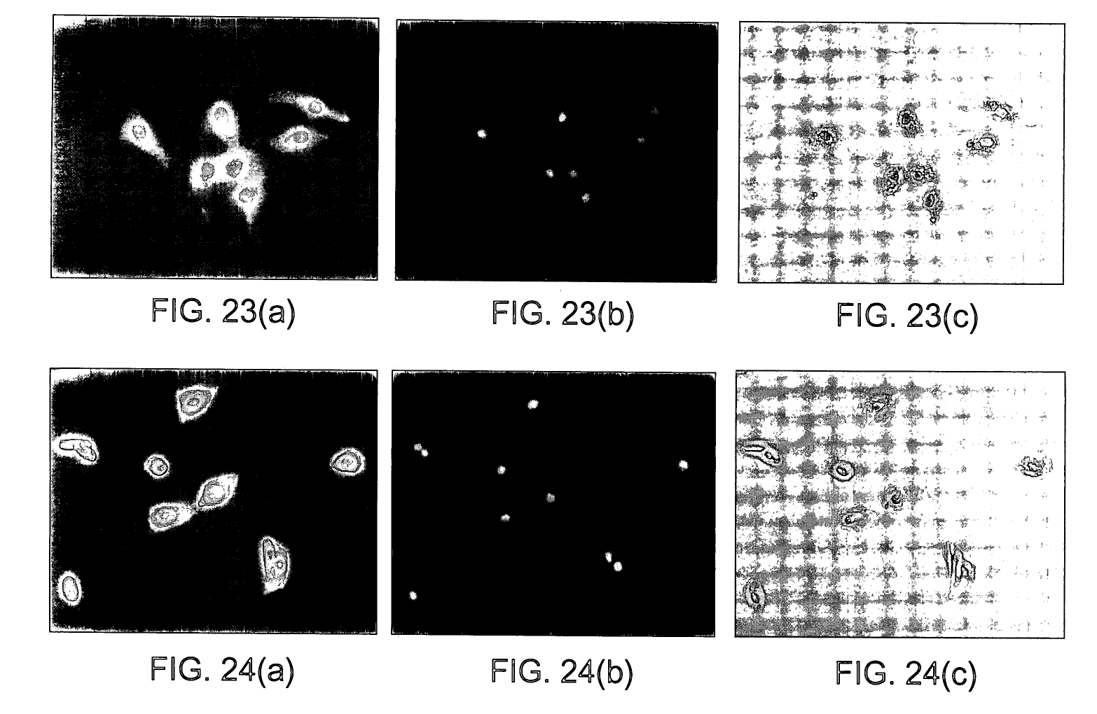

Furthermore, cells of the immortal liver cell line L1SV1A1, derived from HL1-1 (described infra), that were cultured in the modified Eagle's MEM (Chang et al. (1981) Somat. Cell Genet. 7: 235-53) containing hepatocyte growth factor (HGF) (He et al. (2003) Differentiation 71: 281-90) (20 ng/ml) expressed the hepatocyte protein, albumin, as detected by immunostaining (FIGS. 21(a)-21(c)). Albumin was detectable at 33 days but not at 22 days after the initiation of induction. Accordingly, both the adult human stem cell HL1-1 and its immortalized derivative, H1SV1A1 could be induced to undergo liver-specific differentiation in the modified Eagle's MEM with high calcium.

These data demonstrate that an adult stem cell line can be cultured and differentiated into a specific cell type. In particular, these data demonstrate that an adult stem cell line cultured using the new medium described herein (e.g., using culture conditions that include low calcium) retain their ability to differentiate into a specific cell type. These data also demonstrate that an adult stem cell line can be immortalized and retain the ability to differentiate in response to the appropriate conditions.

6.14 Phenotype Similarities of Human Adult Liver Stem/Precursor Cells and Hepatoma Cell Line

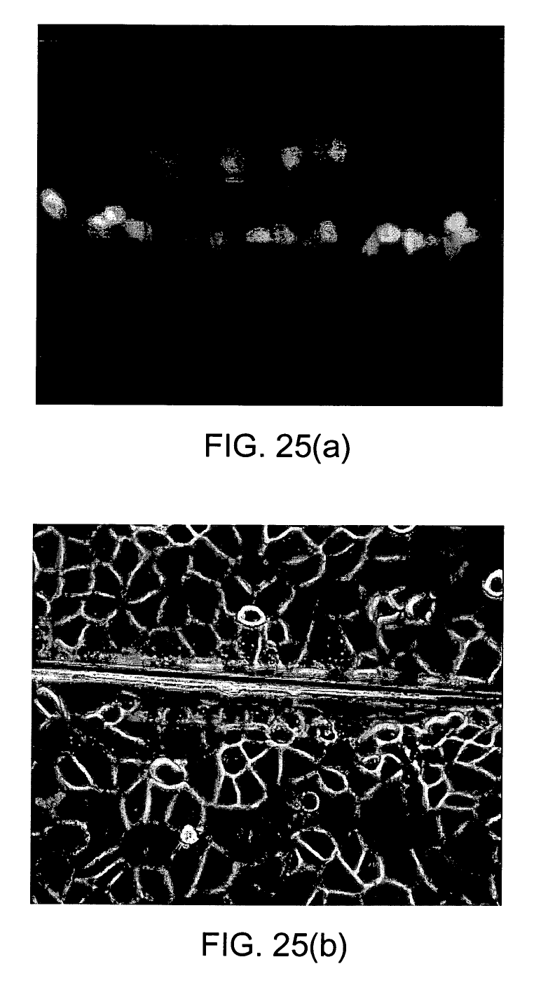

The specific liver oval cell markers vimentin, α-fetoprotein, and Thy-1, were found to be strongly expressed in a hepatoma cell line, Mahlava, by immunostaining study (FIGS. 22(a) 2 22(c), 23(a)-23(c), and 24(a)-24(c)). Furthermore, this hepatoma cell line also expressed Oct-4 (Trosko et al. (2004) Proc. Am. Assoc. Cancer Res. (In press), and was deficient in GJIC (FIGS. 25(a) and 25(b)). Thus, there is a similarity in phenotypes between this hepatoma cell line and the cell lines isolated from this study.

This further demonstrates that the methods described herein can be used to isolate and propagate stem cells derived from adult tissue that share properties with cancer cells or cancer stem cells (Dick (2002) Proc. Natl. Acad. Sci. US 100: 3547; Al-Hajj et al. (2003) Proc. Natl. Acad. Sci. US 100: 3983).

6.15 Immortalization of Human Liver Stem/Precursor Cells by SV40 Large T-Antigen

The HL1-1 cells were transfected with a plasmid carrying an origin-defective SV40 genome expressing the wild type large T-antigen (pRNS-1). Four days after transfection, the cells were selected with 0.4 mg/ml G418. Ten days after the selection, 3 surviving colonies were isolated. Two of the colonies (L1SV1 and L1SV2) with SV40 large T-antigen expression were propagated to determine their proliferation potential. Similar to the parental cells, L1SV1 cells were deficient in GJIC and expressed the oval cell markers, vimentin and α-fetoprotein (data not shown). Both clones eventually became senescent (L1SV1 at 54 cpdl; LiSV2 at 40 cpdl).

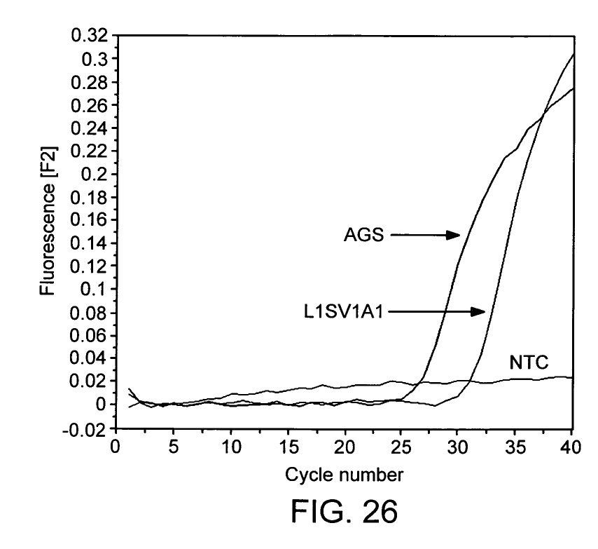

Anchorage-independent growth (AIG) was also examined. In these experiments LISVI cells were plated in soft agar. One large AIG colony was isolated for continuous growth. Different from the parental L1SV1 or HL1-1 cells, this clone (L1SV1A1) eventually became immortal (more than 100 cpdl) with activated telomerase activity (FIG. 26). These data demonstrate that adult stem cells can be immortalized using large T-cell antigen.

6.16 Human Clonal Adult Liver Stem Cells and Immortalized Derivatives

The HL1-1 colony isolated from the first experiment (section 5.7, supra) contained both actively proliferating epithelial cells and larger multinucleated cells (a phenotype of mature hepatocytes). This pattern indicates that undifferentiated actively proliferating cells could give rise to those large more differentiated cells and that this clone might be a good candidate for adult human stem/precursor cells. Upon further characterization, this clone and other clones were, indeed, found to possess many stem cell phenotypes. First, these cells showed high proliferation potential (HL1-1, cpdl=9; HL2-3, cpdl=32; HL3-1, cpdl=54). Considering that the whole human body contains about 100 trillion cells (less than 47 cpdl from one cell), the 49 and 54 cpdl represent a tremendously large number of cells and great proliferative capacity. Second, these cell clones are deficient in gap junctional intercellular communication similar to other adult human stem cells (Chang et al. (1987) Cancer Res. 47:1634-1645); Kao et al. (1995) Carcinogenesis 16:531-538; Matic et al. (2002) J. Invest. Dermatol. 118:110-116; Grueterich et al. (2002) Arch. Opthalmol. 120:783-790). Third, similar to human breast epithelial cell type with stem cell characteristics (Chang et al. (2001) Radiation Res. 155:201-207) and human mesenchymal stem cells derived from adipose tissues (Lin et al. (2004) Int. Soc. Stem Cell Res. Meeting) the HL1-1 cells were capable of anchorage- independent growth. Fourth, similar to some stem/precursor cells (Zulewski et al. (2001) Diabetes 50:521-533; Tang et al. (2001) Science 291:872-875), the liver cell lines isolated from this study are serpiginous in morphology especially when they are growing at low cell density or in growth factor-derived medium. Fifth, these liver cell lines express oval cell markers, i.e., vimentin, α-fetoprotein, thy-1, and the embryonic stem cell marker, Oct-4 that are also expressed in other adult human stem cells. In this study, the in vitro immortalized L1SV1A1 cell line was found to express the hepatocyte protein, albumin after treatment with HGF. The parent HL1-1 cells could be similarly induced to become hepatocytes.

Immortalization of human adult or fetal hepatocytes, but not stem/precursor cells, has been reported (Pfeifer et al. (1993) Proc. Natl. Acad. Sci. USA 90:5123-5127; Wege et al. (2003) Gastroenterol.124:432-444). In this study, the liver cell line having liver stem/precursor cell phenotypes was transfected with SV40 large T-antigen and succeeded in obtaining an immortal cell line with activated telomerase activity.

Stem cells are believed to be targets for oncogenic transformation (Chang et al. (2001) Radiation Res. 155:201-207). Indeed, there is evidence that mouse oval cells give rise to hepatocellular carcinoma (Dumble et al. (2002) Carcinogenesis 23:435-445). Oval cells are also found in human liver disease conditions caused by alcohol, hepatitis C virus, and in hemochromatosis (Lowes et al. (1999) Am. J. Pathol. 154:537-541), which are associated with increased incidence of hepatocellular carcinoma or cholangiocarcinoma (Tsukuma et al. (1993) N. Engl. J. Med. 328:1797-1801; Deugnier et al. (1993) Gastroenterol. 104:228-234; Prior (1988) Alcohol Alcohol 23:163-171). In the present study, the human hepatoma cell line, Mahlava, and the clonal adult liver cell lines with stem cell phenotypes share certain phenotypes, i.e., the deficiency in gap junctional intercellular communication, the expression of vimentin, α-fetoprotein, thy-1, and Oct-4. These findings strongly support the stem cell theory of carcinogenesis.

The information provided herein study shows that human cell lines with stem cell characteristics can be developed from a small biopsy of adult human tissue (e.g., liver). Such cell lines are potentially useful for cell transplantation, tissue engineering of bioartificial organs, and gene therapy in therapeutic treatment of patients suffering liver failure. Cells of these lines can be trans-differentiated to become other cell types and used for treatment of other non-liver diseases. For example, such cells can be trans- differentiated to pancreatic endocrine cells for treatment of diabetics (Yang et al. (2002) Proc. Natl. Acad. Sci. USA 99:8078-8083; Ber et al. (2003) J. Biol. Chem. 278:31950-31957). Since liver stem cells can be target cells for carcinogenesis, the cells described herein can be used to develop an in vitro model to study the mechanism of human liver carcinogenesis.

6.17 Mesenchymal Stem Cells Derived from Adipose Tissue

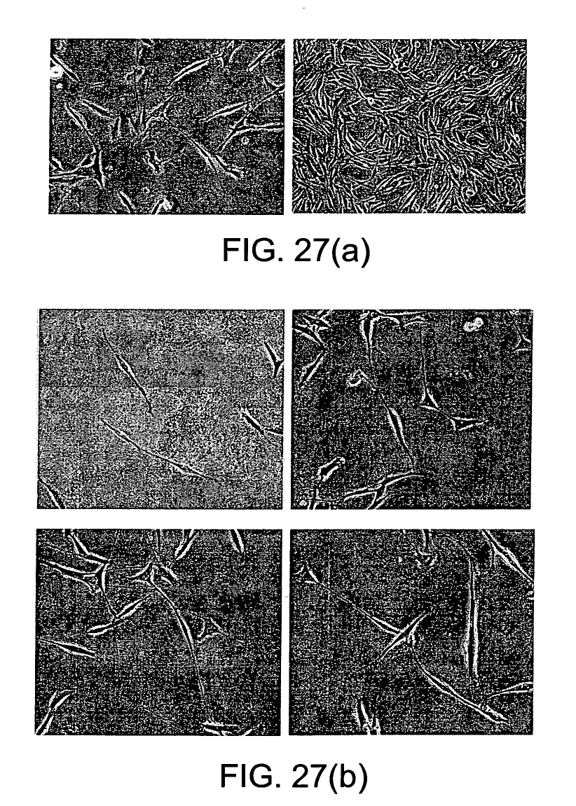

A new method of culturing stem cells derived from liver (liver stem cells) is described supra. The method can be adapted to isolate additional types of stem cells from adipose tissues, i.e., mesenchymal stem cells. In these experiments, fat tissue obtained from liposuction procedures or minced fat tissues obtained by surgery were washed with phosphate buffered saline (PBS) 4-5 times on top of a sterile gauze placed on a beaker to remove most red blood cells. The processed lipoaspirates (PLA) or adipose tissues suspended in PBS of equal volume were then centrifuged at 200×g for 10 minutes. The washing was repeated once. The upper fraction of adipose tissue was then digested using collagenase and dispase in Dulbecco's Modified Eagle Medium (DMEM) supplemented with N-acetyl-L-cysteine (NAC, 2 mM), L-ascorbic acid 2-phosphate (Asc-2P, 0.2 mM), and antibiotics/antimycotic (penicillin, streptomycin, and amphotericin) overnight at 37° C. with rocking or rotation. As an example, 8 g of tissue was digested in a 50 ml tube containing 40 ml DMEM with 40 mg collagenase and 32 units dispase. The digested PLA or tissue was then centrifuged to collect the cells. The cell pellet was then washed in DMEM, centrifuged, and the resulting cell pellet was dispersed and incubated in DMEM with 10% FBS, NAC, Asc-2-P, antibiotics, and antimycotics on plastic culture flasks. After culturing the cells overnight, the unattached cells were removed by washing three times with PBS and then incubated in a modified MCDB 153 medium containing NAC (2 mM), Asc-2 P (0.2 mM) (this medium is referred to as “K-NAC” medium) and 5% FBS. Adipose-derived cells developed under these conditions possess mesenchymal stem cell phenotypes as revealed by proliferation potential, differentiation ability, gene expression (i.e., Oct-4), a high frequency of anchorage-independent growth, and the presence of serpinginous-shaped cells in a population of mostly fibroblast-like cells (FIG. 27(a)).

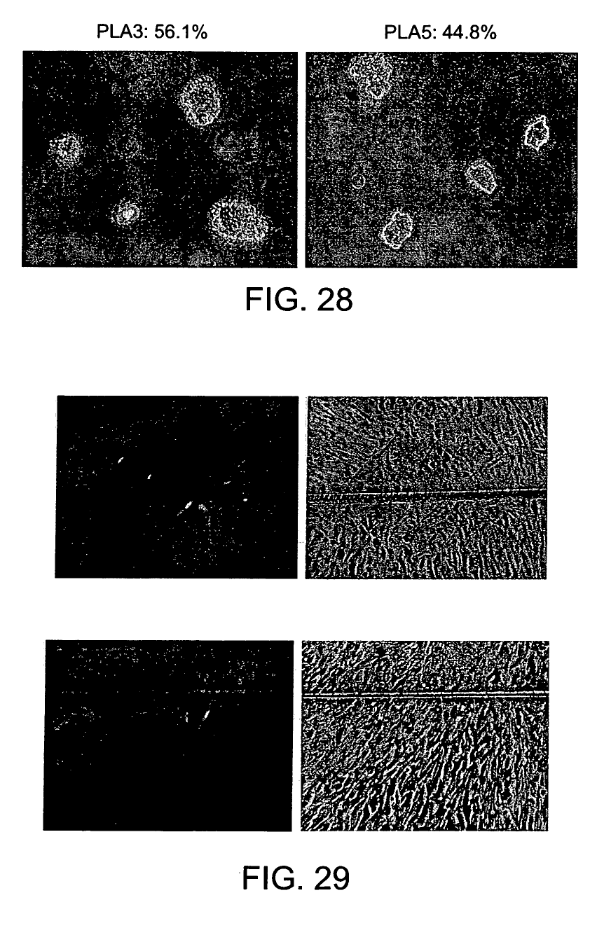

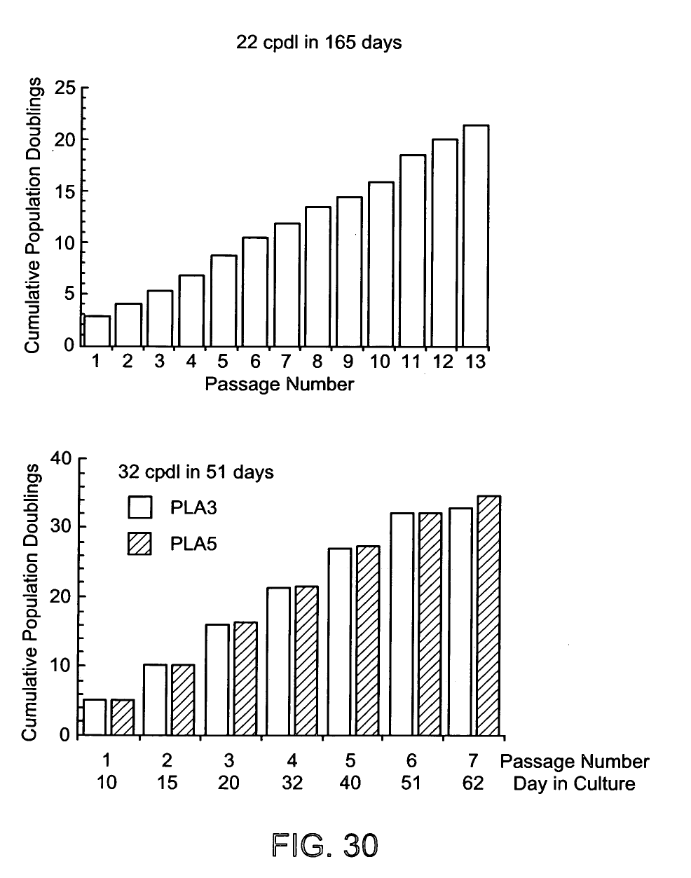

Individual cultures of these adipose-derived cells (mesenchymal stem cells) from different subjects that were grown in the medium described herein displayed anchorage- independent growth (i.e., colony-forming efficiency on plastic) of from 44.8%-56.1% (FIG. 28). This is a significantly higher percentage of anchorage-independent growth (about 50% AIG) than is observed in fibroblasts, which displayed 0.24% AIG. These data demonstrate that mesenchymal stem/precursor cells developed by the methods described herein had high frequency of anchorage independent growth (45% to 56%) The adipocyte stem cells cultured in the K-NAC medium described herein containing NAC and Asc-2P also displayed higher cumulative population doubling levels in a shorter time than has been previously reported for adipose tissue-derived cells (FIG. 30). The cells cultured in the K-NAC medium yielded a larger number of cells in shorter period of time compared to a previous report (Zuk et al. (2001) Tissue Eng. 7:211-228) (32 cpdl in 51 days compared to 22 cpdl in 165 day) and a colony-forming efficiency on a plastic surface of about 22%-38.2%.

Adipocyte stem cells were also examined for gap junctional intercellular communication. In these experiments, cultures containing serpiginous cells and precursor cells were assayed for GJIC assayed using the scrape loading/Lucifer yellow dye transfer method. These experiments demonstrated that the serpiginous shaped cells generally lacked gap junctional intercellular communication as shown by dye retention (FIG. 29), while the precursor cells had GJIC, as shown by their ability to transfer dye. In addition, the serpiginous cells could divide symmetrically (e.g., one cell into two serpiginous cells) or asymmetrically (one cell into one serpiginous and one cuboidal or fibroblast-like cell) (FIG. 27(b)).

These data demonstrate that the methods described herein using relatively low calcium concentrations (e.g., 0.09 mM), supplemented with NAC and ascorbate or other anti-oxidant compounds are useful for culturing and propagating stem cells derived from multiple tissue types. Furthermore, the stem cells retain their ability to differentiate into specific cell types upon induction with differentiation agents.

6.18 Differentiation Potential of Adipocyte-Derived Stem Cells

Adipocyte stem cells isolated and cultured under the conditions described herein (i.e., in K-NAC medium containing NAC and Asc-2P) were tested for their ability to differentiate into multiple cell types using methods known in the art (see Table 1).

Osteogenic differentiation

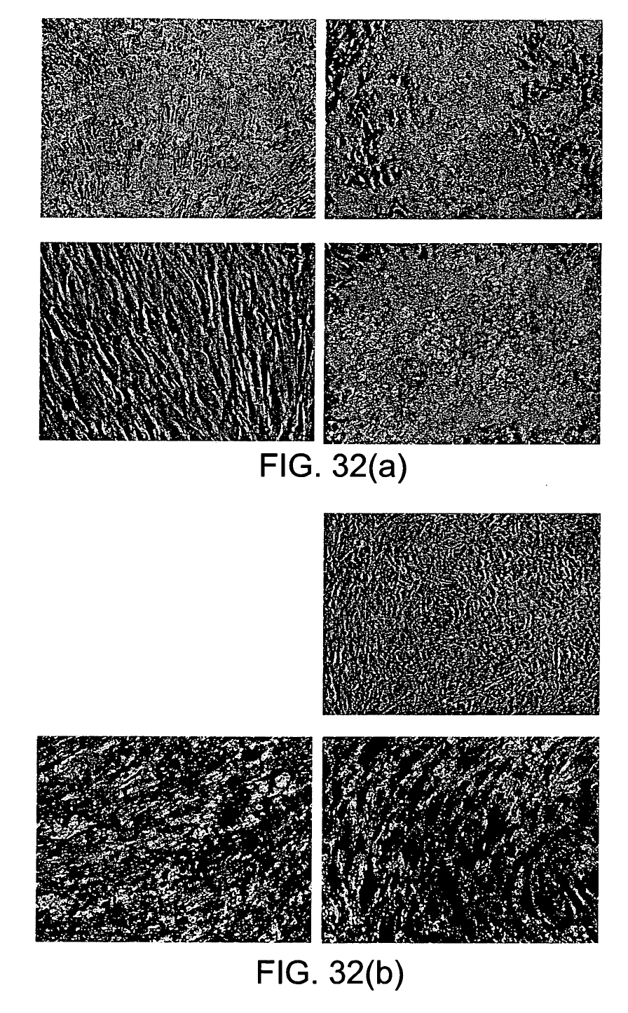

Adipose stem cells were tested for their ability to differentiate into osteocytes by culturing the stem cells in a medium composed of a modified MEM (Chang et al. (1981) Somatic Cell Genetics 7: 235) with 10% FBS and supplemented with 0.1 μM dexamethasone, 50 μM L-ascorbate-2-phosphate, and 10 mM 1-glycerophosphate disodium for about two to four weeks. Osteogenesis was identified by the presence of calcified extracellular matrix (ECM) in unstained cells (FIG. 32(a)) or using Von Kossa staining (FIG. 32(b)), the quantitative measurement of calcium in calcified ECM (Zuk et al. (2001) Tissue Engineering 7: 211), and the quantitative measurement of calcium in culture medium.

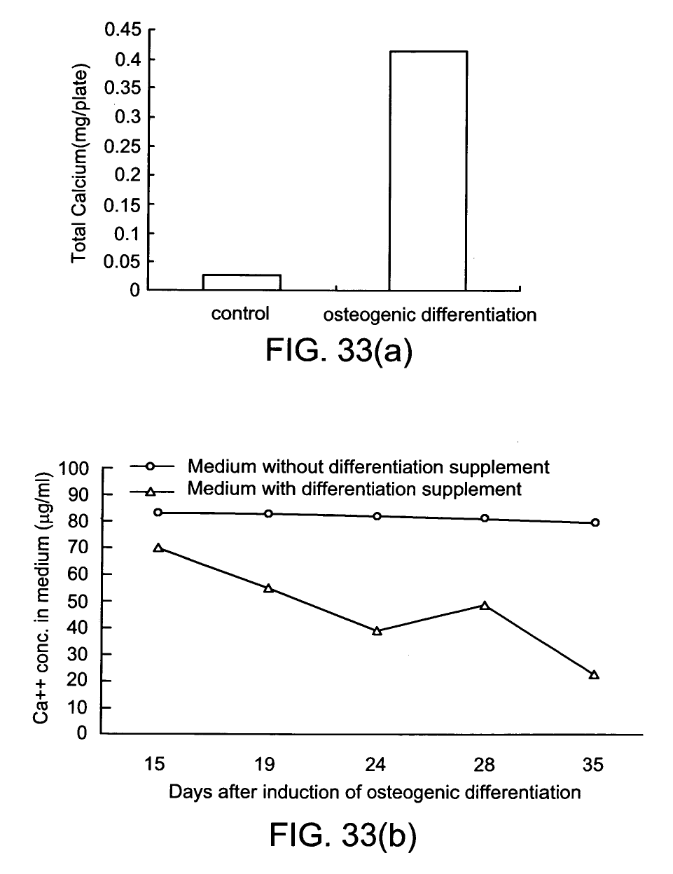

Von Kossa staining was used to identify calcium deposits, a feature of osteogenesis. After a four week incubation in differentiation medium as described above, the ECM of the cultured cells exhibited significant amounts of staining for calcium (FIG. 32(b)). In addition, the amount of calcium in the culture medium decreased in medium containing differentiation supplement compared to a control that lacked the supplement (FIG. 33(b)), suggesting that calcium is removed from medium as it is fixed by differentiating osteocytes. Calcified ECM is also a feature of osteogenic differentiation. The amount of calcium in calcified ECM was found to be at least about sixteen times in cells with differentiation induction greater than in control cells without treatment (FIG. 33 (a)).

These data demonstrate the putative mesenchymal stem cells cultured in a medium described herein have the ability to differentiate into osteocytes. Osteocytes are useful, e.g., for therapies related to bone repair.

Adipocyte differentiation

To further test the ability of mesenchymal stem cells obtained and cultured as described herein to differentiate into multiple cell types, the ability of these cells to differentiate into adipocytes was tested. Adipocyte induction was performed by culturing adipocyte stem cells in a modified MEM with 10% FBS supplemented with 500 μM IBMX (3-Isobutyl-1-methylxanthine), 11M dexamethasone, 1 μM indomethacin, and 10 μg/ml insulin for two days followed by one day in modified MEM medium supplemented with 10 μg/ml insulin and repeating the cycle two more times. Induction of adipocytes was determined using Oil Red 0 staining.

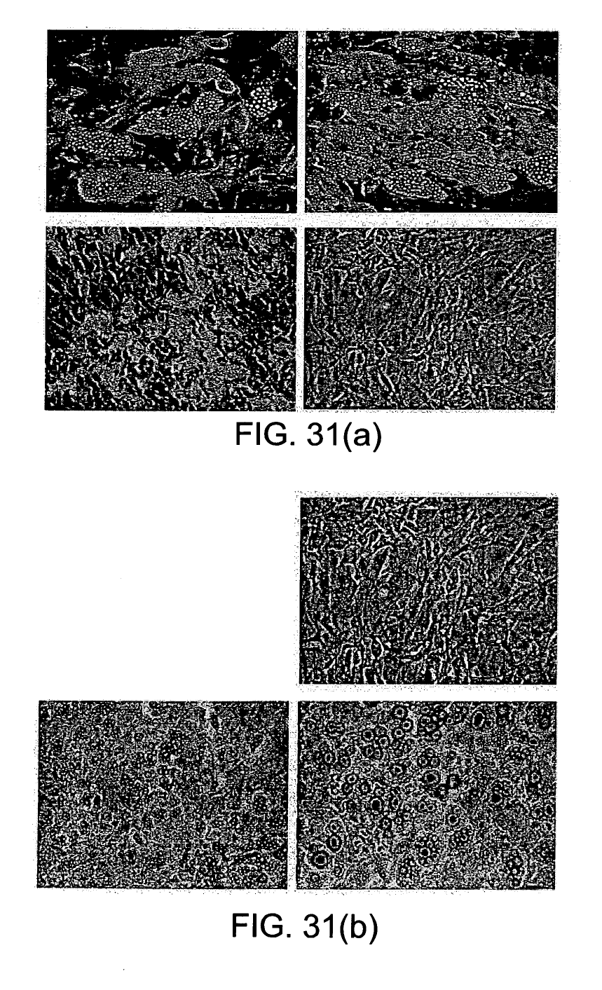

Oil Red 0 staining was readily detected in the induced cells, indicating that the putative mesenchymal stem cells cultured in a medium described herein can differentiate into adipocytes (FIGS. 31(b)). Some difference in vacuoles was seen observed in unstained cells (FIG. 31(a)). Adipocytes are useful, e.g., for cosmetic and reconstructive surgery.

Chondrogenic Differentiation

The ability of adipocyte stem cells obtained and cultured as described herein to differentiate into chondrocytes was tested. In these experiments, mesenchymal stem cells were subjected to micromass culture (1×10⁵ cells per cell aggregate in each well of a 24-well plate) using a modified MEM containing 10% FBS and supplemented with 10 ng/ml TGF-β1, 50 μM L-ascorbate-2-phosphate, and 6.25 μg/ml insulin. The induction medium was renewed once every 3 days.

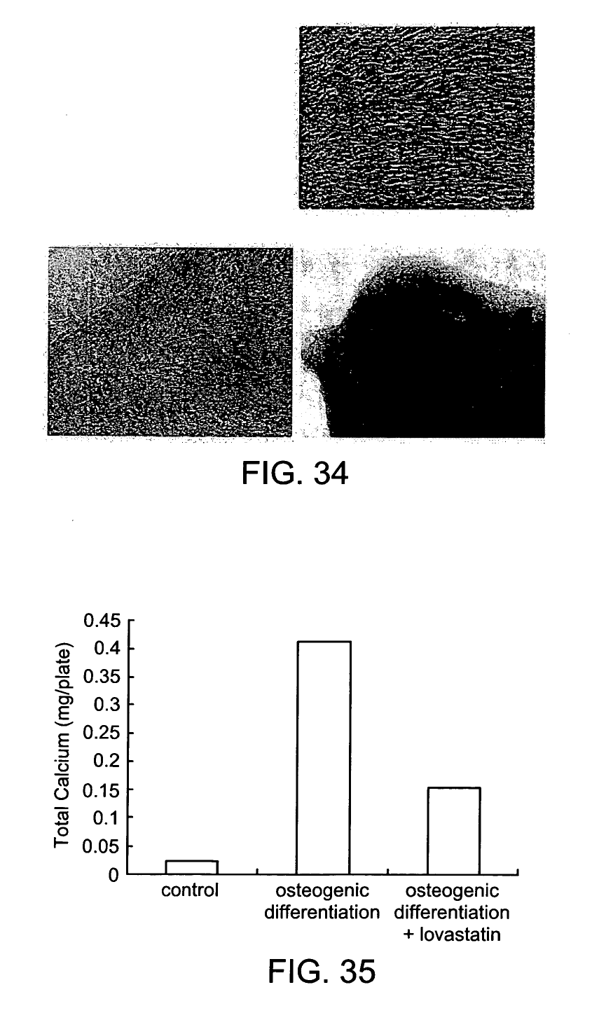

It was found that cells with characteristics of chondrocytes generally develop in about two weeks and could be identified using 1% Alcian blue 8GX in 0.1N HCl (pH 1.0) staining, which detects the presence of sulfated proteoglycans (FIG. 34). These data further show that the putative mesenchymal stem cells obtained and cultured in a medium described herein can differentiate into chondrocytes, which are useful, e.g., for therapies related to cartilage repair.

Myogenic Differentiation

To further demonstrate the differentiative potential of adipocyte stem cells cultured using a medium described herein, adipocyte stem cells were cultured in medium suitable for inducing myogenic differentiation. The differentiation medium was a modified MEM containing 5% horse serum and supplemented with 50 μM hydrocortisone and the cells were cultured for four to six weeks.

Differentiated cells were identified by their morphology and by immunostaining with an antibody that specifically recognizes skeletal myosin using methods known in the art. The cultures contained myogenic cells by 4-6 weeks after the initiation of incubation in the differentiation medium. Thus, the putative mesenchymal stem cells described herein have the potential to differentiate into myoblasts.

Taken together, these data demonstrate that mesenchymal stem cells (e.g., adipose stem cells) obtained and cultured using medium described herein (e.g., a medium containing relatively low concentrations of calcium, NAC, and L-ascorbate-2-phosphate), have the potential to differentiate into multiple cell types. Furthermore, the data provided herein are exemplary and are not intended to illustrate the complete range of cell types into which mesenchymal stem cells from adipose tissues can be cultured and differentiated.

The methods of inducing differentiation that are described herein are exemplary and are not intended to be limiting. Other suitable methods of identifying specific differentiated cell types are known in the art and can be used to identify differentiated cells induced to form from adult stem cells cultured using the methods described herein.

6.19 Statins and Osteoporosis

Cell culture models for various disorders are useful, e.g., for testing the ability of a compound to modulate a cellular process associated with the disorder. The adipocyte stem cells described herein are useful, e.g., for providing a pool of cells that can be differentiated at will and used in assays of such compounds.

Osteoporosis is a disorder that occurs when the amount of bone removed from the skeleton by bone-resorbing osteoclasts exceeds the amount of bone formed by osteoblasts. Statins may decrease bone resorption by preventing osteoclast activation that is mediated by farnesyl-pp and geranylgeranyl-pp (Cruz et al., (2002) Cleveland Clinic J. Med. 69:277-288). Experiments were performed to test the effect of lovastatin on the formation of calcified ECM using adipocyte cells that were differentiated into osteocytes as described supra. Briefly, control cells (adipose tissue-derived mesenchymal stem cells) were compared to mesenchymal stem cells (adipose stem cells) that were grown in osteogenic medium with 0.2 μM lovastatin (experimental), or in osteogenic medium without lovastatin (induced control). In these experiments, lovastatin was added to the experimental cultures at the same time as the osteogenic medium. Microscopic inspection of the cultures revealed significantly less formation of calcified ECM in the experimental cultures compared to the induced control cultures. Measurements of total calcium (mg/plate) confirmed this observation (FIG. 35). These data demonstrate that lovastatin can affect osteoblast differentiation and thus, the use of statins for treating osteoporosis should be subjected to further examination.

In general, these experiments demonstrate that mesenchymal stem cells (e.g., derived from adipose tissue) obtained and cultured using medium and methods described herein) can be used to test compounds for their ability to affect disease-related cellular activities and are therefore useful for testing the suitability of such compounds as candidate drug compounds.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A method of obtaining isolated adult stem cells, the method comprising:
a) providing a population of dissociated cells comprising stem cells from an adult tissue;
b) culturing the population of dissociated cells in a cell culture medium comprising a low calcium concentration and an effective amount of one or more of N-acetyl-L-cysteine, an antioxidant, and nicotinamide; and
c) allowing adult stem cell colonies to develop in the cell culture medium, thereby yielding a population of adult stem cells.

2. The method of claim 1, wherein the medium is a modified MCDB 153 medium.

3. The method of claim 1, wherein the isolated stem cells are from a primate.

4. The method of claim 3, wherein the primate is a human.

5. The method of claim 1, wherein the isolated adult stem cell population is clonal in origin.

6. The method of claim 1, wherein the isolated adult stem cell population is multi-clonal in origin.

7. The method of claim 1, wherein the isolated adult stem cells are cultured on tissue culture plastic and can form colonies.

8. The method of claim 1, wherein the population of isolated adult cells is obtained without the use of feeder cells.

9. The method of claim 1, wherein the population of isolated adult cells has a high proliferation potential.

10. The method of claim 9, wherein the stem cells are mesenchymal stem cells, and the proliferation potential is at least 48 cell divisions.

11. The method of claim 9, wherein the stem cells are liver stem cells and the proliferation potential is about 32 cell divisions.

12. The method of claim 1, further comprising immortalizing an adult stem cell by transforming the isolated adult stem cell with an immortalizing gene.

13. The method of claim 12, wherein the immortalizing gene encodes SV40 large T- antigen.

14. The method of claim 12, wherein the immortalizing gene is selected from the group consisting of a gene encoding dominant-negative p53, dominant-negative RB, hTERT, adenovirus E1a, adenovirus E1b, papilloma virus E6, and papilloma virus E7.

15. The method of claim 1, wherein the low calcium concentration is less than about 0.3 mM.

16. The method of claim 1, wherein the low calcium concentration is less than about 0.2 mM.

17. The method of claim 1, wherein the low calcium concentration is less than about 0.1 mM.

18. The method of claim 1, wherein the low calcium concentration is about 0.04 mM to about 0.18 mM.

19. The method of claim 1, wherein the low calcium concentration is about 0.06 mM to about 0.12 mM.

20. The method of claim 1, wherein the low calcium concentration is about 0.08 mM to about 0.10 mM.

21. The method of claim 1, wherein the low calcium concentration is about 0.09 mM.

22. The method of claim 1, wherein the antioxidant is vitamin C.

23. The method of claim 22, wherein the vitamin C is L-ascorbic acid-2-phosphate.

24. The method of claim 23, wherein the L-ascorbic acid-2-phosphate is provided at a concentration of at least about 0.05 mM.

25. The method of claim 23, wherein the L-ascorbic acid-2-phosphate is provided at about 0.2 mM.

26. The method of claim 1, wherein the antioxidant is selected from the group consisting of vitamin C, vitamin E, N-acetyl-L-cysteine, resveratrol, coenzyme Q, alpha-lipoic acid, lycopene, bioflavonoids, and quercetin.

27. The method of claim 1, wherein the N-acetyl-L-cysteine concentration is at least about 0.5 mM.

28. The method of claim 27, wherein the N-acetyl-L-cysteine concentration is about 2 mM.

29. The method of claim 1, wherein the nicotinamide concentration is at least about 1 mM.

30. The method of claim 29, wherein the nicotinamide concentration is about 5 mM to 10 mM.

31. The method of claim 1, wherein the cell culture medium further comprises a growth factor and hormone selected from the group consisting of EGF (epidermal growth factor), insulin, hydrocortisone, and 3,3′,5-triiodo-D,L-thyronine.

32. The method of claim 1, wherein the cell culture medium further comprises bovine pituitary extract.

33. The method of claim 1, wherein the cell culture medium further comprises fetal bovine serum.

34. The method of claim 33, wherein the cell culture medium further comprises bovine pituitary extract.

35. The method of claim 1, wherein the cell culture medium further comprises at least one of 5 ng/ml of recombinant human EGF, 5 μg/ml of insulin, 74 ng/ml of hydrocortisone, 10 nM 3,3′,5-triiodo-D.L-thyronine, bovine pituitary extract, and 5% to 10% fetal bovine serum.

36. The method of claim 1 or 12, further comprising culturing an isolated adult stem cell under conditions such that the cell expresses one or more tissue-specific functions.

37. The method of claim 36, wherein the isolated adult stem cell is derived from adult adipose tissue and can differentiate into a chondrocyte, myoblast, osteoblast, neuronal cell, or adipocyte.

38. The method of claim 36, wherein the tissue-specific function is selected from the group consisting of positive Oil Red 0 staining for lipid vacuoles, Von Kossa staining for calcification of ECM, immunostaining for skeletal myosin expression, and Alcian Blue staining for sulfated proteoglcan accumulation by chondrocytes.

39. The method of claim 36, wherein the adult stem cells are differentiated by contact with a medium comprising at least about 0.6 mM calcium.

40. The method of claim 36, wherein the adult stem cell is derived from adipose tissue and is differentiated by contact with a chrondrocyte differentiation agent, myoblast differentiation agent, osteoblast differentiation agent, or adipocyte differentiation agent.

41. The method of claim 40, wherein the differentiation agent comprises TGF-β1, L- ascorbate-2-phosphate, and insulin, and the cell differentiates into a chondrocyte.

42. The method of claim 40, wherein the differentiation agent comprises hydrocortisone, and the cell differentiates into a myoblast.

43. The method of claim 40, wherein the differentiation agent comprises IBMX, dexamethasone, indomethasone, and insulin, and the cell differentiates into an adipocyte.

44. The method of claim 40, further comprising:
i. incubating the cell in a differentiation agent comprising IBMX, dexamethasone, indomethasone, and insulin for two days;
ii. incubating the cell in insulin for one day; and
iii. repeating steps i and ii two additional times,
wherein the cell differentiates into an adipocyte.

45. The method of claim 40, wherein the differentiation agent comprises dexamethasone, L-ascorbate-2-phosphate, and β-glycerophosphate, and the cell differentiates into an osteocyte.

46. The method of claim 36, further comprising providing the differentiated adult stem cell expressing one or more tissue-specific functions to a subject in need thereof.

47. The method of claim 1, further compromising providing an isolated adult stem cell to a subject in need thereof.

48. The method of claim 12, further compromising providing an isolated adult stem cell to a subject in need thereof.

49. The method of claim 47, wherein the subject is a human having a disease, disorder, or other dysfunction of adipose tissue, bone, cartilage, or muscle.

50. The method of claim 49, wherein the disease, disorder or other dysfunction of the adipose tissue, bone, cartilage, or muscle is selected from the group consisting of osteoporosis, bone damage, osteoarthritis, muscular dystrophy, myocardial infarction, reconstructive surgery, and spinal cord injury.

51. The method of claim 1, wherein the population of dissociated adult cells are cultured in a medium comprising an effective amount of at least two of N-acetyl-L-cysteine, nicotinamide, and an antioxidant.

52. The method of claim 51, wherein the population of dissociated adult cells are cultured in a medium comprising an effective amount of N-acetyl-L-cysteine, nicotinamide, and an antioxidant.

53. The method of claim 52, wherein the population of dissociated adult cells are cultured in a medium comprising about 2 mM N-acetyl-L-cysteine, about 5 mM to 10 mM nicotinamide, and about 0.2 mM L-ascorbic acid-2-phosphate.

54. The method of claim 1, wherein the adult tissue is adipose tissue and the stem cell is mesenchymal stem cell.

55. The method of claim 12, wherein the adult tissue is adipose tissue and the stem cell is mesenchymal stem cell.

56. The method of claim 1, wherein the adult tissue is liver tissue and the stem cell is a liver stem cell.

57. The method of claim 11, wherein the adult tissue is liver tissue and the stem cell is a liver stem cell.

58. A cell culture medium comprising a low calcium ion concentration and an effective amount of one or more of N-acetyl-L-cysteine, nicotinamide, and an antioxidant.

59. The cell culture medium of claim 58, comprising an effective amount of at least two of N-acetyl-L-cysteine, nicotinamide, and an antioxidant.

60. The cell culture medium of claim 59, comprising an effective amount of N-acetyl-L- cysteine, nicotinamide, and an antioxidant.

61. The cell culture medium of claim 60, comprising about 2 mM N-acetyl-L-cysteine, about 5 mM to 10 mM nicotinamide, and about 0.2 mM L-ascorbic acid-2-phosphate.

62. The cell culture medium of claim 58, wherein the low calcium ion concentration is less than about 0.2 mM.

63. The cell culture medium of claim 58, wherein the low calcium ion concentration is about 0.04 mM to about 0.18 mM.

64. The cell culture medium of claim 58, wherein the low calcium ion concentration is about 0.08 mM to about 0.10 mM.

65. The cell culture medium of claim 58, wherein the low calcium ion concentration is about 0.09 mM.

66. A cell culture medium comprising:
(a) a low calcium ion concentration;
(b) and an effective amount of one or more of an agent that promotes intracellular glutathione synthesis;
(c) an inhibitor of poly ADP-ribose polymerase; and
(d) an antioxidant.

67. The cell culture medium of claim 66, comprising:
(a) an effective amount of at least one agent that promotes intracellular glutathione synthesis;
(b) an inhibitor of poly ADP-ribose polymerase; and
(c) an antioxidant.

68. The cell culture medium of claim 67, comprising:
(a) an effective amount of an agent that promotes intracellular glutathione synthesis;
(b) an inhibitor of poly ADP-ribose polymerase; and
(c) an antioxidant.

69. The cell culture medium of claim 68, comprising about 2 mM N-acetyl-L-cysteine, about 5 to 10 mM nicotinamide, and about 0.2 mM L-ascorbic acid-2-phosphate.

70. The cell culture medium of claim 66, wherein the low calcium ion concentration is less than about 0.2 mM.

71. The cell culture medium of claim 66, wherein the low calcium ion concentration is about 0.04 mM to about 0.18 mM.

72. The cell culture medium of claim 66, wherein the low calcium ion concentration is about 0.08 mM to about 0.10 mM.

73. The cell culture medium of claim 66, wherein the low calcium ion concentration is about 0.09 mM.

74. A cell culture medium for adult human stem cells, said medium comprising
(a) a calcium ion concentration of 0 to about 0.5 mM;
(b) at least about 1 mM N-acetyl-L-cysteine;
(c) at least about 1 mM nicotinamide; and
(d) an effective amount of an antioxidant agent, wherein the cell culture medium is sufficient for culturing adult human stem cells.

75. The cell culture medium of claim 74, wherein the calcium concentration is 0 to about 0.2 mM.

76. The cell culture medium of claim 74, wherein the calcium concentration is about 0.04-0.18 mM.

77. The cell culture medium of claim 74, wherein the calcium ion concentration is about 0.05 mM to about 0.1 mM.

78. The cell culture medium of claim 74, wherein the antioxidant is vitamin C.

79. The cell culture medium of claim 78, wherein the vitamin C is L-ascorbic acid-2-phosphate.

80. The cell culture medium of claim 79, wherein the L-ascorbic acid-2-phosphate is provided at a concentration of at least about 0.1 mM.

81. The cell culture medium of claim 80, wherein the L-ascorbic acid-2-phosphate is provided at a concentration of at about 0.2 mM.

82. The cell culture medium of claim 74, wherein the antioxidant is selected from the group consisting of vitamin C, vitamin E, N-acetyl-L-cysteine, and resveratrol.

83. The cell culture medium of claim 74, wherein the N-acetyl-L-cysteine concentration is at least about 1 mM.

84. The cell culture medium of claim 74, wherein nicotinamide concentration is at least about 2 mM.

85. The cell culture medium of claim 74, wherein the cell culture medium further comprises at least one of EGF, insulin, hydrocortisone, 3,3′,5-triiodo-D.L-thyronine, bovine pituitary extract, or fetal bovine serum.

86. The cell culture medium of claim 74, wherein the cell culture medium further comprises at least one of 5 ng/ml of recombinant human EGF, 5 μg/ml of insulin, 74 ng/ml of hydrocortisone, 10 nM 3,3′,5-triiodo-D.L-thyronine, 50 μg/1 ml bovine pituitary extract, and 10% fetal bovine serum.

87. The cell culture medium of claim 74, wherein the cell culture medium is used for culturing adult stem cells derived from adipose tissue.

88. The cell culture medium of claim 74, wherein the cell culture medium is used for culturing adult stem cells derived from liver tissue.

89. An isolated adult human mesenchymal stem cell which:
(a) expresses Oct-4 and/or vimentin;
(b) does not possess a gap-junction intercellular communication activity; and
(c) has a high proliferation potential of at least about 20 cell divisions.

90. The isolated adult human mesenchymal stem cell of claim 86, wherein the cell, or a progeny cell derived from the cell, can differentiate into an adipocyte, osteocyte, chondrocyte, neuronal cell, or skeletal muscle cell.

91. An isolated adult human liver stem cell which:
(a) expresses Oct-4, alpha-fetoprotein, Thy-1, and/or vimentin;
(b) does not possess a gap-junction intercellular communication activity; and
(c) has a high proliferation potential of at least about 20 cell divisions.

92. The isolated adult human liver stem cell of claim 91, wherein the cell, or a progeny cell derived from the cell, can differentiate into a hepatocyte.
